(19) United States
(12) Patent Application Publication
(10) Pub. No.: US 2025/0029677 A1
(43) Pub. Date: Jan. 23, 2025

(54) TECHNIQUES FOR IDENTIFYING HER2-LOW BREAST CANCER TUMORS

(71) Applicant: BostonGene Corporation, Waltham, MA (US)

(72) Inventors: Polina Turova, Yerevan (AM); Vladimir Kushnarev, Yerevan (AM); Oleg Baranov, Yerevan (AM); Anna Butusova, Yerevan (AM); Sofiia Menshikova, Yerevan (AM); Konstantin Chernyshov, Yerevan (AM); Nikita Kotlov, Waltham, MA (US)

(73) Assignee: BostonGene Corporation, Waltham, MA (US)

(21) Appl. No.: 18/767,248

(22) Filed: Jul. 9, 2024

Related U.S. Application Data
(60) Provisional application No. 63/605,215, filed on Dec. 1, 2023.
(60) Provisional application No. 63/512,865, filed on Jul. 10, 2023.

(51) Int. Cl. G16B 25/10; G16B 40/20; G16H 20/17; G16H 50/20

(52) U.S. Cl. CPC G16B 25/10; G16B 40/20; G16H 20/17; G16H 50/20

(57) ABSTRACT

Aspects of the disclosure relate to methods, systems, and computer-readable storage media, which are useful for characterizing subjects having certain cancers, for example breast cancer. The disclosure is based, in part, on methods for determining the molecular breast cancer (BC) type of a subject and identifying the subject's prognosis and/or one or more therapeutic agents for treating the subject based upon the molecular BC type determination.

### CROSS-REFERENCE TO RELATED APPLICATIONS

This application claims the benefit under 35 U.S.C. § 119(e) of the filing date of U.S. Provisional Application No. 63/605,215, filed Dec. 1, 2023, and entitled “TECHNIQUES FOR IDENTIFYING HER2-LOW BREAST CANCER TUMORS”, and U.S. Provisional Application No. 63/512,865, filed Jul. 10, 2023, and entitled “TECHNIQUES FOR IDENTIFYING HER2-LOW BREAST CANCER”, the entire contents of each of which are incorporated by reference herein.

### BACKGROUND

Breast cancers (BCs) are segregable into subtypes based on their intrinsic molecular features. When evaluating HER2 status, clinicians often rely on immunohistochemistry (IHC) analysis, which is plagued by reproducibility issues. The widely used PAM50 transcriptomic system also does not include account for low HER2-expressing samples. Addressing these limitations now is particularly important given the recent approval of a targeted therapy for HER2-low BCs.

### SUMMARY

Some aspects provide for a method for identifying HER2-low breast cancer from RNA expression data of a tumor sample from a subject having breast cancer using a plurality of trained machine learning classifiers including first, second, and third trained machine learning classifiers associated with respective first, second, and third sets of genes, the method comprising: using at least one computer hardware processor to perform: obtaining the RNA expression data, the RNA expression data specifying RNA expression levels at least for genes in the first, second, and third sets of genes, the RNA expression data having been previously obtained from the tumor sample; determining, using the RNA expression levels for the first set of genes and the first trained machine learning classifier, whether the tumor sample has a Basal molecular subtype; when it is determined that the tumor sample does not have the Basal molecular subtype, determining, using the RNA expression levels for the second set of genes and the second trained machine learning classifier, whether the tumor sample has a HER2-high molecular subtype; and when it is determined that the tumor sample does not have the HER2-high molecular subtype, determining, using the RNA expression levels for the third set of genes and the third trained machine learning classifier, that the tumor sample has a HER2-low molecular subtype.

Some aspects provide for a system, comprising: at least one computer hardware processor; and at least one non-transitory computer-readable storage medium storing processor-executable instructions that, when executed by the at least one computer hardware processor, cause the at least one computer hardware processor to perform a method for identifying HER2-low breast cancer from RNA expression data of a tumor sample from a subject having breast cancer using a plurality of trained machine learning classifiers including first, second, and third trained machine learning classifiers associated with respective first, second, and third sets of genes, the method comprising: using at least one computer hardware processor to perform: obtaining the RNA expression data, the RNA expression data specifying RNA expression levels at least for genes in the first, second, and third sets of genes, the RNA expression data having been previously obtained from the tumor sample; determining, using the RNA expression levels for the first set of genes and the first trained machine learning classifier, whether the tumor sample has a Basal molecular subtype; when it is determined that the tumor sample does not have the Basal molecular subtype, determining, using the RNA expression levels for the second set of genes and the second trained machine learning classifier, whether the tumor sample has a HER2-high molecular subtype; and when it is determined that the tumor sample does not have the HER2-high molecular subtype, determining, using the RNA expression levels for the third set of genes and the third trained machine learning classifier, that the tumor sample has a HER2-low molecular subtype.

Some aspects provide for at least one non-transitory computer-readable storage medium storing processor-executable instructions that, when executed by at least one computer hardware processor, cause the at least one computer hardware processor to perform a method for identifying HER2-low breast cancer from RNA expression data of a tumor sample from a subject having breast cancer using a plurality of trained machine learning classifiers including first, second, and third trained machine learning classifiers associated with respective first, second, and third sets of genes, the method comprising: using at least one computer hardware processor to perform: obtaining the RNA expression data, the RNA expression data specifying RNA expression levels at least for genes in the first, second, and third sets of genes, the RNA expression data having been previously obtained from the tumor sample; determining, using the RNA expression levels for the first set of genes and the first trained machine learning classifier, whether the tumor sample has a Basal molecular subtype; when it is determined that the tumor sample does not have the Basal molecular subtype, determining, using the RNA expression levels for the second set of genes and the second trained machine learning classifier, whether the tumor sample has a HER2-high molecular subtype; and when it is determined that the tumor sample does not have the HER2-high molecular subtype, determining, using the RNA expression levels for the third set of genes and the third trained machine learning classifier, that the tumor sample has a HER2-low molecular subtype.

Some aspects provide for a method for identifying a molecular subtype of breast cancer for a subject, the method comprising: using at least one computer hardware processor to perform: obtaining RNA expression data, the RNA expression data having been previously obtained from a tumor sample from a subject having breast cancer; and identifying, from among multiple breast cancer molecular subtypes and using the RNA expression data and a plurality trained machine learning classifiers, a molecular subtype for the tumor sample, the multiple breast cancer molecular subtypes comprising: a Basal subtype, a HER2-high subtype, a HER2-low subtype, Luminal subtype A, and Luminal subtype B.

Some aspects provide for a system, comprising: at least one computer hardware processor; and at least one non-transitory computer-readable storage medium storing processor-executable instructions that, when executed by the at least one computer hardware processor, cause the at least one computer hardware processor to perform a method for identifying a molecular subtype of breast cancer for a subject, the method comprising: using at least one computer hardware processor to perform: obtaining RNA expression data, the RNA expression data having been previously obtained from a tumor sample from a subject having breast cancer; and identifying, from among multiple breast cancer molecular subtypes and using the RNA expression data and a plurality trained machine learning classifiers, a molecular subtype for the tumor sample, the multiple breast cancer molecular subtypes comprising: a Basal subtype, a HER2-high subtype, a HER2-low subtype, Luminal subtype A, and Luminal subtype B.

Some aspects provide for at least one non-transitory computer-readable storage medium storing processor-executable instructions that, when executed by at least one computer hardware processor, cause the at least one computer hardware processor to perform a method for identifying a molecular subtype of breast cancer for a subject, the method comprising: using at least one computer hardware processor to perform: obtaining RNA expression data, the RNA expression data having been previously obtained from a tumor sample from a subject having breast cancer; and identifying, from among multiple breast cancer molecular subtypes and using the RNA expression data and a plurality trained machine learning classifiers, a molecular subtype for the tumor sample, the multiple breast cancer molecular subtypes comprising: a Basal subtype, a HER2-high subtype, a HER2-low subtype, Luminal subtype A, and Luminal subtype B.

Embodiments of any of the above aspects may have one or more of the following features.

Some embodiments further comprise: when it is determined that the tumor sample has the HER2-low molecular subtype, recommending that the subject be treated with trastuzumab deruxtecan.

Some embodiments further comprise: administering trastuzumab deruxtecan to the subject.

In some embodiments, the first set of genes includes at least some genes (e.g., 2, 3, 4, 5, or more genes) selected from the group consisting of FOXA1, MLPH, FOXC1, SFRP1, NAT1, ORC6, BIRC5, CDC20, AGR2, AR, CA12, and CDK1. In some embodiments, determining whether the tumor sample has a Basal molecular subtype comprises: generating a first input using expression levels for genes in the first set of genes, and processing the first input using the first trained machine learning classifier to obtain a first output indicative of whether the tumor sample has the Basal molecular subtype.

In some embodiments, the first set of genes consists of the following genes: FOXA1, MLPH, FOXC1, SFRP1, NAT1, ORC6, BIRC5, CDC20, AGR2, AR, CA12, and CDK1.

In some embodiments, the first trained machine learning classifier is a gradient boosted decision tree classifier.

In some embodiments, generating the first input using expression levels for genes in the first set of genes comprises: determining ranks for the genes in the first set of genes based on expression levels for the genes in the first set of genes and expression levels of other genes in the RNA expression data; and creating the first input as a vector of the determined ranks for the genes in the first set of genes.

In some embodiments, the second set of genes includes at least some genes (e.g., 2, 3, 4, 5, or more genes) selected from the group consisting of MLPH, ESR1, FOXC1, MYC, PHGDH, ACTR3B, CDH3, KRT14, KRT5, EGFR, CDC6, FGFR4, MAPT, CXXC5, NAT1, MDM2, CCNE1, MYBL2, RRM2, NUF2, TYMS, ANLN, UBE2T, CENPF, PTTG1, UBE2C, CDC20, MELK, GRB7, ERBB2, FABP7, GATA3, AR, CCNB2, CDK1, CLDN8, ERBB3, IGF1R, PIK3CA, and TOP2A. In some embodiments, determining whether the tumor sample has a HER2-high molecular subtype comprises: generating a second input using expression levels for genes in the second set of genes, and processing the second input using the second trained machine learning classifier to obtain a second output indicative of whether the tumor sample has the HER2-high molecular subtype.

In some embodiments, the second set of genes consists of the following genes: MLPH, ESR1, FOXC1, MYC, PHGDH, ACTR3B, CDH3, KRT14, KRT5, EGFR, CDC6, FGFR4, MAPT, CXXC5, NAT1, MDM2, CCNE1, MYBL2, RRM2, NUF2, TYMS, ANLN, UBE2T, CENPF, PTTG1, UBE2C, CDC20, MELK, GRB7, ERBB2, FABP7, GATA3, AR, CCNB2, CDK1, CLDN8, ERBB3, IGF1R, PIK3CA, and TOP2A.

In some embodiments, the second trained machine learning classifier is a gradient boosted decision tree classifier.

In some embodiments, generating the second input using expression levels for genes in the second set of genes comprises: determining ranks for the genes in the second set of genes based on expression levels for the genes in the second set of genes and expression levels of other genes in the RNA expression data; and creating the second input as a vector of the determined ranks for the genes in the second set of genes.

In some embodiments, the third set of genes includes at least some genes (e.g., 2, 3, 4, 5, or more genes) selected from the group consisting of FOXA1, MLPH, ESR1, MYC, PHGDH, ACTR3B, SFRP1, KRT17, KRT5, EGFR, CDC6, FGFR4, MAPT, CXXC5, BCL2, PGR, NAT1, SLC39A6, BLVRA, CCNE1, MYBL2, RRM2, ORC6, NUF2, TYMS, ANLN, CENPF, EXO1, MKI67, BIRC5, UBE2C, KIF2C, CEP55, MELK, ERBB2, ACE2, FABP7, AKR1B15, GATA3, AGR3, AR, CA12, CDK1, CLDN8, E2F2, IGF1R, SOX11, TFF1, and TOP2A. In some embodiments, determining whether the tumor sample has a HER2-low molecular subtype comprises: generating a third input using expression levels for genes in the third set of genes, and processing the third input using the third trained machine learning classifier to obtain a third output indicating that the tumor sample has the HER2-low molecular subtype.

In some embodiments, the third set of genes consists of the following genes: FOXA1, MLPH, ESR1, MYC, PHGDH, ACTR3B, SFRP1, KRT17, KRT5, EGFR, CDC6, FGFR4, MAPT, CXXC5, BCL2, PGR, NAT1, SLC39A6, BLVRA, CCNE1, MYBL2, RRM2, ORC6, NUF2, TYMS, ANLN, CENPF, EXO1, MKI67, BIRC5, UBE2C, KIF2C, CEP55, MELK, ERBB2, ACE2, FABP7, AKR1B15, GATA3, AGR3, AR, CA12, CDK1, CLDN8, E2F2, IGF1R, SOX11, TFF1, and TOP2A.

In some embodiments, the third trained machine learning classifier is a gradient boosted decision tree classifier.

In some embodiments, generating the third input using expression levels for genes in the third set of genes comprises: determining ranks for the genes in the third set of genes based on expression levels for the genes in the third set of genes and expression levels of other genes in the RNA expression data; and creating the third input as a vector of the determined ranks for the genes in the third set of genes.

Some embodiments further comprise: generating the RNA expression data from sequencing data previously obtained by sequencing the tumor sample obtained from the subject.

In some embodiments, the sequencing data comprises at least 1 million reads, at least 5 million reads, at least 10 million reads, at least 20 million reads, at least 50 million reads, or at least 100 million reads.

In some embodiments, the sequencing data comprises whole exome sequencing (WES) data, bulk RNA sequencing (RNA-seq) data, single cell RNA sequencing (scRNA-seq) data, microarray data, and/or next generation sequencing (NGS) data.

Some embodiments further comprise: identifying a cancer therapy for the subject based on the identified molecular subtype for the tumor sample from the subject.

Some embodiments further comprise: administering the cancer therapy to the subject.

Some embodiments further comprise: when the identified molecular subtype for the tumor sample from the subject is HER2-low subtype, identifying trastuzumab deruxtecan as a cancer therapy for the subject.

Some embodiments further comprise: administering trastuzumab deruxtecan to the subject.

In some embodiments, the plurality of trained machine learning classifiers includes a first trained machine learning classifier associated with a first set of genes. In some embodiments, the RNA expression data specifies RNA expression levels for genes in the first set of genes. In some embodiments, identifying the molecular subtype for the tumor sample comprises: determining, using the RNA expression levels for the first set of genes and the first trained machine learning classifier, whether the tumor sample has the Basal molecular subtype.

In some embodiments, the first set of genes includes at least some (e.g., 2, 3, 4, 5, or more genes), optionally all, of the following genes: FOXA1, MLPH, FOXC1, SFRP1, NAT1, ORC6, BIRC5, CDC20, AGR2, AR, CA12, and CDK1.

In some embodiments, the first trained machine learning classifier is a gradient boosted decision tree classifier.

In some embodiments, the plurality of trained machine learning classifiers includes a second trained machine learning classifier associated with a second set of genes. In some embodiments, the RNA expression data specifies RNA expression levels for genes in the second set of genes. In some embodiments, identifying the molecular subtype for the tumor sample comprises: when it is determined that the tumor sample does not have the Basal molecular subtype, determining, using the RNA expression levels for the second set of set of genes and the second trained machine learning classifier, whether the tumor sample has the HER2-high molecular subtype.

In some embodiments, the second set of genes includes at least some (e.g., 2, 3, 4, 5, or more genes), optionally all, of the following genes: MLPH, ESR1, FOXC1, MYC, PHGDH, ACTR3B, CDH3, KRT14, KRT5, EGFR, CDC6, FGFR4, MAPT, CXXC5, NAT1, MDM2, CCNE1, MYBL2, RRM2, NUF2, TYMS, ANLN, UBE2T, CENPF, PTTG1, UBE2C, CDC20, MELK, GRB7, ERBB2, FABP7, GATA3, AR, CCNB2, CDK1, CLDN8, ERBB3, IGF1R, PIK3CA, and TOP2A.

In some embodiments, the second trained machine learning classifier is a gradient boosted decision tree classifier.

In some embodiments, the plurality of trained machine learning classifiers includes a third trained machine learning classifier associated with a third set of genes. In some embodiments, the RNA expression data specifies RNA expression levels for genes in the third set of genes. In some embodiments, identifying the molecular subtype for the tumor sample comprises: when it is determined that the tumor sample does not have the HER2-high molecular subtype, determining, using the RNA expression levels for the third set of set of genes and the third trained machine learning classifier, whether the tumor sample has the HER2-low molecular subtype.

In some embodiments, the third set of genes includes at least some (e.g., 2, 3, 4, 5, or more genes), optionally all, of the following genes: FOXA1, MLPH, ESR1, MYC, PHGDH, ACTR3B, SFRP1, KRT17, KRT5, EGFR, CDC6, FGFR4, MAPT, CXXC5, BCL2, PGR, NAT1, SLC39A6, BLVRA, CCNE1, MYBL2, RRM2, ORC6, NUF2, TYMS, ANLN, CENPF, EXO1, MKI67, BIRC5, UBE2C, KIF2C, CEP55, MELK, ERBB2, ACE2, FABP7, AKR1B15, GATA3, AGR3, AR, CA12, CDK1, CLDN8, E2F2, IGF1R, SOX11, TFF1, and TOP2A.

In some embodiments, the third trained machine learning classifier is a gradient boosted decision tree classifier.

In some embodiments, the RNA expression data specifies RNA expression levels for genes in fourth and fifth sets of genes. In some embodiments, identifying the molecular subtype for the tumor sample comprises: when it is determined that the tumor sample does not have the HER2-low molecular subtype, identifying that the tumor sample has a Luminal A or Luminal B subtype at least in part by: determining a first enrichment score for the fourth set of genes; determining a second enrichment score for the fifth set of genes; providing the first and second enrichment scores as inputs to a logistic regression model to obtain an output indicating whether the tumor sample is Ki67 positive or Ki67 negative; when it is determined, based on the output of the logistic regression model, that the tumor sample is Ki67 negative, determining that the tumor sample has the Luminal A molecular subtype; and when it is determined, based on the output of the logistic regression model, that the tumor sample is Ki67 positive, determining that the tumor sample has the Luminal B molecular subtype.

In some embodiments, the fourth set of genes consists of at least some (e.g., 2, 3, 4, 5, or more genes), optionally all, of the following genes: CCNB1, AURKB, AURKA, PLK1, MCM2, BUB1, E2F1, MKI67, MYBL2, and the fifth set of genes consists of at least some, optionally all, of the following genes: GNG12, SOCS5, CRY2, ELN, PTPN21, COL14A1, ZNF608, ZCCHC24, AASS.

In some embodiments, determining the first and second enrichment scores is performed using a single-sample Gene Set Enrichment Analysis (ssGSEA).

Some embodiments further comprise: generating the RNA expression data from sequencing data previously obtained by sequencing the tumor sample obtained from the subject.

In some embodiments, the sequencing data comprises at least 1 million reads, at least 5 million reads, at least 10 million reads, at least 20 million reads, at least 50 million reads, or at least 100 million reads.

In some embodiments, the sequencing data comprises whole exome sequencing (WES) data, bulk RNA sequencing (RNA-seq) data, single cell RNA sequencing (scRNA-seq) data, microarray data, and/or next generation sequencing (NGS) data.

### BRIEF DESCRIPTION OF THE FIGURES

FIG. 1A, FIG. 1B, FIG. 1C, and FIG. 1D are diagrams of illustrative techniques for identifying a molecular subtype of breast cancer for a subject, according to some embodiments of the technology described herein.

FIG. 1E is a block diagram of an example system for identifying a molecular subtype of breast cancer for a subject, according to some embodiments of the technology described herein.

FIG. 2 is a flowchart of an illustrative process for identifying a molecular subtype of breast cancer for a subject, according to some embodiments of the technology described herein.

FIG. 3-1 and FIG. 3-2 show a representative heatmap of 6,223 TCGA and SCAN-B carcinomas segregated into five breast cancer (BC) molecular types (Basal, HER2-high, HER2-low, Luminal A (Lum A), and Luminal B (Lum B) types) by a hierarchical classifier model (e.g., a Light GBM-based classifier).

FIG. 4-1, FIG. 4-2, FIG. 4-3, and FIG. 4-4 show a representative heatmap for molecular BC types developed from the TCGA BRCA, METABRIC, and SCAN-B datasets (n=6,223 samples) with median-scaled expression of PAM50 genes and additional genes used for annotation (*). Genes in bold are BC biomarkers. Gene subsets used for UMAP clustering of each subtype are marked with lines. LumA and LumB subtypes were delineated by low and high proliferation signatures, respectively.

FIGS. 5A-5E show representative data for clinical and genomic features of HER2-low molecular BC type samples. FIG. 5A shows distribution of IHC phenotypes and HER2 FISH positivity across PAM50(BG) subtypes. FIG. 5B shows distribution of luminal androgen receptor (LAR) Burstein subtypes across molecular BC types on METABRIC samples. FIG. 5C shows representative data for overall survival for METABRIC, TCGA, and SCAN-B cohorts stratified by molecular BC types. FIG. 5D shows a representative heatmap with percentages for each driver event (somatic mutation, oncogene amplification, or tumor suppressor deletion) in TCGA and METABRIC samples grouped by molecular BC types. Bar plots represent ratios of the alteration type for each gene in proportion and raw count. FIG. 5E shows percentages of ERBB2 and EGFR alterations across molecular BC types on TCGA and METABRIC cohorts. Adjusted p-values (squares) were derived from the right-tailed Fisher's exact test.

FIGS. 6A-6C show representative data for tumor biology of molecular BC types. FIG. 6A shows distribution of gene expression levels for exemplary BC biomarkers across molecular BC types. FIG. 6B shows distribution of ERBB2 expression levels and copy numbers (annotated by shading) in TCGA BRCA data across molecular BC types. Dashed lines mark previously described HER2-low expression thresholds for IHC. FIG. 6C shows characterization of molecular BC types, including the HER2-low subtype, by proliferation, luminality, basality, and expression of ERBB2 and EGFR, which encode receptor tyrosine kinases.

FIGS. 7A-7J show one embodiment of a process for breast cancer subtype identification. FIG. 7A is a scheme of separation of samples into 5 subtypes. FIG. 7B is a representative UMAP analysis. FIG. 7C shows the reannotation process using density clustering on UMAP. FIG. 7D-1, FIG. 7D-2, FIG. 7D-3, and FIG. 7D-4 show a general heatmap in PAM50 genes with additional genes used for annotation (marked with *). FIG. 7E shows the survival KM curves on Metabric cohort with reported annotation and BG50 (only for main subtypes). FIG. 7F shows the survival KM curves on TCGA cohort with reported annotation and BG50 (only for main subtypes). FIG. 7G shows the survival KM curves on SCAN-B cohort with reported annotation and BG50 (only for main subtypes). FIG. 7H shows the ratio of ERBB2-amplified samples in main subtypes in reported annotation and BG50. FIG. 7I shows the ratio of FISH-positive and negative samples in main subtypes in reported annotation and BG50. FIG. 7J is a confusion matrix between reported annotation and BG50 annotation.

FIGS. 8A-8F is the description of breast cancer subtypes, according to some aspects of the technology described herein. FIG. 8A shows the distribution of IHC phenotypes across BG50 subtypes. FIG. 8B depicts a panorama of driver events (coding mutations, amplifications, deletions) across BG50 subtypes. FIG. 8C shows the alterations (coding mutations, amplifications, deletions) in biomarker genes across BG50 subtypes. FIG. 8D shows the progeny signaling pathways heatmap across BG50 subtypes. FIG. 8E depicts the gene expressions heatmap across BG50 subtypes. Genes were chosen using differential expression. FIG. 8F shows the gene expressions levels (in scaled units) across BG50 subtypes.

FIGS. 9A-9G-3 show data relating to the HER2-low subtype: discrepancies between other subtypes, main properties and drivers. FIG. 9A shows the ratio of FISH-positive and negative samples in BG50. FIG. 9B shows the ratio of ERBB2-amplified samples in BG50. FIG. 9C shows the distribution of Burstein annotation in BG50. FIG. 9D is a representation of alterations in biomarker genes among BG50 subtypes. FIG. 9E is a mutations plot of the ERBB2 gene in the HER2-low subtype. FIG. 9F shows a comparative oncoplot in biomarker genes across HER2-high and HER2-low subtype. FIG. 9G-1, FIG. 9G-2, and FIG. 9G-3 depict the top differentially expressed genes between HER2-low and HER2-high, Luminal, Basal subtypes.

FIGS. 10A-10D show the luminal subtype: rejection of Normal-likes as a PAM50 subtype and development of a new principle for luminal subtype separation. FIG. 10A show slides with samples which are annotated as normal-likes. FIG. 10B show the levels of purity, proliferation, adipose and keratine signatures across BG50 subtypes and normal tissue. FIG. 10C shows the KM survival curves of Luminal A and Luminal B subtypes in previously reported annotation and in BG50. FIG. 10D are barplots illustrating distribution of ki67-positive samples between LumA/LumB subtypes in previously reported annotation and BG50 annotation.

FIGS. 11A-11D show the validation of the BG50 hierarchical classifier on the breast cancer metacohort (15 datasets, 3165 samples). FIG. 11A is a UMAP separation of predicted BG50 subtypes. FIG. 11B is a gene expressions heatmap across BG50 subtypes (based on median z-scores of expressions of biomarker genes). FIG. 11C-1 and FIG. 11C-2 show the distribution of sample characteristics (IHC phenotype, reported grade, BG-predicted grade, TNM stage, N_stage, T_stage) by predicted BG50 subtypes. FIG. 11D show the KM plot with OS for validation dataset.

FIGS. 12A-12K show representative data relating to gene expression analysis of breast cancer subtypes. FIG. 12A is a UMAP plot for basal separation annotated by level of FOXC1 expression. FIG. 12B shows a UMAP plot for HER2-high separation annotated by level of ERBB2 expression. FIG. 12C shows a UMAP plot for HER2-high separation annotated by level of FABP7 expression. FIG. 12D shows a UMAP plot for HER2-low separation annotated by level of EGFR expression. FIG. 12E shows a UMAP plot for HER2-low separation annotated by level of AKR1B15 expression. FIG. 12F shows a UMAP plot for HER2-low separation annotated by level of ACE2 expression. FIG. 12G shows a UMAP plot for HER2-low separation annotated by level of GATA3 expression. FIG. 12H is a heatmap with genes from proliferative and anti-proliferative signature and BG50 LumA/LumB annotation. FIG. 12I shows the ERBB2 expression levels (in log 2 TPM) in Metabric dataset, colored by amplifications presence across BG50 subtypes. FIG. 12J is a heatmap with proliferative, adipose and keratine genes showing that Normal-likes are different from normal tissue but similar to LumA. FIG. 12K shows the COX regression of survival across BG50 annotation+Stage+Grade.

FIGS. 13A-13D show representative data relating to gene expression analysis of breast cancer subtypes. FIG. 13A is a heatmap of median deconvolution scores across BG50 subtypes. FIG. 13B shows the IHC/RNA_cutoffs phenotypes distribution across BG50. FIG. 13C shows the RNAseq cutoffs (TPM in logs): HER2+/ER−: ERBB2>=8.5, ESR<3.5, HER2+/ER+: ERBB2>=8.5, ESR>=3.5, Her2-low: 6<=ERBB2<8.5, HER2−/ER+: ERBB2<6, ESR>=3.5, TNBC: ERBB2<6, ESR<3.5. FIG. 13C show HER2-low IHC subtype predicted by RNA-seq cut-offs. FIG. 13D shows the Burstein annotation in HER2-low subtype (Metabric dataset).

FIG. 13E-1 and FIG. 13E-2 show a general heatmap in PAM50 genes and additional genes used for annotation (marked with *) on validation datasets.

FIG. 14 is a schematic diagram of an illustrative computing system with which aspects described herein may be implemented.

### DETAILED DESCRIPTION

Breast cancer is a highly heterogeneous disease characterized by a diverse range of subtypes, which each comprise different biological features and prognoses. This variability requires tailored therapeutic strategies, which are linked to the biological processes underlying each molecular subtype. For example, human epidermal receptor 2 (HER2) expression status is an important determinant of both prognosis and therapeutic selection (e.g., anti-HER2 agents, for example antibodies and antibody-drug conjugates (ADCs)) in breast cancer patients.

Breast cancer is typically characterized in one of two ways: immunohistochemistry (IHC) phenotyping or molecular subtyping. IHC-based characterization techniques comprise use of antibodies to detect expression of target proteins, for example HER2 (also referred to as ERBB2), estrogen receptor (ER), and/or progesterone receptor (PR), and assign a phenotype score (e.g., HER2 0, +1, +2, +3) to the patient. However, IHC-based characterization has several drawbacks, including a lack of reliability for poorly preserved tissue samples, an observed low diagnostic agreement for HER2 0 and +1 patients, and inability of IHC to account for intra-tumor HER2 heterogeneity.

Molecular subtyping techniques comprise processing gene expression data to identify ‘intrinsic’ subtypes of breast cancer that share common biological processes. Examples of molecular subtyping techniques include Oncotype DX, MammaPrint, and PAM50 (e.g., as described by Parker et al. J Clin Oncol. 2009 Mar. 10; 27(8): 1160-1167). PAM50 is a 50-gene signature that classifies a breast cancer into one of five molecular subtypes: Luminal A, Luminal B, HER2-enriched, Basal-like, and Normal-like. However, despite its clinical assimilation, PAM50-based classification may not account for molecular and biological variability within each of the five intrinsic subtypes.

For example, it has been observed that within breast cancers, a further distinct ‘HER2-low’ molecular subtype may exist (e.g., as described by Nicolo et al. Ther Adv Med Oncol. 2023; 15: 17588359231152842). A HER2-low subtype has also been identified by IHC-based techniques, which characterize this subtype has having HER2 protein expression that falls below HER2+2 and +3 scores but is nonetheless present in tumor cells. The binary HER2-status classification of molecular subtyping techniques such as PAM50 highlights a need for nuanced molecular subtyping methods. Furthermore, HER2-low breast cancers may differ in their responses to therapeutic interventions relative to HER2-negative or HER2-enriched breast cancers. Thus, in therapy-resistant, hormone receptor-negative tumors that have been previously classified as HER2-negative by other methods, molecular subtyping techniques described herein, which can detect HER2-low breast cancer, may identify additional patient sub-populations that are suitable for treatment with HER2-targeting agents.

Aspects of the disclosure relate to methods for identifying molecular types of breast cancer (BC). The disclosure is based, in part, on the recognition that transcriptomic profiling may be used to further subdivide HER2-expressing breast cancers into two subtypes: HER2-low (HER2-low), and HER2-high (HER2-high). In some embodiments, the molecular breast cancer (BC) type of a subject is indicative of one or more characteristics of the subject (or the subject's cancer), for example the likelihood a subject will have a good prognosis or respond to a therapeutic agent such as an anti-HER2 therapy such as Trastuzumab deruxtecan (T-DXd).

In some aspects, the techniques for identifying molecular types of breast cancer include: (a) obtaining RNA expression data specifying RNA expression levels at least for genes in first, second, and third sets of genes; (b) determining, using the RNA expression levels for the first set of genes and a first trained ML classifier, whether the tumor sample has a Basal molecular subtype; (c) when it is determined that the tumor sample does not have the Basal molecular subtype, determining, using the RNA expression levels for the second set of set of genes and a second trained ML classifier, whether the tumor sample has a HER2-high molecular subtype; and (d) when it is determined that the tumor sample does not have the HER2-high molecular subtype, determining, using the RNA expression levels for the third set of set of genes and a third trained ML classifier, whether the tumor sample has a HER2-low molecular subtype. In some embodiments, the molecular subtype identified for the tumor sample is used to identify one or more therapies to be administered or recommended to be administered to the subject.

Following below are descriptions of various concepts related to, and embodiments of, techniques for identifying a molecular subtype of breast cancer for a subject. It should be appreciated that various aspects described herein may be implemented in any of numerous ways, as the techniques are not limited in any particular manner of implementation. Example details of implementations are provided herein solely for illustrative purposes. Furthermore, the techniques disclosed herein may be used individually or in any suitable combination, as aspects of the technology described herein are not limited to the use of any particular technique or combination of techniques.

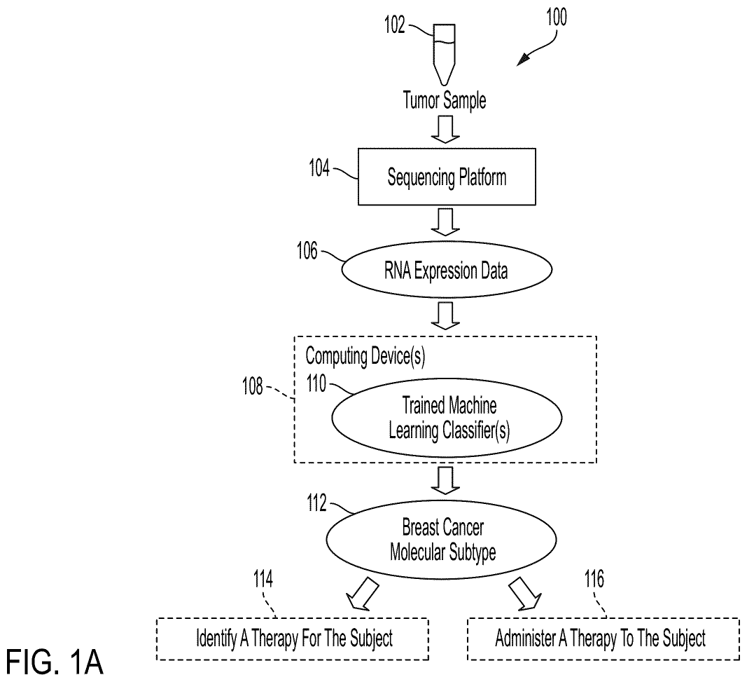

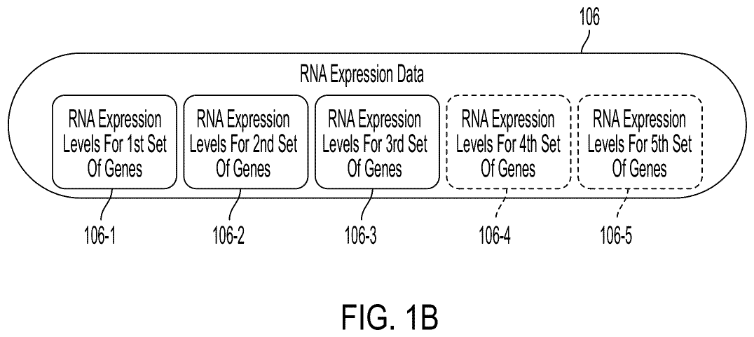

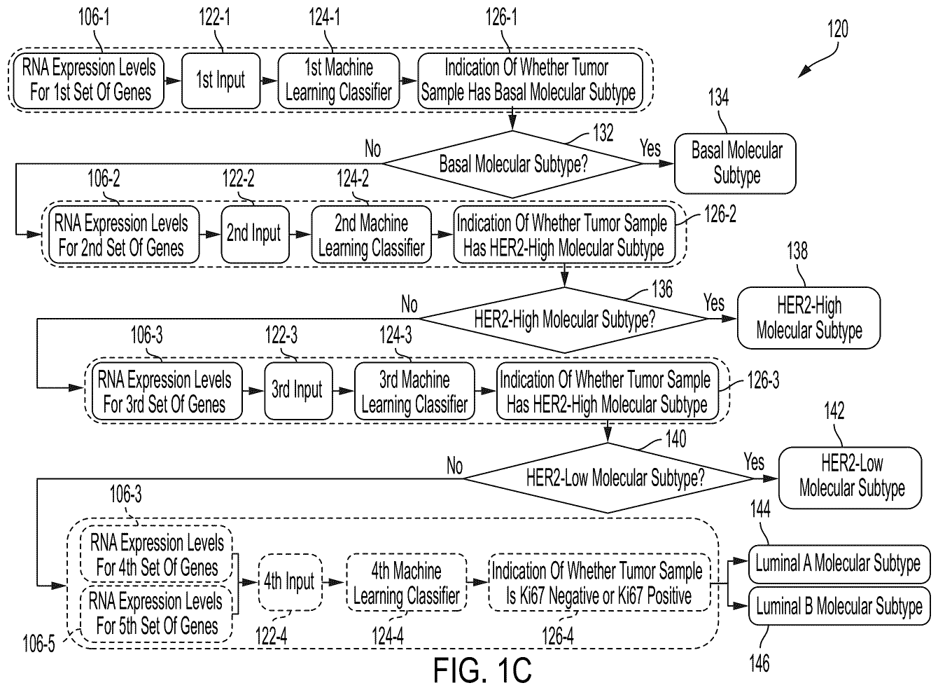

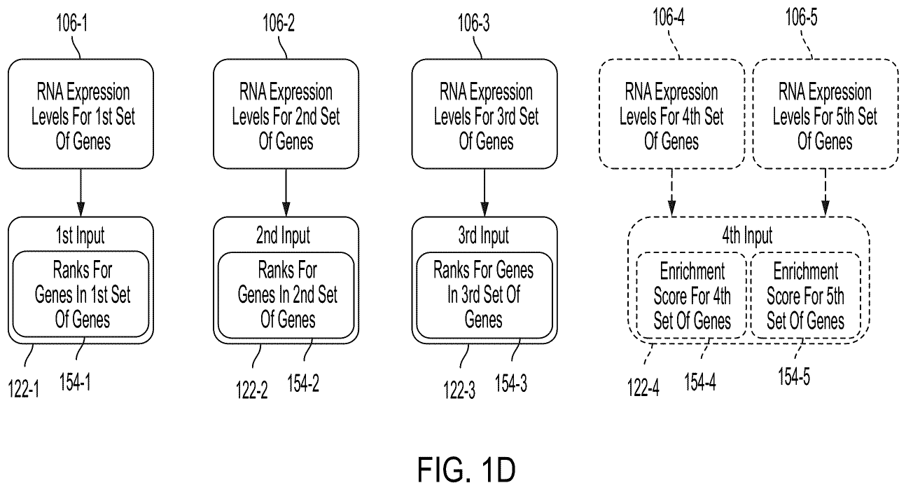

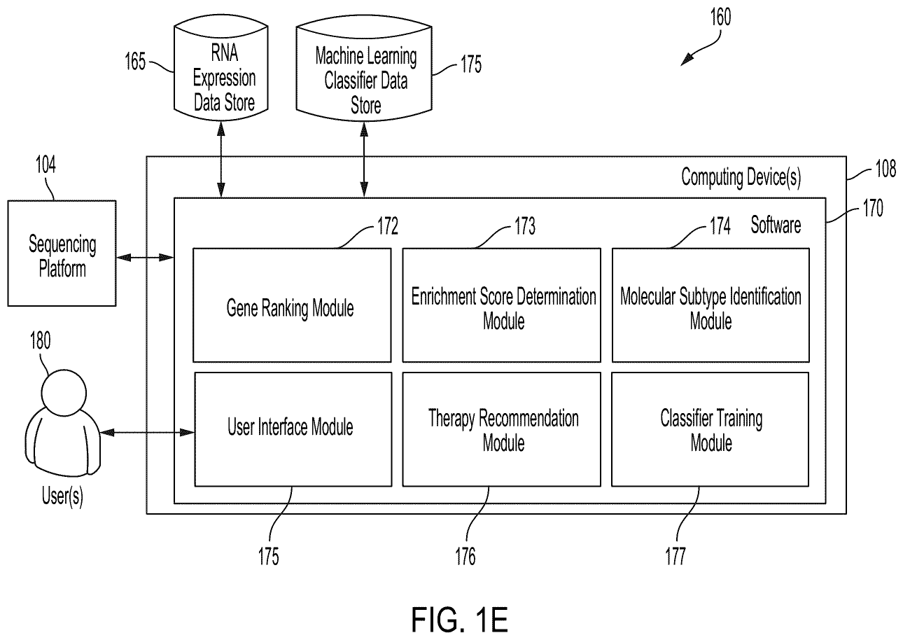

FIG. 1A is a diagram of an illustrative technique 100 for identifying a molecular subtype of breast cancer for a subject, according to some embodiments of the technology described herein. Technique 100 includes (a) obtaining RNA expression data 106 from a tumor sample 102 from a subject (e.g., using sequencing platform 104) and (b) processing the RNA expression data 106 using trained machine learning classifier(s) 110 on computing device(s) 108 to identify a breast cancer molecular subtype 112 for the subject. In some embodiments, the breast cancer molecular subtype 112 is used to identify a therapy for the subject at act 114. In some embodiments, the therapy is administered to the subject at act 116.

In some embodiments, aspects of the illustrative technique 100 are implemented in a clinical or laboratory setting. For example, aspects of the illustrative technique 100 may be implemented on a computing device that is located within the clinical or laboratory setting. For example, the computing device(s) 108 may obtain RNA expression data 106 from a sequencing platform 104 co-located with the computing device(s) 108 within the clinical or laboratory setting. For example, computing device(s) 108 may be included within sequencing platform 104. Additionally, or alternatively, the computing device(s) 108 may indirectly obtain RNA expression data 106 from a sequencing platform 104 that is located externally from or is co-located with the computing device(s) 108 within the clinical or laboratory setting. For example, the computing device(s) 108 may obtain the RNA expression data 106 via at least one communication network, such as the Internet or any other suitable communication network(s), as aspects of the technology described herein are not limited in this respect. Additionally, or alternatively, the computing device(s) 108 may obtain the RNA expression data 106 from a user (e.g., by the user uploading the RNA expression data 106) within the clinical or laboratory setting.

In some embodiments, illustrative technique 100 is implemented in a setting that is located external to a clinical or laboratory setting. In this case, computing device(s) 108 may indirectly obtain the RNA expression data 106 from sequencing platform 104 and/or a computing device located within or external to a clinical or laboratory setting. For example, the RNA expression data 106 may be provided to the computing device(s) 108 via at least one communication network such as the Internet or any other suitable communication network(s), as aspects of the technology described herein are not limited in this respect.

In some embodiments, illustrative technique 100 includes obtaining a tumor sample 102 from a subject. In some embodiments, the subject has, is suspected of having, or is at risk of having cancer. For example, the subject may have breast cancer. In some embodiments, the tumor sample 102 was previously obtained from the subject. A tumor sample, in some embodiments, refers to a sample comprising cells from a tumor. In some embodiments, the tumor sample comprises cells from a benign tumor (e.g., non-cancerous cells). In some embodiments, the tumor sample comprises cells from a premalignant tumor (e.g., precancerous cells). In some embodiments, the tumor sample comprises cells from a malignant tumor (e.g., cancerous cells). The origin, type, and/or preparation of the tumor sample 102 may include any of the embodiments relating to tumor samples described herein including at least in the section entitled “Biological Samples.”

In some embodiments, the RNA expression data 106 includes data obtained from a sequencing platform 104 and/or data derived from data obtained from the sequencing platform 104. In some embodiments, as shown in FIG. 1B, the RNA expression data 106 includes RNA expression levels for one or more genes. In some embodiments, the RNA expression data 106 includes RNA expression levels for at least 15 genes, at least 20 genes, at least 25 genes, at least 50 genes, at least 75 genes, at least 100 genes, at least 150 genes, at least 200 genes, at least 250 genes, at least 500 genes, at least 1,000 genes, at least 1,500 genes, at least 2,000 genes, at least 2,500 genes, at least 3,000 genes, at least 3,500 genes, at least 4,000 genes, at least 4,500 genes, at least 5,000 genes, at least 6000 genes, at least 7,000 genes, at least 8,000 genes, at least 9,000 genes, at least 10,000 genes, at least 15,000 genes, at least 20,000 genes, or at least any other suitable number of genes, as aspects of the technology described herein are not limited in this respect. In some embodiments, the RNA expression data 106 includes RNA expression levels for at most 15 genes, at most 20 genes, at most 25 genes, at most 50 genes, at most 75 genes, at most 100 genes, at most 150 genes, at most 200 genes, at most 250 genes, at most 500 genes, at most 1,000 genes, at most 1,500 genes, at most 2,000 genes, at most 2,500 genes, at most 3,000 genes, at most 3,500 genes, at most 4,000 genes, at most 4,500 genes, at most 5,000 genes, at most 6000 genes, at most 7,000 genes, at most 8,000 genes, at most 9,000 genes, at most 10,000 genes, at most 15,000 genes, at most 20,000 genes, or at most any other suitable number of genes, as aspects of the technology described herein are not limited in this respect. It should be appreciated that any of the above-listed upper bounds may be coupled with any of the above-listed lower bounds. In some embodiments, the RNA expression data 106 includes RNA expression levels for one or more genes included in the PAM50 gene list. For example, genes in the PAM50 gene list may be include those disclosed by Perou, Charles M., et al. (“Molecular portraits of human breast tumours.” nature 406.6797 (2000): 747-752), which is incorporated by reference herein in its entirety. In some embodiments, the RNA expression data 106 includes RNA expression levels for genes in multiple sets of genes. For example, the RNA expression data 106 may include (a) RNA expression levels 106-1 for a first set of genes, (b) RNA expression levels 106-2 for a second set of genes, (c) RNA expression levels 106-3 for a third set of genes, optionally (d) RNA expression levels 106-4 for a fourth set of genes, and optionally (e) RNA expression levels 106-5 for a fifth set of genes. The origin, type, and/or preparation of the RNA expression data 106 may include any of the embodiments described herein including at least in the section entitled “Expression Data.”

In some embodiments, a set of genes for which RNA expression data is obtained is associated with a particular molecular subtype. For example, the first set of genes may be associated with a Basal molecular subtype and may include at least some (e.g., all) of the genes listed for the Basal molecular subtype in Table 1. For example, the first set of genes may include at least 3, at least 4, at least 5, at least 6, at least 7, at least 8, at least 9, at least 10, or all of the genes listed for the Basal molecular subtype in Table 1. The first set of genes may include at most 3, at most 4, at most 5, at most 6, at most 7, at most 8, at most 9, at most 10, or all of the genes listed for the Basal molecular subtype in Table 1. It should be appreciated that any of the above-listed upper bounds may be coupled with any of the above-listed lower bounds. The second set of genes may be associated with a HER2-high molecular subtype and may include at least some (e.g., all) of the genes listed for the HER2-high molecular subtype in Table 1. For example, the second set of genes may include at least 3, at least 4, at least 5, at least 6, at least 7, at least 8, at least 9, at least 10, at least 11, at least 12, at least 15, at least 20, at least 25, at least 30, at least 35, at least 36, at least 37, at least 38, at least 39, or all of the genes listed for the HER2-high molecular subtype in Table 1. The second set of genes may include at most 3, at most 4, at most 5, at most 6, at most 7, at most 8, at most 9, at most 10, at most 11, at most 12, at most 15, at most 20, at most 25, at most 30, at most 35, at most 36, at most 37, at most 38, at most 39, or all of the genes listed for the HER2-high molecular subtype in Table 1. It should be appreciated that any of the above-listed upper bounds may be coupled with any of the above-listed lower bounds. The third set of genes may be associated with a HER2-low molecular subtype and may include at least some (e.g., all) of the genes listed for the HER2-low molecular subtype in Table 1. For example, the third set of genes may include at least 3, at least 4, at least 5, at least 6, at least 7, at least 8, at least 9, at least 10, at least 11, at least 12, at least 15, at least 20, at least 25, at least 30, at least 35, at least 40, at least 45, at least 46, at least 47, or all of the genes listed for the HER2-low molecular subtype in Table 1. The third set of genes may include at most 3, at most 4, at most 5, at most 6, at most 7, at most 8, at most 9, at most 10, at most 11, at most 12, at most 15, at most 20, at most 25, at most 30, at most 35, at most 40, at most 45, at most 46, at most 47, or all of the genes listed for the HER2-low molecular subtype in Table 1. It should be appreciated that any of the above-listed upper bounds may be coupled with any of the above-listed lower bounds. The fourth set of genes may be associated with a Luminal B molecular subtype and may include at least some (e.g., all) of the genes listed for the Luminal B subtype in Table 1. For example, the fourth set of genes may include at least 3, at least 4, at least 5, at least 6, at least 7, at least 8, or all of the genes listed for the Luminal B molecular subtype in Table 1. The fourth set of genes may include at most 3, at most 4, at most 5, at most 6, at most 7, at most 8, or all of the genes listed for the Luminal B molecular subtype in Table 1. It should be appreciated that any of the above-listed upper bounds may be coupled with any of the above-listed lower bounds. The fifth set of genes may be associated with a Luminal A molecular subtype and may include at least some (e.g., all) of the genes listed for the Luminal A subtype in Table 1. For example, the fifth set of genes may include at least 3, at least 4, at least 5, at least 6, at least 7, at least 8, or all of the genes listed for the Luminal A molecular subtype in Table 1. The fifth set of genes may include at most 3, at most 4, at most 5, at most 6, at most 7, at most 8, or all of the genes listed for the Luminal A molecular subtype in Table 1. It should be appreciated that any of the above-listed upper bounds may be coupled with any of the above-listed lower bounds.

TABLE 1

| Molecular Subtype | Genes |
|---|---|
| Basal | FOXA1, MLPH, FOXC1, SFRP1, NAT1, |
| | ORC6, BIRC5, CDC20, AGR2, AR, CA12, |
| | CDK1 |
| HER2-high | MLPH, ESR1, FOXC1, MYC, PHGDH, |
| | ACTR3B, CDH3, KRT14, KRT5, EGFR, |
| | CDC6, FGFR4, MAPT, CXXC5, NAT1, |
| | MDM2, CCNE1, MYBL2, RRM2, NUF2, |
| | TYMS, ANLN, UBE2T, CENPF, PTTG1, |
| | UBE2C, CDC20, MELK, GRB7, ERBB2, |
| | FABP7, GATA3, AR, CCNB2, CDK1, |
| | CLDN8, ERBB3, IGF1R, PIK3CA, TOP2A |
| HER2-low | FOXA1, MLPH, ESR1, MYC, PHGDH, |
| | ACTR3B, SFRP1, KRT17, KRT5, EGFR, |
| | CDC6, FGFR4, MAPT, CXXC5, BCL2, |
| | PGR, NAT1, SLC39A6, BLVRA, CCNE1, |
| | MYBL2, RRM2, ORC6, NUF2, TYMS, |
| | ANLN, CENPF, EXO1, MKI67, BIRC5, |
| | UBE2C, KIF2C, CEP55, MELK, ERBB2, |
| | ACE2, FABP7, AKR1B15, GATA3, AGR3, |
| | AR, CA12, CDK1, CLDN8, E2F2, IGF1R, |
| | SOX11, TFF1, TOP2A |
| Luminal B | CCNB1, AURKB, AURKA, PLK1, MCM2, |
| | BUB1, E2F1, MKI67, MYBL2 |
| Luminal A | GNG12, SOCS5, CRY2, ELN, PTPN21, |
| | COL14A1, ZNF608, ZCCHC24, AASS |

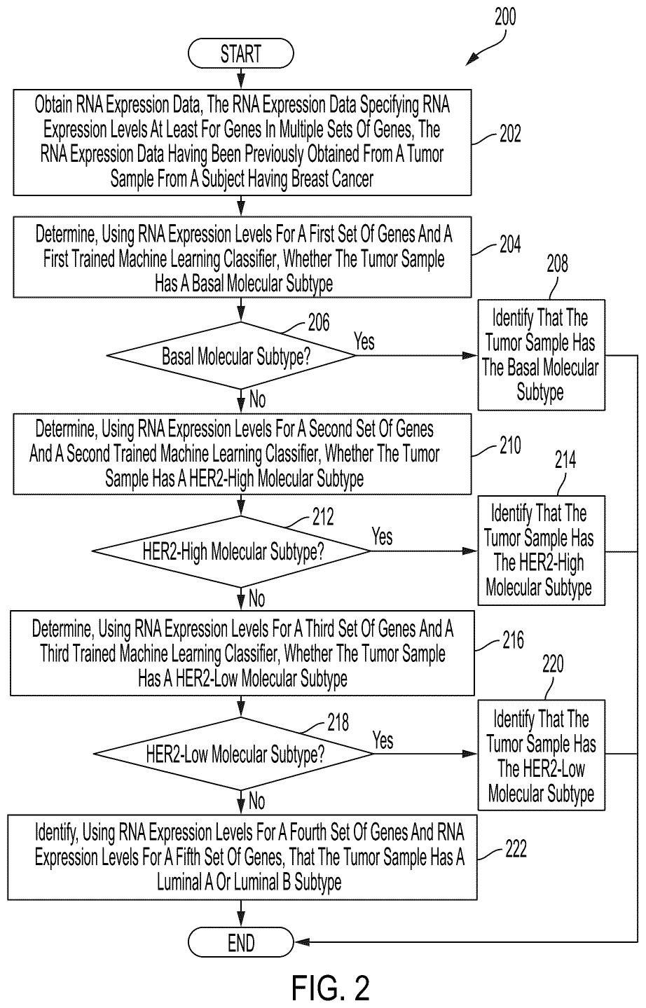

In some embodiments, one or more computing device(s) 108 are used to process the RNA expression data 106 to identify a breast cancer molecular subtype 112 for the subject from which the tumor sample 102 was obtained. For example, software (e.g., software 170 shown in FIG. 1E) on computing device(s) 108 may be configured to process the RNA expression data 106 to identify the breast cancer molecular subtype 112 for the tumor sample 102. In some embodiments, this includes identifying, from among multiple breast cancer molecular subtypes and using the RNA expression data and a plurality trained machine learning classifier(s) 110, a molecular subtype for the tumor sample 102. In some embodiments the multiple breast cancer subtypes include: a Basal subtype, a HER2-high subtype, a HER2-low subtype, a Luminal A subtype, and a Luminal B subtype. Example techniques for identifying a breast cancer molecular subtype for a subject are described herein including at least with respect to FIGS. 1C-1D and process 200 shown in FIG. 2. Examples of breast cancer molecular subtypes are described herein including at least in the section “Molecular Subtypes.”

In some embodiments, the breast cancer molecular subtype 112 is used, at act 114, to identify a therapy for the subject. For example, computing device(s) 108 may be configured to identify the therapy and/or generate an output that includes an indication of the therapy identified for the subject. In some embodiments, a cancer therapy is identified for the subject based on the breast cancer molecular subtype 112. For example, trastuzumab deruxtecan may be identified for the subject when the identified breast cancer molecular subtype 112 is a HER2-low subtype. In some embodiments, the therapy is administered to the subject at act 116. For example, trastuzumab deruxtecan may be administered to the subject when the identified breast cancer molecular subtype 112 is a HER2-low subtype. Examples of therapies and techniques for administering a therapy are described herein including at least in the section entitled “Methods of Treatment.”

In some embodiments, the computing device(s) 108 is configured to generate an output indicating the identified breast cancer molecular subtype 112 identified for the subject and/or a therapy identified for the subject. In some embodiments, the output may be stored (e.g., in memory, in one or more data stores, etc.), displayed via a user interface (e.g., user interface module 175 shown in FIG. 1E), transmitted to one or more other devices, used to generate a report, and/or otherwise processed using any other suitable techniques as aspects of the technology described herein are not limited in this respect. For example, the output of the computing device(s) 108 may be displayed via a graphical user interface (GUI) of a computing device (e.g., computing device(s) 108).

In some embodiments, the output of the computing device(s) 108 may be in the form of a report, such as a report including an indication of the breast cancer molecular subtype 112 and/or a therapy identified for the subject. The generated report can provide a summary of information, so that a clinician can determine whether to administer a therapy to the subject. The report as described herein may be a paper report, an electronic report, or a report in any format that is deemed suitable in the art. The report may be shown and/or stored on a computing device (e.g., computing device(s) 108).

In some embodiments, the methods and reports disclosed herein may include data store management for the keeping of generated reports. For instance, the methods as disclosed herein can create a record in a data store for the subject and populate the specific record with data for the subject. In some embodiments, the generated report can be provided to the subject, clinicians, doctors, researchers, or any other suitable entity, as aspects of the technology described herein are not limited in this respect.

In some embodiments, computing device(s) 108 includes one or multiple computing devices. In some embodiments, when computing device(s) 108 include multiple computing devices, each of the computing devices may be used to perform the same process or processes.

For example, each of the multiple computing devices may include software used to implement process 200 shown in FIG. 2. In some embodiments, when computing device(s) 108 include multiple computing devices, the computing devices are used to perform different processes and/or different acts of a process.

In some embodiments, when computing device(s) 108 include multiple computing devices may be configured to communicate via at least one communication network such as the Internet or any other suitable communication network(s), as aspects of the technology described herein are not limited in this respect. For example, the multiple computing devices may be part of a cloud computing environment. The cloud computing environment may be a public cloud computing environment, a private computing environment or a hybrid computing environment operating using a combination of publicly accessible and private infrastructure.

FIG. 1C is a diagram of an illustrative technique 120 for identifying a breast cancer molecular subtype for a subject, according to some embodiments of the technology described herein. In some embodiments, the technique 120 includes processing at least some of the RNA expression data 106 using one or more of multiple trained machine learning classifiers 110-1, 110-2, 110-3, and 110-4 to identify a molecular subtype from among a Basal molecular subtype 134, a HER2-high molecular subtype 138, a HER2-low molecular subtype 142, a Luminal A molecular subtype 144, and a Luminal B molecular subtype 146.

In some embodiments, illustrative technique 120 includes using the RNA expression levels 106-1 for the first set of genes and a first trained machine learning classifier 110-1 to determine whether the tumor sample has a Basal molecular subtype. In some embodiments, the RNA expression levels 106-1 are used to generate first input 122-1 for the first machine learning classifier 110-1.

In some embodiments, with reference to FIG. 1D, generating the first input 122-1 includes determining ranks 154-1 for at least some e.g., all) genes in the first set of genes. For example, ranks may be determined for at least 3, at least 4, at least 5, at least 6, at least 7, at least 8, at least 9, at least 10, or all of the genes listed for the Basal molecular subtype in Table 1. The ranks maybe determined for at most 3, at most 4, at most 5, at most 6, at most 7, at most 8, at most 9, at most 10, or all of the genes listed for the Basal molecular subtype in Table 1. It should be appreciated that any of the above-listed upper bounds may be coupled with any of the above-listed lower bounds.

In some embodiments, the ranks 154-1 include values (e.g., integers) identifying relative ranks of at least some genes in the first set of genes. The ranks for the genes may be in ascending or descending order. In some embodiments, the values are different from the RNA expression levels 106-1. For example, genes [A, B, C], having respective expression levels of 0.01, 0.56, and 0.45 would be ranked [1, 3, 2] if they were to be ranked in ascending order. In some embodiments, a gene ranking may be stored (and/or manipulated in a computer) using at least one data structure having fields storing gene ranking values. Example techniques for ranking genes are described in U.S. patent application Ser. No. 17/113,008, titled “MACHINE LEARNING TECHNIQUES FOR GENE EXPRESSION ANALYSIS”, filed on Dec. 5, 2020, which is incorporated by reference herein in its entirety.

In some embodiments, the ranks are determined relative to genes included in the first set of genes. In this instance, if the first set of genes includes ten genes, the ten ranks for the ten genes may range from 1-10. In some embodiments, the ranks are determined relative to genes included in the first set of genes and one or more additional genes for which RNA expression data 106 was obtained. For example, the ranks may be determined relative to all genes that were sequenced to generate expression data 106. In this instance, if the first set of genes includes ten genes, the ten ranks for the ten genes may range anywhere from 1 to the total number of genes sequenced.

In some embodiments, the first input 122-1 includes a vector of the ranks 154-1 determined for the genes in the first set of genes. For example, each vector component may be a value identifying the rank of a respective gene in the first set of genes.

In some embodiments, the first machine learning classifier 110-1 is trained to predict whether the tumor sample has a Basal molecular subtype given ranks 154-1 and/or RNA expression levels 106-1 as input. The first trained machine learning classifier 110-1 may include any suitable type of machine learning classifier such as, for example, a gradient-boosted decision tree model, a generalized linear model (e.g., a logistic regression model, a probit regression model, etc.), a support vector machine, a decision tree model, a Gaussian mixture model, a random forest model, a neural network model, or any other suitable type of machine learning classifier, as aspects of the technology described herein are not limited in this respect.

In some embodiments, the first trained machine learning classifier 110-1 is trained using a plurality of training samples, each of which includes (a) ranks and/or RNA expression levels for at least some genes included in the first set of genes, and (b) an indication of whether or not the sample has the Basal molecular subtype. The first machine learning classifier 110-1 may be trained using any suitable training techniques including any of those described herein including at least in the section entitled “Machine Learning.” In some embodiments, the output of the first trained machine learning classifier 110-1 includes an indication 126-1 of whether the tumor sample has the Basal molecular subtype. The output may include a binary output, a probability, an output identifying one of multiple classes, or any other suitable type of output, as aspects of the technology described herein are not limited in this respect.

In some embodiments, at act 132, if the tumor sample is determined not to have the Basal molecular subtype, then illustrative technique 120 includes using the RNA expression levels 106-2 for the second set of genes and a second trained machine learning classifier 110-2 to determine whether the tumor sample has a HER2-high molecular subtype. In some embodiments, the RNA expression levels 106-2 are used to generate second input 122-2 for the second machine learning classifier 110-2.

In some embodiments, with reference to FIG. 1D, generating the second input 122-2 includes using the RNA expression levels 106-2 to determine ranks 154-2 for at least some (e.g., all) genes in the second set of genes. For example, ranks may be determined for at least 3, at least 4, at least 5, at least 6, at least 7, at least 8, at least 9, at least 10, at least 11, at least 12, at least 15, at least 20, at least 25, at least 30, at least 35, at least 36, at least 37, at least 38, at least 39, or all of the genes listed for the HER2-high molecular subtype in Table 1. The ranks may determined for at most 3, at most 4, at most 5, at most 6, at most 7, at most 8, at most 9, at most 10, at most 11, at most 12, at most 15, at most 20, at most 25, at most 30, at most 35, at most 36, at most 37, at most 38, at most 39, or all of the genes listed for the HER2-high molecular subtype in Table 1. It should be appreciated that any of the above-listed upper bounds may be coupled with any of the above-listed lower bounds.

In some embodiments the ranks 154-2 includes values (e.g., integers) identifying relative ranks of at least some genes in the second set of genes. The ranks for the genes may be in ascending or descending order. In some embodiments, the values are different from the RNA expression levels 106-2. In some embodiments, the gene ranking may be stored (and/or manipulated in a computer) using at least one data structure having fields storing gene ranking values.

In some embodiments, the ranks are determined relative to genes included in the second set of genes. In some embodiments, the ranks are determined relative to genes in the second set of genes and one or more additional genes for which RNA expression data 106 was obtained. For example, the ranks may be determined relative to all genes that were sequenced to generate expression data 106.

In some embodiments, the second input 122-2 includes a vector of the ranks 154-2 determined for the genes in the second set of genes. For example, each vector component may be a value identifying the rank of a respective gene in the second set of genes.

In some embodiments, the second machine learning classifier 110-2 is trained to predict whether the tumor sample has a HER2-high molecular subtype given ranks 154-2 and/or RNA expression levels 106-2 as input. The second trained machine learning classifier 110-2 may include any suitable type of machine learning classifier such as, for example, a gradient-boosted decision tree model, a generalized linear model (e.g., a logistic regression model, a probit regression model, etc.), a support vector machine, a decision tree model, a Gaussian mixture model, a random forest model, a neural network model, or any other suitable type of machine learning classifier, as aspects of the technology described herein are not limited in this respect. In some embodiments, the second trained machine learning classifier 110-2 is trained using a plurality of training samples, each of which includes (a) ranks and/or RNA expression levels for at least some genes included in the second set of genes, and (b) an indication of whether or not the sample has the HER2-high molecular subtype. The second machine learning classifier 110-2 may be trained using any suitable training techniques including any of those described herein including at least in the section entitled “Machine Learning.”

In some embodiments, the output of the second trained machine learning classifier 110-2 includes an indication 126-2 of whether the tumor sample has the HER2-high molecular subtype. The output may include a binary output, a probability, an output identifying one of multiple classes, or any other suitable type of output, as aspects of the technology described herein are not limited in this respect.

In some embodiments, at act 136, if the tumor sample is determined not to have the HER2-high molecular subtype, then illustrative technique 120 includes using the RNA expression levels 106-3 for the third set of genes and a third trained machine learning classifier 110-3 to determine whether the tumor sample has a HER2-low molecular subtype. In some embodiments, the RNA expression levels 106-3 are used to generate third input 122-3 for the third machine learning classifier 110-3.

In some embodiments, with reference to FIG. 1D, generating the third input 122-3 includes using the RNA expression levels 106-3 to determine ranks 154-3 for at least some (e.g., all) genes in the third set of genes. For example, ranks may be determined for at least 3, at least 4, at least 5, at least 6, at least 7, at least 8, at least 9, at least 10, at least 11, at least 12, at least 15, at least 20, at least 25, at least 30, at least 35, at least 40, at least 45, at least 46, at least 47, or all of the genes listed for the HER2-low molecular subtype in Table 1. Ranks may be determined for at most 3, at most 4, at most 5, at most 6, at most 7, at most 8, at most 9, at most 10, at most 11, at most 12, at most 15, at most 20, at most 25, at most 30, at most 35, at most 40, at most 45, at most 46, at most 47, or all of the genes listed for the HER2-low molecular subtype in Table 1. It should be appreciated that any of the above-listed upper bounds may be coupled with any of the above-listed lower bounds.

In some embodiments the ranks 154-3 includes values (e.g., integers) identifying relative ranks of at least some genes in the third set of genes. The ranks for the genes may be in ascending or descending order. In some embodiments, the values are different from the RNA expression levels 106-3. In some embodiments, the gene ranking may be stored (and/or manipulated in a computer) using at least one data structure having fields storing gene ranking values.

In some embodiments, the ranks are determined relative to only genes included in the third set of genes. In some embodiments, the ranks are determined relative to genes in the third set of genes and one or more additional genes for which RNA expression data 106 was obtained. For example, the ranks may be determined relative to all genes that were sequenced to generate expression data 106.

In some embodiments, the third input 122-3 includes a vector of the ranks 154-3 determined for the genes in the third set of genes. For example, each vector component may be a value identifying the rank of a respective gene in the third set of genes.

In some embodiments, the third machine learning classifier 110-3 is trained to predict whether the tumor sample has a HER2-low molecular subtype given ranks 154-3 and/or RNA expression levels 106-3 as input. The third trained machine learning classifier 110-3 may include any suitable type of machine learning classifier such as, for example, a gradient-boosted decision tree model, a generalized linear model (e.g., a logistic regression model, a probit regression model, etc.), a support vector machine, a decision tree model, a Gaussian mixture model, a random forest model, a neural network model, or any other suitable type of machine learning classifier, as aspects of the technology described herein are not limited in this respect.

In some embodiments, the third trained machine learning classifier 110-3 is trained using a plurality of training samples, each of which includes (a) ranks and/or RNA expression levels for at least some genes included in the third set of genes, and (b) an indication of whether or not the sample has the HER2-low molecular subtype. The third machine learning classifier 110-3 may be trained using any suitable training techniques including any of those described herein including at least in the section entitled “Machine Learning.” In some embodiments, the output of the third trained machine learning classifier 110-3 includes an indication 126-3 of whether the tumor sample has the HER2-low molecular subtype.

The output may include a binary output, a probability, an output identifying one of multiple classes, or any other suitable type of output, as aspects of the technology described herein are not limited in this respect.

In some embodiments, at act 140, if the tumor sample is determined not to have the HER2-low molecular subtype, then illustrative technique 120 includes using the RNA expression levels 106-4 for the fourth set of genes and/or RNA expression levels 106-5 for the fifth set of genes to determine whether the tumor sample has a Luminal A 144 or Luminal B 146 molecular subtype. In some embodiments, RNA expression levels 106-4 and RNA expression levels 106-5 are used to generate the fourth input 122-4 for the fourth machine learning classifier 110-4.

In some embodiments, with reference to FIG. 1D, generating the fourth input 122-4 includes determining an enrichment score 154-4 for at least some (e.g., all) genes included in the fourth set of genes. For example, an enrichment score may be determined for at least 3, at least 4, at least 5, at least 6, at least 7, at least 8, or all of the genes listed for the Luminal B molecular subtype in Table 1. An enrichment score may be determined for at most 3, at most 4, at most 5, at most 6, at most 7, at most 8, or all of the genes listed for the Luminal B molecular subtype in Table 1. It should be appreciated that any of the above-listed upper bounds may be coupled with any of the above-listed lower bounds.

Additionally, or alternatively, generating the fourth input 122-4 may include determining an enrichment score 154-5 for at least some (e.g., all) genes included in the fifth set of genes. For example, an enrichment score may be determined for at least 3, at least 4, at least 5, at least 6, at least 7, at least 8, or all of the genes listed for the Luminal A molecular subtype in Table 1. An enrichment score may be determined for at most 3, at most 4, at most 5, at most 6, at most 7, at most 8, or all of the genes listed for the Luminal A molecular subtype in Table 1. It should be appreciated that any of the above-listed upper bounds may be coupled with any of the above-listed lower bounds.

In some embodiments, the enrichment scores 154-4 and 154-5 are determined by performing single sample Gene Score Enrichment Analysis (ssGSEA) using the RNA expression levels 106-4 and RNA expression levels 106-5. Techniques for performing GSEA are described herein including at least in the section entitled “Expression Data.” In some embodiments, the fourth input 122-4 includes (a) the enrichment score 154-4 determined for genes in the fourth set of genes, and (b) the enrichment score 154-5 determined for genes in the fifth set of genes.

The fourth trained machine learning classifier 110-4 may include any suitable type of machine learning classifier such as, for example, a gradient-boosted decision tree model, a generalized linear model (e.g., a logistic regression model, a probit regression model, etc.), a support vector machine, a decision tree model, a Gaussian mixture model, a random forest model, a neural network model, or any other suitable type of machine learning classifier, as aspects of the technology described herein are not limited in this respect. In some embodiments, the fourth trained machine learning classifier 110-4 is trained using a plurality of training samples, each of which includes (a) enrichment scores determined for the fourth and fifth sets of genes and/or RNA expression levels for the fourth and fifth sets of genes, and (b) an indication of whether or not the sample is Ki67 positive or Ki67 negative, or whether the sample has a Luminal A or Luminal B subtype. The fourth machine learning classifier 110-4 may be trained using any suitable training techniques including any of those described herein including at least in the section entitled “Machine Learning.”

In some embodiments, the output of the fourth trained machine learning classifier 110-4 includes an indication 126-4 of whether the tumor sample is Ki67 positive or Ki67 negative. In some embodiments, the output includes an indication (not shown) of whether the tumor sample has a Luminal A or Luminal B molecular subtype. The output may include a binary output, a probability, an output identifying one of multiple classes, or any other suitable type of output, as aspects of the technology described herein are not limited in this respect.

In some embodiments, when the output of the fourth trained machine learning classifier 110-4 is an indication 126-4 of whether the tumor sample is Ki67 positive or negative, the indication 126-4 may be used to determine whether the tumor sample has a Luminal A molecular subtype 144 or a Luminal B molecular subtype 146. For example, if the tumor sample is determined to be Ki67 positive, then the Luminal B molecular subtype 146 may be identified for the tumor sample. If the tumor sample is determined to be Ki67 negative, then the Luminal A molecular subtype 146 may be identified for the tumor sample.

FIG. 1E is a block diagram of an example system 160 for identifying a molecular subtype of breast cancer for a subject, according to some embodiments of the technology described herein. System 160 includes computing device(s) 108 configured to have software 170 execute thereon to perform various functions in connection with identifying a molecular subtype of breast cancer for a subject. In some embodiments, software 170 includes a plurality of modules. A module may include processor-executable instructions that, when executed by at least one computer hardware processor, cause the at least one computer hardware processor to perform the function(s) of the module. Such modules are sometimes referred to herein as “software modules,” each of which includes processor executable instructions configured to perform one or more processes, such as process 200 described herein including at least with respect to FIG. 2.

The computing device(s) 108 may be operated by one or more user(s) 180. For example, the user(s) 180 may include one or more individuals who are treating and/or studying (e.g., doctors, clinicians, researchers, etc.) the subject. Additionally, or alternatively, user(s) 180 may include the subject. In some embodiments, the user(s) 180 provides input to computing device(s) 108. For example, user(s) 180 may provide, as input to the computing device(s) 108, RNA expression data having been previously obtained from one or more tumor samples from one or more subjects. Additionally, or alternatively, user(s) 180 may provide input specifying processing or other methods to be performed on RNA expression data. Additionally, or alternatively, the user(s) 180 may access results of processing the RNA expression data. For example, the user(s) 180 may access a breast cancer molecular subtype and/or a therapy identified for a subject. User(s) 180 may provide input by uploading one or more files, interacting with a user interface, or using any other suitable technique for providing input, as aspects of the technology described herein are not limited in this respect.

In some embodiments, the gene ranking module 172 is configured to rank genes given the RNA expression levels of the genes. For example, with reference to FIGS. 1B-1D, the gene ranking module 172 may be configured to rank at least some of the genes in the first, second, and/or third set of genes given RNA expression levels 106-1, 106-2, and/or 106-3. In some embodiments, the gene ranking module 172 is configured to rank genes in an ascending or descending order based upon the gene expression levels. The gene ranking module 172 may be configured to implement any of the techniques for ranking genes described in U.S. patent application Ser. No. 17/113,008, titled “MACHINE LEARNING TECHNIQUES FOR GENE EXPRESSION ANALYSIS”, filed on Dec. 5, 2020, which is incorporated by reference herein in its entirety.

In some embodiments, the enrichment score determination module 173 is configured to determine an enrichment score for each of one or more sets of genes. For example, with reference to FIGS. 1B-1D, the enrichment score determination module 175 may be configured to determine enrichment scores for the fourth and fifth sets of genes given RNA expression levels 106-4 and 106-5 for the fourth and fifth sets of genes. The enrichment score determination module 173 may be configured to determine the enrichment scores by performing GSEA (e.g., ssGSEA). For example, the enrichment score determination module 173 may be configured to implement any of the techniques for performing GSEA described by Subramanian, Aravind, et al. (“Gene set enrichment analysis: a knowledge-based approach for interpreting genome-wide expression profiles.” Proceedings of the National Academy of Sciences 102.43 (2005): 15545-15550), which is incorporated by reference herein in its entirety.

In some embodiments, the molecular subtype identification module 174 is configured to identify a breast cancer molecular subtype for a subject using one or more trained machine learning classifiers. For example, the molecular subtype identification module 174 may be configured to identify the molecular subtype using one or more of: (a) a first machine learning classifier trained to predict whether a tumor sample has a Basal molecular subtype, (b) a second machine learning classifier trained to predict whether the tumor sample has a HER2-high molecular subtype, (c) a third machine learning classifier trained to predict whether the tumor sample has a HER2-low molecular subtype, and (d) a fourth machine learning classifier trained to predict either (i) whether the tumor sample is Ki67 positive or Ki67 negative or (ii) whether the tumor sample has a Luminal A or Luminal B molecular subtype. In some embodiments, the molecular subtype identification module 174 is configured to provide input to the particular machine learning classifier(s) used to identify the molecular subtype for the subject. For example, the molecular subtype identification module 174 may be configured to provide, as input to a particular classifier, gene ranks (e.g., a vector of gene ranks) for a set of genes, gene enrichment score(s) for one or more sets of genes, and/or RNA expression levels for genes in a set of genes. In some embodiments, the molecular subtype identification module 174 is configured to obtain, as output from the trained classifier(s), indication(s) as to whether the tumor sample has particular breast cancer molecular subtype(s).

In some embodiments, the therapy recommendation module 176 is configured to identify a therapy to be administered to the subject. In some embodiments, the therapy recommendation module 176 is configured to identify the therapy based on the molecular subtype identified for the subject. For example, the therapy recommendation module 176 may be configured to identify a cancer therapy for the subject based upon the breast cancer molecular subtype identified for the subject.

In some embodiments, the classifier training module 177 is configured to train one or more machine learning classifiers to predict whether the subject has a particular breast cancer molecular subtype. For example, the classifier training module 177 may obtain training data and/or validation data (e.g., from RNA expression data store 165, sequencing platform 104, user(s) 180, etc.). The training and/or validation data may include, for each of a plurality of samples, RNA expression levels for genes, ranks for genes, enrichment score(s) for set(s) of genes, and or a label identifying the molecular subtypes of the particular sample. The classifier training module 177 may be configured to use the obtained training and/or validation data to train and/or validate the machine learning classifier(s) to predict whether a subject has a particular molecular type. Example techniques for training machine learning classifiers are described herein including at least in the section entitled “Machine Learning.” In some embodiments, the classifier training module 177 provides the trained classifier(s) to machine learning classifier data store 175 for storage thereon. For example, the classifier training module 177 may provide the values of parameters of the machine learning classifier(s) to machine learning classifier data store 175 for storage thereon.

As shown in FIG. 1E, software 170 also includes user interface module 175. User interface module 175 may be configured to generate a graphical user interface (GUI) through which user(s) 180 may provide input and view information generated by software 170. For example, in some embodiments, the user interface module 175 may be a webpage or web application accessible through an Internet browser. In some embodiments, the user interface module 175 may generate a GUI of an app executing on a user's mobile device. In some embodiments, the user interface module 175 may generate a number of selectable elements through which a user may interact. For example, the user interface module 175 may generate dropdown lists, checkboxes, text fields, or any other suitable elements.

In some embodiments, the user interface module 175 is configured to generate a GUI including one or more results of identifying a breast cancer molecular subtype for a subject. For example, the GUI may include an indication of the molecular subtype identified for the subject. Additionally, or alternatively, the GUI may include an indication of one or more therapies recommended for administration to the subject. It should be appreciated that the GUI may include any other suitable information, as aspects of the technology described herein are not limited in this respect.

In some embodiments, the RNA expression data store 165 stores at least some of the RNA expression data 106 shown in FIGS. 1A-1D. In some embodiments, RNA expression data store 165 stores training data used to train one or more machine learning classifiers to determine whether a tumor sample has a particular breast cancer molecular subtype. For example, the training data may include RNA expression data for one or more training sample and label(s) identifying breast cancer molecular subtypes for the training sample(s). In some embodiments, the RNA expression data store 165 stores intermediate results of processing the RNA expression data. For example, the RNA expression data store 165 may store gene ranks (e.g., as determined by gene ranking module 172) and/or enrichment scores (e.g., as determined by enrichment score determination module 173). It should be appreciated, however, that RNA expression data store 165 may store any other suitable information, as aspects of the technology described herein are not limited in this respect. In some embodiments, the RNA expression data store 165 includes any suitable type of data store (e.g., a flat file, a database system, a multi-file, etc.) and may store data in any suitable format, as aspects of the technology described herein are not limited in this respect. The RNA expression data store 165 may be part of software 170 (not shown) or excluded from software 170, as shown in FIG. 1E.

In some embodiments, machine learning classifier data store 175 stores one or more trained machine learning classifiers. For example, the machine learning classifier data store 175 may store parameters of one or more trained machine learning classifiers. With reference to FIG. 1C, machine learning classifier data store 175 may store parameters of one or more of the first trained machine learning classifier 110-1, the second trained machine learning classifier 110-2, the third trained machine learning classifier 110-3, and the fourth trained machine learning classifier 110-4. In some embodiments, the machine learning classifier data store 175 includes any suitable type of data store (e.g., a flat file, a database system, a multi-file, etc.) and may store data in any suitable format, as aspects of the technology described herein are not limited in this respect. The machine learning classifier data store 175 may be part of software 170 (not shown) or excluded from software 170, as shown in FIG. 1E.

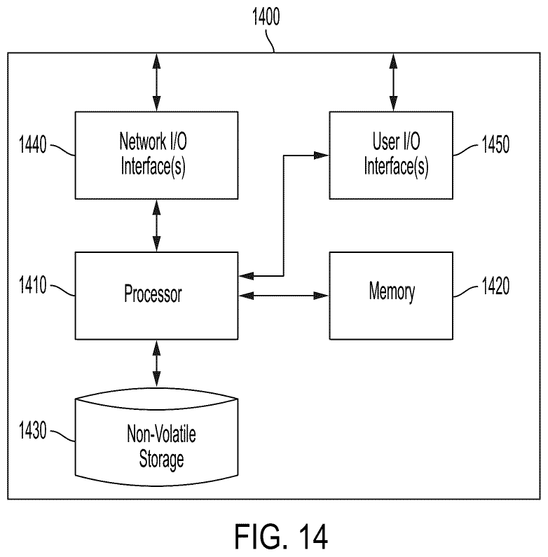

FIG. 2 is a flowchart of an illustrative process 200 for identifying a molecular subtype of breast cancer for a subject, according to some embodiments of the technology described herein. Process 200 may be performed by a laptop computer, a desktop computer, one or more servers, on a mobile device, in a cloud computing environment, computing device(s) 108 shown in FIG. 1A and FIG. 1E, computing system 1400 shown in FIG. 14, or any other suitable computing device(s), as aspects of the technology described herein are not limited in this respect.

At act 202, RNA expression data is obtained. In some embodiments, the RNA expression data was previously obtained from a tumor sample from a subject having breast cancer. In some embodiments, the RNA expression data specifies RNA expression levels genes in multiple sets of genes. For example, the RNA expression data may specify RNA expression levels for at least some genes in a first set of genes, a second set of genes, a third set of genes, a fourth set of genes, and/or a fifth set of genes. Example techniques for obtaining RNA expression data are described herein including at least with respect to FIG. 1A and FIG. 1B.

At act 204, RNA expression levels for a first set of genes and a first trained machine learning classifier are used to determine whether the tumor sample has a Basal molecular subtype. Example techniques for determining whether the tumor sample has a Basal molecular subtype are described herein including at least with respect to FIG. 1C.

If the tumor sample is determined to have the Basal molecular subtype then, at act 206, process 200 proceeds to act 208. At act 208, the tumor sample is identified as having the basal molecular subtype.

If the tumor sample is determined to not have the Basal molecular subtype then, at act 206, process 200 proceeds to act 210. At act 210, RNA expression levels for a second set of genes and a second trained machine learning classifier are used to determine whether the tumor sample has a HER2-high molecular subtype. Example techniques for determining whether the tumor sample has a HER2-high molecular subtype are described herein including at least with respect to FIG. 1C.

If the tumor sample is determined to have a HER2-high molecular subtype then, at act 212, process 200 proceeds to act 214. At act 214, the tumor sample is identified as having the HER2-high molecular subtype.

If the tumor sample is determined to not have a HER2-high molecular subtype then, at act 212, process 200 proceeds to act 216. At act 216, RNA expression levels for a third set of genes and a third trained machine learning classifier are used to determine whether the tumor sample has HER2-low molecular subtype. Example techniques for determining whether the tumor sample has a HER2-low molecular subtype are described herein including at least with respect to FIG. 1C.

If the tumor sample is determined to have a HER2-low molecular subtype then, at act 218, process 200 proceeds to act 220. At act 220, the tumor sample is identified as having the HER2-low molecular subtype.

If the tumor sample is determined to not have a HER2-low molecular subtype then, at act 218, process 200 proceeds to act 222. At act 222, RNA expression levels for a fourth set of genes and RNA expression levels for a fifth set of genes are used to identify that the tumor sample has a Luminal A or Luminal B molecular subtype. Example techniques for determining whether the tumor sample has a Luminal A or Luminal B molecular subtype are described herein including at least with respect to FIG. 1C.

It should be appreciated that process 200 may include one or more additional or alternative acts. For example, process 200 may include all or a subset of all of acts 202, 204, 206, 208, 210, 212, 214, 216, 218, 220, and 222. A subset of the acts may include: acts 202, 204, 206, 208, 210, 212, 214, 216, 218, and 220 or acts 202, 204, 206, 208, 210, 212, and 214.

### Breast Cancer

Aspects of the disclosure relate to methods of determining the breast cancer molecular subtype for a subject having, suspected of having, or at risk of having breast cancer. As used herein, a subject may be a mammal, for example a human, non-human primate, rodent (e.g., rat, mouse, guinea pig, etc.), dog, cat, horse etc. In some embodiments, the subject is a human. The terms “individual” or “subject” may be used interchangeably with “patient.” As used herein, “breast cancer” or “BC” refers to any breast cancer, for example, ductal carcinoma in situ, invasive ductal carcinoma, inflammatory breast cancer, and metastatic breast cancer, or any other type of malignancy caused by one or more various genetic mutations in the body that affect cells (originally present in or metastasized to) the breast and/or tissue surrounding the breast of a subject. As used herein, “cancer” refers to any malignant and/or invasive growth or tumor caused by abnormal cell growth in a subject, including solid tumors, blood cancer, bone marrow or lymphoid cancer, etc. In some embodiments, a breast cancer is a basal-like breast cancer (BLBC), luminal breast cancer (including luminal A and luminal B or normal-like breast cancer; LNLBC), HER2-high breast cancer, or HER2-low breast cancer.

A subject having BC may exhibit one or more signs or symptoms of BC, for example the presence of cancerous cells (e.g., tumor cells), lumps on the breast, fever, swelling, bleeding, nausea and vomiting, and weight loss. In some embodiments, a subject having BC does not exhibit one or more signs or symptoms of BC. In some embodiments, a subject having BC has been diagnosed by a medical professional (e.g., a licensed physician) as having BC based upon one or more assays (e.g., clinical assays, molecular diagnostics, etc.) that indicate that the subject has BC, even in the absence of one or more signs or symptoms. In some embodiments, the intrinsic molecular subtype of a BC subject has been determined.

A subject suspected of having BC typically exhibits one or more signs or symptoms of BC. In some embodiments, a subject suspected of having BC exhibits one or more signs or symptoms of BC but has not been diagnosed by a medical professional (e.g., a licensed physician) and/or has not received a test result (e.g., a clinical assay, molecular diagnostic, etc.) indicating that the subject has BC.

A subject at risk of having BC may or may not exhibit one or more signs or symptoms of BC. In some embodiments, a subject at risk of having BC comprises one or more risk factors that increase the likelihood that the subject will develop BC. Examples of risk factors include the presence of pre-cancerous cells in a clinical sample, having one or more genetic mutations that predispose the subject to developing cancer (e.g., BC), taking one or more medications that increase the likelihood that the subject will develop cancer (e.g., BC), family history of BC, and the like.

### Breast Cancer Molecular Subtypes

Aspects of the disclosure relate to identifying a molecular subtype of breast cancer for a subject using RNA expression data from a tumor sample from the subject. In some embodiments, the breast cancer molecular subtypes include a Basal molecular subtype, a HER2-high molecular subtype, a HER2-low molecular subtype, a Luminal A molecular subtype, and a Luminal B molecular subtype.

In some embodiments, breast cancer molecular subtypes are characterized by patterns of alterations across groups of genes. For example, breast cancer molecular subtypes may be characterized by events (e.g., mutations, deep deletions, and/or amplifications) associated with oncogenic suppressor genes (e.g., TP53, BRCA1, PTEN, CDH1, RB1, ARID1A, MAP3K1, FAT3, KMT2C, KMT2D, FBXW7, CREBBP, MAP2K4, etc.) and/or oncogenes (e.g., PIK3CA, ERBB2, AKT1, GATA3, SF3B1, CCNE2, FGFR1, TOP2A, EGFR, FGFR2, MET, etc.).

Tables 2-1 and 2-2 list representative statistics for each breast cancer molecular subtype as determined by methods described herein. For each breast cancer molecular subtype, Table 2-1 lists the percentage of subjects having the breast cancer molecular subtype that have a particular event associated with a particular gene. The events include mutation coding (M), germline mutation (G), amplification (AA), and deep deletion (DD). Table 2-2 lists, for each event associated with each gene, a p-value associated with each breast cancer molecular subtype. Each p-value was calculated from the Fisher's exact test with false discovery rate (FDR) correction comparing the number of subjects having a particular breast cancer molecular subtype and the particular event versus the number of subjects having all other breast cancer molecular subtypes and the particular event.

TABLE 2-1

| | | Percentage of Subjects | | | | |
|---|---|---|---|---|---|---|
| Gene | Event | Basal | HER2-high | HER2-low | Luminal A | Luminal B |
| TP53 | M | 82.8851 | 64.6884 | 71.875 | 8.0868 | 19.9101 |
| | DD | 0 | 0.5935 | 0 | 0 | 0.3211 |
| BRCA1 | G | 10.0592 | 0 | 0 | 1.7544 | 0.7533 |
| | M | 5.6235 | 2.0772 | 2.0833 | 1.3807 | 1.413 |
| | DD | 0.2445 | 0 | 0 | 0 | 0 |
| PIK3CA | M | 6.846 | 29.9703 | 53.125 | 60.1578 | 40.334 |
| | AA | 8.802 | 3.5608 | 6.25 | 0.5917 | 0.9634 |
| ERBB2 | M | 1.467 | 3.2641 | 8.3333 | 3.7475 | 2.5048 |
| | AA | 3.1785 | 81.008902 | 5.2083 | 0.9862 | 3.7251 |
| AKT1 | M | 0.2445 | 0.5935 | 7.2917 | 2.3669 | 5.2023 |
| | AA | 0.7335 | 2.0772 | 9.375 | 0.5917 | 0.7065 |
| PTEN | M | 5.1345 | 0.8902 | 11.4583 | 3.7475 | 5.0739 |
| | DD | 6.1125 | 0.2967 | 5.2083 | 1.5779 | 1.6057 |
| CDH1 | M | 0.489 | 3.8576 | 8.3333 | 18.146 | 10.9827 |
| | DD | 0.7335 | 0.5935 | 2.0833 | 1.1834 | 0.6423 |
| GATA3 | M | 0 | 5.0445 | 2.0833 | 12.2288 | 17.0199 |
| | DD | 0 | 0 | 0 | 0 | 0.1927 |
| | AA | 21.7604 | 5.638 | 9.375 | 0.1972 | 0.7707 |
| RB1 | M | 6.1125 | 2.6706 | 4.1667 | 0.3945 | 2.1195 |
| | DD | 2.445 | 0.8902 | 2.0833 | 0.1972 | 0.2569 |
| ARID1A | M | 2.445 | 5.3412 | 2.0833 | 2.5641 | 5.5877 |
| | DD | 0 | 0.5935 | 0 | 0.3945 | 0 |
| MAP3K1 | M | 3.1785 | 3.8576 | 3.125 | 16.9625 | 10.79 |
| | DD | 0.978 | 0 | 0 | 0 | 0.1285 |
| FAT3 | M | 3.423 | 0.8902 | 1.0417 | 0 | 1.3487 |
| | DD | 0 | 0 | 0 | 0 | 0.0642 |
| KMT2C | M | 7.5795 | 8.0119 | 13.5417 | 12.6233 | 11.8818 |
| | DD | 1.2225 | 0 | 0 | 0 | 0.1285 |
| KMT2D | M | 1.7115 | 0.8902 | 5.2083 | 0.5917 | 0.7707 |
| FBXW7 | M | 3.1785 | 1.7804 | 1.0417 | 0.1972 | 0.9634 |
| | DD | 0.489 | 0 | 0 | 0 | 0 |
| CREBBP | M | 2.6895 | 0 | 0 | 0.1972 | 0.578 |
| | DD | 0.978 | 0 | 0 | 0 | 0 |
| MAP2K4 | M | 0.2445 | 1.4837 | 2.0833 | 5.3254 | 4.3674 |
| | DD | 0.489 | 4.1543 | 1.0417 | 1.1834 | 2.2479 |
| JAK1 | M | 0 | 0.2967 | 1.0417 | 1.5779 | 1.413 |
| | DD | 1.2225 | 0.5935 | 0 | 0 | 0 |
| | AA | 3.423 | 2.6706 | 5.2083 | 0.1972 | 0.3854 |
| SF3B1 | M | 0.489 | 1.1869 | 0 | 2.3669 | 3.5967 |
| | AA | 2.445 | 0.5935 | 2.0833 | 0 | 0.6423 |
| CCNE2 | M | 0.2445 | 0 | 0 | 0 | 0.1285 |
| | AA | 23.4719 | 27.5964 | 15.625 | 7.1006 | 22.2222 |
| FGFR1 | M | 0.489 | 0 | 0 | 0.1972 | 0.2569 |
| | AA | 9.5355 | 10.6825 | 4.1667 | 8.8757 | 16.4419 |
| TOP2A | M | 0 | 0.5935 | 0 | 0 | 0.0642 |
| | AA | 1.2225 | 23.4421 | 0 | 1.3807 | 2.2479 |
| EGFR | M | 1.467 | 1.1869 | 3.125 | 0.1972 | 1.5414 |
| | AA | 4.6455 | 3.5608 | 11.4583 | 0.789 | 1.2203 |
| FGFR2 | M | 0.2445 | 0.2967 | 1.0417 | 0 | 0.5138 |
| | AA | 2.6895 | 1.1869 | 4.1667 | 0.3945 | 0.9634 |
| MET | M | 0 | 0.5935 | 0 | 0 | 0.5138 |
| | AA | 3.1785 | 0.5935 | 1.0417 | 0.3945 | 0.7065 |

TABLE 2-2

| | | p-value (α = 0.00001; −log10(α) = 5) | | | | |
|---|---|---|---|---|---|---|
| Gene | Event | Basal | HER2-high | HER2-low | Luminal A | Luminal B |
| TP53 | M | 6.00E−109 | 2.53E−34 | 1.15E−13 | 1.00E+00 | 1.00E+00 |
| | DD | 1.00E+00 | 1.00E+00 | 1.00E+00 | 1.00E+00 | 1.00E+00 |
| BRCA1 | G | 4.96E−09 | 1.00E+00 | 1.00E+00 | 1.00E+00 | 1.00E+00 |
| | M | 4.58E−05 | 1.00E+00 | 1.00E+00 | 1.00E+00 | 1.00E+00 |
| | DD | 1.00E+00 | 1.00E+00 | 1.00E+00 | 1.00E+00 | 1.00E+00 |
| PIK3CA | M | 1.00E+00 | 1.00E+00 | 1.91E−02 | 3.66E−27 | 8.51E−02 |
| | AA | 1.87E−12 | 1.00E+00 | 3.00E−01 | 1.00E+00 | 1.00E+00 |
| ERBB2 | M | 1.00E+00 | 1.00E+00 | 5.42E−02 | 1.00E+00 | 1.00E+00 |
| | AA | 1.00E+00 | 1.02E−230 | 1.00E+00 | 1.00E+00 | 1.00E+00 |
| AKT1 | M | 1.00E+00 | 1.00E+00 | 5.16E−01 | 1.00E+00 | 7.04E−07 |
| | AA | 1.00E+00 | 7.94E−01 | 6.00E−06 | 1.00E+00 | 1.00E+00 |
| PTEN | M | 1.00E+00 | 1.00E+00 | 3.82E−02 | 1.00E+00 | 9.84E−01 |
| | DD | 5.00E−06 | 1.00E+00 | 5.83E−01 | 1.00E+00 | 1.00E+00 |
| CDH1 | M | 1.00E+00 | 1.00E+00 | 1.00E+00 | 1.03E−09 | 1.53E−01 |
| | DD | 1.00E+00 | 1.00E+00 | 1.00E+00 | 1.00E+00 | 1.00E+00 |
| GATA3 | M | 1.00E+00 | 1.00E+00 | 1.00E+00 | 1.00E+00 | 7.41E−20 |
| | DD | 1.00E+00 | 1.00E+00 | 1.00E+00 | 1.00E+00 | 1.00E+00 |
| | AA | 2.59E−47 | 1.00E+00 | 3.90E−01 | 1.00E+00 | 1.00E+00 |
| RB1 | M | 8.60E−05 | 1.00E+00 | 1.00E+00 | 1.00E+00 | 1.00E+00 |
| | DD | 1.35E−03 | 1.00E+00 | 1.00E+00 | 1.00E+00 | 1.00E+00 |
| ARID1A | M | 1.00E+00 | 1.00E+00 | 1.00E+00 | 1.00E+00 | 1.09E−02 |
| | DD | 1.00E+00 | 6.86E−01 | 1.00E+00 | 1.00E+00 | 1.00E+00 |
| MAP3K1 | M | 1.00E+00 | 1.00E+00 | 1.00E+00 | 1.16E−07 | 2.29E−01 |
| | DD | 4.59E−02 | 1.00E+00 | 1.00E+00 | 1.00E+00 | 1.00E+00 |
| FAT3 | M | 5.01E−03 | 1.00E+00 | 1.00E+00 | 1.00E+00 | 1.00E+00 |
| | DD | 1.00E+00 | 1.00E+00 | 1.00E+00 | 1.00E+00 | 1.00E+00 |
| KMT2C | M | 1.00E+00 | 1.00E+00 | 1.00E+00 | 1.00E+00 | 6.03E−01 |
| | DD | 8.88E−03 | 1.00E+00 | 1.00E+00 | 1.00E+00 | 1.00E+00 |
| KMT2D | M | 5.89E−01 | 1.00E+00 | 1.31E−02 | 1.00E+00 | 1.00E+00 |
| FBXW7 | M | 7.45E−03 | 1.00E+00 | 1.00E+00 | 1.00E+00 | 1.00E+00 |
| | DD | 1.98E−01 | 1.00E+00 | 1.00E+00 | 1.00E+00 | 1.00E+00 |
| CREBBP | M | 3.52E−04 | 1.00E+00 | 1.00E+00 | 1.00E+00 | 1.00E+00 |
| | DD | 3.88E−03 | 1.00E+00 | 1.00E+00 | 1.00E+00 | 1.00E+00 |
| MAP2K4 | M | 1.00E+00 | 1.00E+00 | 1.00E+00 | 1.51E−01 | 6.23E−02 |
| | DD | 1.00E+00 | 5.17E−02 | 1.00E+00 | 1.00E+00 | 1.00E+00 |
| JAK1 | M | 1.00E+00 | 1.00E+00 | 1.00E+00 | 1.00E+00 | 8.84E−01 |
| | DD | 1.33E−02 | 1.00E+00 | 1.00E+00 | 1.00E+00 | 1.00E+00 |
| | AA | 1.98E−03 | 2.32E−01 | 7.86E−02 | 1.00E+00 | 1.00E+00 |
| SF3B1 | M | 1.00E+00 | 1.00E+00 | 1.00E+00 | 1.00E+00 | 6.18E−04 |
| | AA | 8.46E−03 | 1.00E+00 | 1.00E+00 | 1.00E+00 | 1.00E+00 |
| CCNE2 | M | 1.00E+00 | 1.00E+00 | 1.00E+00 | 1.00E+00 | 1.00E+00 |
| | AA | 4.32E−01 | 2.95E−03 | 1.00E+00 | 1.00E+00 | 1.70E−02 |
| FGFR1 | M | 1.00E+00 | 1.00E+00 | 1.00E+00 | 1.00E+00 | 1.00E+00 |
| | AA | 1.00E+00 | 1.00E+00 | 1.00E+00 | 1.00E+00 | 3.52E−08 |
| TOP2A | M | 1.00E+00 | 3.71E−01 | 1.00E+00 | 1.00E+00 | 1.00E+00 |
| | AA | 1.00E+00 | 1.97E−44 | 1.00E+00 | 1.00E+00 | 1.00E+00 |
| EGFR | M | 1.00E+00 | 1.00E+00 | 1.00E+00 | 1.00E+00 | 1.00E+00 |
| | AA | 1.04E−02 | 6.66E−01 | 5.90E−05 | 1.00E+00 | 1.00E+00 |
| FGFR2 | M | 1.00E+00 | 1.00E+00 | 1.00E+00 | 1.00E+00 | 1.00E+00 |
| | AA | 8.22E−02 | 1.00E+00 | 2.93E−01 | 1.00E+00 | 1.00E+00 |
| MET | M | 1.00E+00 | 1.00E+00 | 1.00E+00 | 1.00E+00 | 8.43E−01 |
| | AA | 5.60E−04 | 1.00E+00 | 1.00E+00 | 1.00E+00 | 1.00E+00 |

### Basal Molecular Subtype

In some embodiments, the Basal molecular subtype is similar to the TNBC immunohistochemistry (IHC) phenotype (which is 82.3% of the Basal molecular subtype).

In some embodiments, the Basal molecular subtype is characterized by the lowest 5-year survival rate (along with the HER2-low molecular subtype) relative to the other breast cancer molecular subtypes. For example, in the SCAN B cohort, subjects having the Basal and HER2-low molecular subtypes had a 77% 5-year survival rate, which was the lowest relative to the other breast cancer molecular subtypes. In some embodiments, the Basal molecular subtype is characterized by a lower 5-year survival rate compared to the Luminal A and Luminal B molecular subtypes, but a higher 5-year survival rate compared the HER2-high and HER2-low molecular subtypes. For example, in the Metabric cohort, subjects having the Basal molecular subtype had a 66% 5-year survival rate, which was lower than Luminal A and Luminal B molecular subtypes but higher than HER2-high and HER2-low molecular subtypes.

In some embodiments, the coding mutation rate of TP53 in the Basal molecular subtype is the highest among the other breast cancer molecular subtypes. In some embodiments, the BRCA1 germline mutation is a relatively common event for Basal molecular subtype samples along with BRCA1 somatic mutation, relative to the other breast cancer molecular subtypes. In some embodiments, compared to the other breast cancer molecular subtypes, amplifications in GATA3 and PIK3CA occur more often in the Basal molecular subtype.

In some embodiments, PIK3CA of GATA3 mutations occur less in the Basal molecular subtype than in the Luminal molecular subtypes. In some embodiments, compared to the Luminal molecular subtypes, deep deletions of PTEN are more characteristic of the Basal molecular subtype.

In some embodiments, the Basal molecular subtype is characterized by high expression of basal phenotype marker genes (e.g., FOXC1 and KRT11) relative to the other breast cancer molecular subtypes. In some embodiments, the Basal molecular subtype is characterized by low expression of luminal marker genes (e.g., FOXA1 and GATA3) relative to the other breast cancer molecular subtypes. In some embodiments, the Basal molecular subtype is characterized by low expression of hormone receptor genes (e.g., AR, ESR1, and PGR) relative to the other breast cancer molecular subtypes. In some embodiments, the Basal molecular subtype is characterized by high expression of cell cycle (e.g., MYC, MKI67, CCNE1, CDK1, CCNB1, MYBL2, CCNB2, E2F2, and CDC20) gene signatures relative to the other breast cancer molecular subtypes. In some embodiments, the Basal molecular subtype is characterized by high expression of mitosis (e.g., PTTG1, AURKB, NDC80, KIF2C, TPX2, and TOP2A) gene signatures relative to the other breast cancer molecular subtypes. In some embodiments, the Basal molecular subtype is characterized by lowest expression of kinase receptor genes (e.g., ERBB2 and ERBB3) relative to the other breast cancer molecular subtypes. In some embodiments, the Basal molecular subtype is characterized by an expression of EGFR that is second highest among the breast cancer molecular subtypes (after the HER2-low molecular subtype). In some embodiments, the Basal molecular subtype is characterized by highest expression of differentiation marker gene KRT17 relative to the other breast cancer molecular subtype. In some embodiments, the Basal molecular subtype is characterized by highest expression of genes CDH3, UBE2C, UBE2T, TOP2A, BIRC5, PHGDH and TYMS relative to the other breast cancer molecular subtypes. In some embodiments, the Basal molecular subtype is characterized by lowest expression of AGR2, XBP1, TFF1 and CA12 relative to the other breast cancer molecular subtypes. In some embodiments, the Basal molecular subtype is the leading molecular subtype in terms of JAK-STAT, MAPK, NFkB, PI3K and TNFa signaling pathways, while p53 and androgen pathways have the lowest signaling in the Basal molecular subtype.

In some embodiments, the Basal molecular subtype is characterized by an increased number of NK-cells relative to the HER2-high, Luminal A, and Luminal B molecular subtypes. In some embodiments, the Basal molecular subtype is characterized by an increase number of B-cells relative to Luminal A and Luminal B molecular subtypes. In some embodiments, the Basal molecular subtype is characterized by an increased number of CD4+ T-cells relative to the Luminal A and Luminal B molecular subtypes. In some embodiments, the Basal molecular subtype is characterized by a reduced number of fibroblasts relative to the HER2-high, Luminal A and Luminal B molecular subtypes.

HER2-high Molecular Subtype

In some embodiments, the HER2-high molecular subtype demonstrates common expression, mutational, and amplification features specific to its subtype. In some embodiments, the HER2-high molecular subtype is characterized by HER2 positivity by immunohistochemistry (IHC). For example, 91% of samples having the HER2-high molecular subtype showed HER2 positivity by IHC according to TCGA, Metabrick, SCAN-B annotation. In some embodiments, the HER2-high molecular subtype is characterized by a 5-year survival rate that is lower than the 5-year survival rate of subjects having the Basal, Luminal A, and Luminal B molecular subtypes. For example, the 5-years survival rate for subjects having the HER2-high molecular subtype in the Metabric dataset was 60%, which was lower than in the Basal, Luminal A and Luminal B molecular subtypes. In some embodiments, the HER2-high molecular subtype is characterized by a 5-year survival rate that is higher than the 5-year survival rate of subjects having the Basal and HER2-low molecular subtypes. For example, in the SCAN-B dataset the 5-year HER2-high molecular subtype survival was 86%, which was higher than the Basal and HER2-low molecular subtypes.

In some embodiments, the HER2-high molecular subtype is characterized by a genomic profile showing similar mutational events as reported by Brueffer, Christian, et al. (“The mutational landscape of the SCAN-B real-world primary breast cancer transcriptome.” EMBO molecular medicine 12.10 (2020): e12118). In some embodiments, the HER2-high molecular subtype is characterized by amplifications in ERBB2 relative to other breast cancer molecular subtypes. In some embodiments, the HER2-high molecular subtype is characterized by a significantly high percentage of coding mutations in the TP53 gene compared to the HER2-low, Luminal A, and Luminal B molecular subtypes. In some embodiments, the HER2-high molecular subtype is characterized by the highest expression of ERBB2 relative to the Basal, HER2-low and Luminal molecular subtypes. In some embodiments, the HER2-high molecular subtype is characterized by an expression of the ERBB3 gene that is higher than in the HER2-low molecular subtype. In some embodiments, the HER2-high molecular subtype is characterized by high expression of cell cycle (e.g., MKI67, CDK1, CCNB1) genes relative to the HER2-low, Luminal A, and Luminal B molecular subtypes. In some embodiments, the HER2-high molecular subtype is characterized by high expression of mitosis genes (e.g., TPX2, AURKB, NDC80) relative to the HER2-low, Luminal A, and Luminal B molecular subtypes. In some embodiments, the HER2-high molecular subtype is characterized by high expression of markers UBE2C, UBE2T, and TOP2A relative to the HER2-low, Luminal A, and Luminal B molecular subtypes. In some embodiments, the HER2-high molecular subtype is characterized by a bimodal distribution of the ESR1 gene, which may be explained by heterogeneity of hormone status by IHC in this subtype.

In some embodiments, the HER2-high molecular subtype is characterized by an EGFR pathway having a statistically significant high score relative to the other breast cancer molecular subtypes. In some embodiments, the HER2-high molecular subtype is characterized by a deconvolution profile having a low level of Naive T-cells and Naive T-CD8 T-cells compared to the Basal, Luminal A, and Luminal B molecular subtypes.

### HER2-low Molecular Subtype

In some embodiments, the HER2-low molecular subtype is characterized by a low rate of HER2-positivity by IHC relative to the HER2-high molecular subtype. In some embodiments, the HER2-low molecular subtype is characterized by LAR class enrichment when compared to the Burstein classification of triple-negative breast cancer (TNBC) samples. In some embodiments, the HER2-low molecular subtype is characterized by a 5-year survival rate that is similar to the 5-year survival rate of subjects having the HER2-high molecular subtype and worse than the 5-year survival rate of subjects having the Basal, Luminal A, and Luminal B molecular subtypes. For example, 5-year survival of subjects having the HER2-low molecular subtype in the Metabric dataset was similar to the 5-year survival of subjects having the HER2-high molecular subtype and was the worst among all breast cancer molecular subtypes. In some embodiments, the HER2-low molecular subtype is characterized by a 5-year survival rate that is similar to the 5-year survival rate of subjects having the Basal molecular subtype and worse than the 5-year survival rate of subjects having HER2-low, Luminal A, and Luminal B molecular subtypes. For example, the 5-years survival rate of subjects having the HER2-low molecular subtype in the SCAN-B dataset was similar to the 5-year survival rate of subjects having the Basal subtype and worst among all breast cancer molecular subtypes. In some embodiments, the HER2-low molecular subtype is characterized by a 10-year survival rate that is similar to the 10-year survival rate of subjects having the HER2-high molecular subtype. For example, 10-year survival of subjects having the HER2-low molecular subtype in Metabric is similar to the 10-year survival of subject having the HER2-high molecular subtype. In some embodiments, the HER2-low molecular subtype is characterized by a 15-year survival rate that is worse than all other breast cancer molecular subtypes.

In some embodiments, the HER2-low molecular subtype is characterized by a low rate of HER2-amplifications measured by the FISH method relative to the HER2-high molecular subtype. In some embodiments, the HER2-low molecular subtype is characterized by a low rate of amplifications) relative to the HER2-high molecular subtype.

In some embodiments, the HER2-low molecular subtype with respect to the IHC classification is characterized by 1+ or 2+ scores without amplifications (e.g., by the FISH method). In some embodiments, the HER2-low molecular subtype is characterized by an ERBB2 expression between 6 and 8.5 log 2.

In some embodiments, the HER2-low molecular subtype is characterized by a high rate of amplification of the EGFR gene relative to the other breast cancer molecular subtypes. For example, investigation of CNA statistics showed that the HER2-low molecular subtype had the highest percentage of samples with amplifications of the EGFR gene among the other breast cancer molecular subtypes. In some embodiments, the HER2-low molecular subtype is characterized by a high percentage of amplifications in AKT1 relative to the other breast cancer molecular subtypes. In some embodiments, the HER2-low molecular subtype is characterized by a high percentage of coding mutations in TP53) relative to all other breast cancer molecular subtypes. In some embodiments, the HER2-low molecular subtype is characterized by a high percentage of coding mutations in PIK3CA relative to all other breast cancer molecular subtypes. In some embodiments, the HER2-low molecular subtype is characterized by a high percentage of coding mutations in ERBB2 relative to all other breast cancer molecular subtypes. In some embodiments, the HER2-low molecular subtype is characterized by a high percentage of coding mutations in KMT2D relative to all other breast cancer molecular subtypes. In some embodiments, the HER2-low molecular subtype is characterized by a high percentage of coding mutations in PTEN relative to all other breast cancer molecular subtypes. In some embodiments, the HER2-low molecular subtype is characterized by the presence of missense mutations in the tyrosine kinase domain of the ERBB2 gene. In some embodiments, the HER2-low molecular subtype is characterized by the presence of missense and frame shift mutations in the TP53 gene. In some embodiments, the HER2-low molecular subtype is characterized by the presence of missense and multi-hit mutations in the PIK3CA gene.

In some embodiments, the HER2-low molecular subtype is characterized by the highest expression of EGFR and CLDN8 relative to all other breast cancer molecular subtypes. In some embodiments, the HER2-low molecular subtype is characterized by the lowest expression of IGFR1 relative to all other breast cancer molecular subtypes. In some embodiments, the HER2-low molecular subtype is characterized by the lowest expression of cell migration marker AGR2 relative to all other breast cancer molecular subtypes. In some embodiments, the HER2-low molecular subtype is characterized by the lowest expression of luminal markers GATA3 and FOXA1 relative to all other breast cancer molecular subtypes. In some embodiments, the HER2-low molecular subtype is characterized by the lowest expression of hormone receptors ESR1 and PGR relative to all other breast cancer molecular subtypes. In some embodiments, the HER2-low molecular subtype is characterized by the lowest expression of tyrosine kinase receptor ERBB3 relative to all other breast cancer molecular subtypes. In some embodiments, the HER2-low molecular subtype is characterized by the lowest expression of markers CA12, XBP1, and TFF1 relative to all other breast cancer molecular subtypes. In some embodiments, the HER2-low molecular subtype is characterized by the highest level of Androgen, Hypoxia, and p53, NFkB, TNFa pathways relative to all other breast cancer molecular subtypes. In some embodiments, the HER2-low molecular subtype is characterized by the lowest level of Estrogen pathway score relative to all other breast cancer molecular subtypes.

In some embodiments, the HER2-low molecular subtype is characterized by a significantly higher level of regulatory NK-cells relative to the Basal, Luminal A, and Luminal B molecular subtypes. In some embodiments, the HER2-low molecular subtype is characterized by a lower level of Naive T-cells and Naive CD8 T-cells relative to the Basal, Luminal A, and Luminal B molecular subtypes.

In some embodiments, the HER2-low molecular subtype is characterized by its distinctive relationship with cell cycle dynamics, underscored by differential gene expression that encompasses key processes such as Differentiation, Kinase Receptor Signaling, Cell Cycle Regulation, and Luminal Phenotype Characteristics. In some embodiments, the HER2-low molecular subtype is characterized by overexpression of several genes crucial for cell cycle progression, such as MYBL2, RRM2, CDC20, MKI67, and BIRC5, particularly when juxtaposed against Luminal A and Luminal B molecular subtype tumors. This upregulation hints at an enhanced cell proliferation potential, a feature further emphasized by CCNE1's overexpression, which implies an accelerated G1 to S phase transition. In contrast, in some embodiments, this expression may be notably subdued when compared to Basal subtypes, suggesting distinct proliferation dynamics across these breast cancer molecular subtypes. Additionally, in some embodiments, the fluctuation of mitotic genes like MELK and TOP2A might impact mitotic integrity and dynamics, with BIRC5 (Survivin) overexpression indicating potential resistance to apoptosis-inducing therapies in the HER2-low molecular subtype context.

In some embodiments, the HER2-low molecular subtype straddles the line between basal and luminal phenotypes, exhibiting a complex basal-luminal identity. In some embodiments, the HER2-low molecular subtype is characterized by overexpression of KRT5, which points to basal features, contrasting with its reduced expression compared to Basal molecular subtypes. In some embodiments, the HER2-low molecular subtype is characterized by overexpression of FOXA1, which suggests luminal characteristics. The mixed basal-luminal identity may be further complicated by the expression patterns of hormonal signaling genes. In some embodiments, the HER2-low molecular subtype is characterized by lower levels of hormone-related genes such as ESR1, PGR, and GATA3 compared to the other breast cancer molecular subtypes, especially in comparison to Luminal molecular subtype tumors, indicating reduced estrogen and progesterone signaling. However, in some embodiments, the expression of these markers increases when HER2-low is compared to Basal tumors, including AR, hinting at a potential responsiveness to hormonal interventions.

In some embodiments, HER2-low molecular subtype tumors are characterized by the overexpression of EGFR compared to HER2-high and Luminal molecular subtype tumors. This may indicate a reliance on alternative growth and survival pathways, potentially opening avenues for targeted EGFR therapies. In some embodiments, the HER2-low molecular subtype is characterized by an increased ERBB2 expression compared to the Basal molecular subtype, Despite its nomenclature, underlining the complexity of its molecular landscape.

In some embodiments, the HER2-low molecular subtype is characterized by overexpression of genes involved in cell adhesion and migration, such as CLDN8, CDH3, SFRP1, MMP11, and TMEM45B, relative to the other breast cancer molecular subtypes. This overexpression may suggest alterations in cell-cell adhesion and potential changes in migratory or invasive properties, impacting tumor aggressiveness and metastatic capability.

In some embodiments, the metabolic landscape of HER2-low molecular subtype tumors is characterized by the expression of genes like PHGDH, SLC39A6, NAT1, and CA12, which are downregulated compared to the HER2-high molecular subtype. This pattern reflects a nuanced interplay of amino acid synthesis, ion homeostasis, detoxification processes, and pH regulation, suggesting potential metabolic vulnerabilities within these tumors. Notably, the expression of these genes varies when compared to Luminal and Basal molecular subtypes, underscoring the metabolic heterogeneity within HER2-low molecular subtype tumors.

In some embodiments, the Luminal A and Luminal B molecular subtypes are associated with the HR+/HER2−IHC phenotype. For example, Luminal samples almost entirely consisted of samples with HR+/HER2− IHC phenotype.

In some embodiments, the Luminal molecular subtypes are characterized by high expression of hormone receptor genes ESR1 and PGR relative to all other breast cancer molecular subtypes. In some embodiments, the Luminal molecular subtypes are characterized by high expression of luminal marker genes GATA3 and FOXA1 relative to all other breast cancer molecular subtypes. In some embodiments, the Luminal molecular subtypes are characterized by high expression of kinase receptor genes ERBB3 and IGF1R relative to all other breast cancer molecular subtypes. In some embodiments, the Luminal molecular subtypes are characterized by high expression of cell migration gene AGR3 along with low expression of CDH3 relative to all other breast cancer molecular subtypes. In some embodiments, the Luminal molecular subtypes are characterized by low expression of basal marker gene SOX11 relative to all other breast cancer molecular subtypes. In some embodiments, the Luminal molecular subtypes are characterized by low expression of cell cycle genes CCNE1, MYBL2, CCNB2, CDC20 and RRM2 relative to all other breast cancer molecular subtypes. In some embodiments, the Luminal molecular subtypes are characterized by high expression of genes TFF1, CA12, XBP1, MDM2 and low expression of gene PHGDH relative to all other breast cancer molecular subtypes. In some embodiments, in terms of signaling, estrogen pathway is more active in the Luminal molecular subtypes while EGFR, Hypoxia, MAPK, NFkB, PI3K and TNFa pathways are less active than in other subtypes.

### Luminal A Molecular Subtype

In some embodiments, the Luminal A molecular subtype is characterized as the least aggressive subtype of the breast cancer molecular subtypes with the most favorable prognosis. Five-year survival of Luminal A samples was the best in all three datasets: 93% in Metabric, 88% in TCGA and 93% in SCANB. In some embodiments, the Luminal A molecular subtype is characterized by mutations in genes PIK3CA, CDH1 and MAP3K1. In particular, mutations in genes PIK3CA, CDH!, and MAP3K1 had high statistical significance by right tailed Fisher's exact test comparing Luminal A vs all.

In some embodiments, the Luminal A molecular subtype is characterized by a lower expression of cell cycle genes MKI67, CDK1, CCNB1, CCNE1, MYBL2, CCNB2, CDC20, RRM2 and E2F2 relative to the Luminal B molecular subtype. In some embodiments, the Luminal A molecular subtype is characterized by a lower expression of mitosis genes KIF2C, PTTG1, TPX2, NDC80, AURKB relative to the Lower B molecular subtype. In some embodiments, the Luminal A molecular subtype is characterized by the highest expression of the hormone receptor gene PGR relative to all other breast cancer molecular subtypes. In some embodiments, the Luminal A molecular subtype is characterized by the second highest expression of basal marker gene FOXC1 (after Basal) relative to all other breast cancer molecular subtypes. In some embodiments, the Luminal A molecular subtype is characterized by the second highest expression of cell cycle gene MYC (after Basal) relative to all other breast cancer molecular subtypes. In some embodiments, the Luminal A molecular subtype is characterized by the second highest expression of differentiation marker gene KRT17 (after Basal) relative to all other breast cancer molecular subtypes. In some embodiments, the Luminal A molecular subtype is characterized by the lowest expression of UBE2T, UBE2C, TOP2A, BIRC5 and TYMS genes relative to all other breast cancer molecular subtypes.

In some embodiments, the Luminal A molecular subtype is characterized as having the most active TGFb and WNT signaling and the least active PI3K signaling relative to all other breast cancer molecular subtypes.

All the above-mentioned genes and pathways were significantly different in terms of expression and signaling for Luminal A comparing to each of other subtypes.

In some embodiments, the Luminal A molecular subtype is characterized by an increased number of fibroblasts) and endothelium) relative to Basal, HER2-low, and Luminal B molecular subtypes. In some embodiments, the Luminal molecular subtype is characterized by a decreased number of macrophages relative to all other breast cancer molecular subtypes.

### Luminal B Molecular Subtype

In some embodiments, the Luminal B molecular subtype has many common features with the Luminal A molecular subtype. However, in some embodiments, the Luminal B molecular subtype is more proliferative and aggressive. In some embodiments, five-year survival of subjects having the Luminal B molecular subtype is worse than subjects having the Luminal A subtype, but better than subjects having other breast cancer molecular subtypes.

In some embodiments, the Luminal B molecular subtype is characterized by mutations in AKT1 and GATA3.

In some embodiments, the Luminal B molecular subtype is characterized by the highest expression of luminal marker genes FOXA1 and GATA3 and hormone receptor gene ESR1 relative to all other breast cancer molecular subtypes. In some embodiments, the Luminal B molecular subtype is characterized by the highest expression of kinase receptor ERBB3 relative to all other breast cancer molecular subtypes. In some embodiments, the Luminal B molecular subtype is characterized by the lowest expression of EGFR relative to all other breast cancer molecular subtypes. In some embodiments, the Luminal B molecular subtype is characterized by the lowest expression of basal marker FOXC1 and differentiation marker KRT17 relative to all other breast cancer molecular subtypes. In terms of cell migration genes, in some embodiments, the Luminal B molecular subtype is the leader in expression of AGR2 and AGR3, while CDH3 and CLDN8 are the least expressed in the Luminal B molecular subtype, relative to all other breast cancer molecular subtypes. In some embodiments, the Luminal B molecular subtype is characterized by the highest expression of genes XBP1, TFF1, CA12 and MDM2 relative to all other breast cancer molecular subtypes.

In some embodiments, the Luminal B molecular subtype is characterized by the most active estrogen signaling and least active hypoxia, MAPK, NFkB, TNFa and trail signaling relative to all other breast cancer molecular subtypes.

In some embodiments, the Luminal B molecular subtype is characterized by a decreased number of Secreting B cells relative to all other breast cancer molecular subtypes.

### Biological Samples

Aspects of the disclosure relate to methods for determining a breast cancer molecular subtype of a subject by obtaining sequencing data from a biological sample that has been obtained from the subject.

The biological sample may be from any source in the subject's body including, but not limited to, any fluid such as blood (e.g., whole blood, blood serum, or blood plasma), lymph node, breast, etc. Other source in the subject's body may be from saliva, tears, synovial fluid, cerebrospinal fluid, pleural fluid, pericardial fluid, ascitic fluid, and/or urine, hair, skin (including portions of the epidermis, dermis, and/or hypodermis), oropharynx, laryngopharynx, esophagus, bronchus, salivary gland, tongue, oral cavity, nasal cavity, vaginal cavity, anal cavity, bone, bone marrow, brain, thymus, spleen, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

The biological sample may be any type of sample including, for example, a sample of a bodily fluid, one or more cells, one or more pieces of tissue(s) or organ(s). In some embodiments, the biological sample comprises breast tissue sample of the subject. In some embodiments, a breast tissue sample comprises one or more cell types derived from a breast (e.g., epithelial cells, secretory luminal cells, basal/myoepithelial cells, etc.). In some embodiments, a breast tissue sample comprises tumor cells.

In some embodiments, a tissue sample may be obtained from a subject using a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

A sample of lymph node or blood, in some embodiments, refers to a sample comprising cells, e.g., cells from a blood sample or lymph node sample. In some embodiments, the sample comprises non-cancerous cells. In some embodiments, the sample comprises pre-cancerous cells. In some embodiments, the sample comprises cancerous cells. In some embodiments, the sample comprises blood cells. In some embodiments, the sample comprises lymph node cells. In some embodiments, the sample comprises lymph node cells and blood cells.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

In some embodiments, a sample of blood is collected to obtain the cell-free nucleic acid (e.g., cell-free DNA) in the blood.

In some embodiments, the sample may be from a cancerous tissue or an organ or a tissue or organ suspected of having one or more cancerous cells. In some embodiments, the sample may be from a healthy (e.g., non-cancerous) tissue or organ. In some embodiments, a sample from a subject (e.g., a biopsy from a subject) may include both healthy and cancerous cells and/or tissue. In certain embodiments, one sample will be taken from a subject for analysis. In some embodiments, more than one (e.g., 2, 3, 4, 5, 6, 7, 8, 9, 10, 11, 12, 13, 14, 15, 16, 17, 18, 19, 20, or more) samples may be taken from a subject for analysis. In some embodiments, one sample from a subject will be analyzed. In certain embodiments, more than one (e.g., 2, 3, 4, 5, 6, 7, 8, 9, 10, 11, 12, 13, 14, 15, 16, 17, 18, 19, 20, or more) samples may be analyzed. If more than one sample from a subject is analyzed, the samples may be procured at the same time (e.g., more than one sample may be taken in the same procedure), or the samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same region (e.g., from the same tumor or area of tissue) or a different region (including, e.g., a different tumor). A second or subsequent sample may be taken or obtained from the subject after one or more treatments, and may be taken from the same region or a different region. As a non-limiting example, the second or subsequent sample may be useful in determining whether the cancer in each sample has different characteristics (e.g., in the case of samples taken from two physically separate tumors in a patient) or whether the cancer has responded to one or more treatments (e.g., in the case of two or more samples from the same tumor prior to and subsequent to a treatment).

Any of the biological samples described herein may be obtained from the subject using any known technique. See, for example, the following publications on collecting, processing, and storing biological samples, each of which is incorporated by reference herein in its entirety: Biospecimens and biorepositories: from afterthought to science by Vaught et al. (Cancer Epidemiol Biomarkers Prev. 2012 February; 21(2):253-5), and Biological sample collection, processing, storage and information management by Vaught and Henderson (IARC Sci Publ. 2011; (163):23-42).

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one form to another form such that the first form is no longer detected at the same level as before degradation.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris·Cl; 0.5 mM EDTA, pH 9.0)) and other coagulants, and Acids Citrate Dextrose (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −8-° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

### Expression Data

Aspects of the disclosure relate to methods of determining a breast cancer molecular subtype for a subject using sequencing data or RNA expression data obtained from a biological sample (e.g., a tumor sample) from the subject.

The RNA expression data used in methods described herein typically is derived from sequencing data obtained from the biological sample.

The sequencing data may be obtained from the biological sample using any suitable sequencing technique and/or apparatus (e.g., sequencing platform 104 shown in FIG. 1A and FIG. 1E). In some embodiments, the sequencing apparatus used to sequence the biological sample may be selected from any suitable sequencing apparatus known in the art including, but not limited to, Illumina™, SOLid™, Ion Torrent™, PacBio™, a nanopore-based sequencing apparatus, a Sanger sequencing apparatus, or a 454™ sequencing apparatus. In some embodiments, sequencing apparatus used to sequence the biological sample is an Illumina sequencing (e.g., NovaSeq™, NextSeq™, HiSeq™, MiSeq™, or MiniSeq™) apparatus. After the sequencing data is obtained, it is processed in order to obtain the RNA expression data. RNA expression data may be acquired using any method known in the art including, but not limited to whole transcriptome sequencing, whole exome sequencing, total RNA sequencing, mRNA sequencing, targeted RNA sequencing, RNA exome capture sequencing, next generation sequencing, and/or deep RNA sequencing. In some embodiments, RNA expression data may be obtained using a microarray assay.

In some embodiments, the sequencing data is processed to produce RNA expression data. In some embodiments, RNA sequence data is processed by one or more bioinformatics methods or software tools, for example RNA sequence quantification tools (e.g., Kallisto) and genome annotation tools (e.g., Gencode v23), in order to produce expression data. The Kallisto software is described in Nicolas L Bray, Harold Pimentel, Pill Melsted and Lior Pachter, Near-optimal probabilistic RNA-seq quantification, Nature Biotechnology 34, 525-527 (2016), doi:10.1038/nbt.3519, which is incorporated by reference in its entirety herein.

In some embodiments, microarray expression data is processed using a bioinformatics R package, such as “affy” or “limma,” in order to produce expression data. The “affy” software is described in Bioinformatics. 2004 Feb. 12; 20(3):307-15. doi: 10.1093/bioinformatics/btg405. “affy—analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie M E, Phipson B, Wu D, Hu Y, Law C W, Shi W, Smyth G K “limma powers differential expression analyses for RNA-sequencing and microarray studies.” Nucleic Acids Res. 2015 Apr. 20; 43(7):e47. 20. doi.org/10.1093/nar/gkv007PMID: 25605792, PMCID: PMC4402510, which is incorporated by reference herein its entirety.

In some embodiments, sequencing data and/or expression data comprises more than 5 kilobases (kb). In some embodiments, the size of the obtained RNA data is at least 10 kb. In some embodiments, the size of the obtained RNA sequencing data is at least 100 kb. In some embodiments, the size of the obtained RNA sequencing data is at least 500 kb. In some embodiments, the size of the obtained RNA sequencing data is at least 1 megabase (Mb). In some embodiments, the size of the obtained RNA sequencing data is at least 10 Mb. In some embodiments, the size of the obtained RNA sequencing data is at least 100 Mb. In some embodiments, the size of the obtained RNA sequencing data is at least 500 Mb. In some embodiments, the size of the obtained RNA sequencing data is at least 1 gigabase (Gb). In some embodiments, the size of the obtained RNA sequencing data is at least 10 Gb. In some embodiments, the size of the obtained RNA sequencing data is at least 100 Gb. In some embodiments, the size of the obtained RNA sequencing data is at least 500 Gb.

In some embodiments, the expression data is acquired through bulk RNA sequencing. Bulk RNA sequencing may include obtaining expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

In some embodiments, the expression data comprises next-generation sequencing (NGS) data. In some embodiments, the expression data comprises microarray data.

Expression data (e.g., indicating expression levels) for a plurality of genes may be used for any of the methods or compositions described herein. The number of genes which may be examined may be up to and inclusive of all the genes of the subject. In some embodiments, expression levels may be determined for all of the genes of a subject. As a non-limiting example, In some embodiments, expression levels may be obtained for at least 25 genes, at least 50 genes, at least 75 genes, at least 100 genes, at least 150 genes, at least 200 genes, at least 250 genes, at least 500 genes, at least 1,000 genes, at least 1,500 genes, at least 2,000 genes, at least 2,500 genes, at least 3,000 genes, at least 3,500 genes, at least 4,000 genes, at least 4,500 genes, at least 5,000 genes, at least 6000 genes, at least 7,000 genes, at least 8,000 genes, at least 9,000 genes, at least 10,000 genes, at least 15,000 genes, at least 20,000 genes, or at least any other suitable number of genes, as aspects of the technology described herein are not limited in this respect. In some embodiments, expression levels may be obtained for at most 25 genes, at most 50 genes, at most 75 genes, at most 100 genes, at most 150 genes, at most 200 genes, at most 250 genes, at most 500 genes, at most 1,000 genes, at most 1,500 genes, at most 2,000 genes, at most 2,500 genes, at most 3,000 genes, at most 3,500 genes, at most 4,000 genes, at most 4,500 genes, at most 5,000 genes, at most 6000 genes, at most 7,000 genes, at most 8,000 genes, at most 9,000 genes, at most 10,000 genes, at most 15,000 genes, at most 20,000 genes, or at most any other suitable number of genes, as aspects of the technology described herein are not limited in this respect. It should be appreciated that any of the above-listed upper bounds may be coupled with any of the above-listed lower bounds. In some embodiments, As another set of non-limiting examples, the expression data may include, for each set of genes listed in Table 1, expression data for at least some (e.g., all) of the genes included in the particular set of genes.

In some embodiments, RNA expression data is obtained by accessing the RNA expression data from at least one computer storage medium on which the RNA expression data is stored. Additionally or alternatively, in some embodiments, RNA expression data may be received from one or more sources via a communication network of any suitable type. For example, in some embodiment, the RNA expression data may be received from a server (e.g., a SFTP server, or Illumina BaseSpace).

The RNA expression data obtained may be in any suitable format, as aspects of the technology described herein are not limited in this respect. For example, in some embodiments, the RNA expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

Expression data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

In some embodiments, sequencing data is processed to obtain RNA expression data from the sequencing data. For example, the sequencing data may be processed using any suitable computing device or devices, as aspects of the technology described herein are not limited in this respect. For example, the processing may be performed by a computing device part of a sequencing apparatus. In other embodiments, the processing may be performed by one or more computing devices external to the sequencing apparatus.

In some embodiments, processing the sequencing data to obtain RNA expression data from the sequencing data includes normalizing the sequencing data to transcripts per kilobase million (TPM) units. The normalization may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM normalization may be performed using a software package, such as, for example, the gcrma package. Aspects of the gcrma package are described in Wu J, Gentry RIwcfJMJ (2021). “gcrma: Background Adjustment Using Sequence Information. R package version 2.66.0.,” which is incorporated by reference in its entirety herein. In some embodiments, RNA expression level in TPM units for a particular gene may be calculated according to the following formula:

$$A \cdot \frac{1}{\sum ( A )} \cdot {10}^{6}$$

$$where$$

$$A = \frac{total reads mapped to gene \cdot {10}^{3}}{gene length in bp}$$

Next, in some embodiments, the RNA expression levels in TPM units may be log transformed.

In some embodiments, the RNA expression levels may not be normalized to transcripts per million units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

In some embodiments, the RNA expression data is obtained by processing sequence data generated by a sequencing protocol (e.g., the series of nucleotides in a nucleic acid molecule identified by next-generation sequencing, sanger sequencing, etc.) as well as information contained therein (e.g., information indicative of source, tissue type, etc.) which may also be considered information that can be inferred or determined from the sequence data. In some embodiments, expression data obtained by processing the sequence data can include information included in a FASTA file, a description and/or quality scores included in a FASTQ file, an aligned position included in a BAM file, and/or any other suitable information obtained from any suitable file.

In some embodiments, enrichment scores for genes in one or more sets of genes are determined. For example, an enrichment score (e.g., enrichment score 154-4 in FIG. 1D) may be determined for at least some genes in the set of genes listed for the Luminal B subtype in Table 1. Additionally, or alternatively, an enrichment score (e.g., enrichment score 154-5 in FIG. 1D) may be determined for at least some genes in the set of genes listed for the Luminal A subtype in Table 1. In some embodiments, an enrichment score is generated using a gene set enrichment analysis (GSEA) technique, using RNA expression levels of at least some genes in a set of genes. In some embodiments, using a GSEA technique comprises using single-sample GSEA.

Aspects of single sample GSEA (ssGSEA) are described in Barbie et al. Nature. 2009 Nov. 5; 462(7269): 108-112, the entire contents of which are incorporated by reference herein. In some embodiments, ssGSEA is performed according to the following formula:

$$ssGSEA score = \frac{{\sum }_{i}^{N} {r}_{i}^{1.25}}{{\sum }_{i}^{N} {r}_{i}^{0.25}} - \frac{( M - N + 1 )}{2}$$

where rᵢ represents the rank of the ith gene in expression matrix, where N represents the number of genes in the gene set, and where M represents total number of genes in expression matrix. Additional, suitable techniques of performing GSEA are known in the art and are contemplated for use in the methods described herein without limitation. In some embodiments, an enrichment score is calculated by performing ssGSEA on expression data from a plurality of subjects, for example expression data from one or more cohorts of subjects, such as TCGA, Metabric, FUSCCTNBC, GSE103091, GSE106977, GSE21653, GSE25066, GSE41998, GSE47994, GSE81538, GSE96058, etc., in order to produce a plurality of enrichment scores.

### Methods of Treatment

In some aspects, a cancer therapy is identified for and/or administered to a subject determined to have a particular breast cancer molecular subtype. For example, trastuzumab deruxtecan may be administered or recommended for administration to a subject when a tumor sample from the subject is determined to have a HER2-low molecular subtype of breast cancer.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor formation, tumor growth, molecular category identified for the cancer using the techniques described herein) may be analyzed.

Generally, for administration of any of the anti-cancer antibodies described herein, an initial candidate dosage may be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner (e.g., a medical doctor) wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays. The dosing regimen (including the therapeutic used) may vary over time.

When the anti-cancer therapeutic agent is not an antibody, it may be administered at the rate of about 0.1 to 300 mg/kg of the weight of the subject divided into one to three doses, or as disclosed herein. In some embodiments, for an adult subject of normal weight, doses ranging from about 0.3 to 5.00 mg/kg may be administered. The particular dosage regimen, e.g., dose, timing, and/or repetition, will depend on the particular subject and that individual's medical history, as well as the properties of the individual agents (such as the half-life of the agent, and other considerations well known in the art).

Dosing of trastuzumab deruxtecan is well-known, for example as described by Rugo, H. S., et al. ESMO open 7.4 (2022): 100553. For example, dosages of trastuzumab deruxtecan include administration of 5.4 mg/kg or 6.4 mg/kg every 3 weeks, by infusion.

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of the cancer, or the predisposition toward a cancer.

Alleviating a cancer includes delaying the development or progression of the disease or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given period and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

In some embodiments, the anti-cancer therapeutic agent described herein is administered to a subject in need of the treatment at an amount sufficient to reduce cancer (e.g., tumor) growth by at least 10% (e.g., 20%, 30%, 40%, 50%, 60%, 70%, 80%, 90% or greater). In some embodiments, the anti-cancer therapeutic agent described herein is administered to a subject in need of the treatment at an amount sufficient to reduce cancer cell number or tumor size by at least 10% (e.g., 20%, 30%, 40%, 50%, 60%, 70%, 80%, 90% or more). In other embodiments, the anti-cancer therapeutic agent is administered in an amount effective in altering cancer type. Alternatively, the anti-cancer therapeutic agent is administered in an amount effective in reducing tumor formation or metastasis.

Conventional methods, known to those of ordinary skill in the art of medicine, may be used to administer the anti-cancer therapeutic agent to the subject, depending upon the type of disease to be treated or the site of the disease. The anti-cancer therapeutic agent can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, an anti-cancer therapeutic agent may be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (e.g., glycerol, propylene glycol, liquid polyethylene glycol, and the like). For intravenous injection, water soluble anti-cancer therapeutic agents can be administered by the drip method, whereby a pharmaceutical formulation containing the antibody and a physiologically acceptable excipients is infused. Physiologically acceptable excipients may include, for example, 5% dextrose, 0.9% saline, Ringer's solution, and/or other suitable excipients. Intramuscular preparations, e.g., a sterile formulation of a suitable soluble salt form of the anti-cancer therapeutic agent, can be dissolved and administered in a pharmaceutical excipient such as Water-for-Injection, 0.9% saline, and/or 5% glucose solution.

In one embodiment, an anti-cancer therapeutic agent is administered via site-specific or targeted local delivery techniques. Examples of site-specific or targeted local delivery techniques include various implantable depot sources of the agent or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568, the contents of each of which are incorporated by reference herein for this purpose.

In some embodiments, more than one anti-cancer therapeutic agent, such as an antibody and a small molecule inhibitory compound, may be administered to a subject in need of the treatment. The agents may be of the same type or different types from each other. At least one, at least two, at least three, at least four, or at least five different agents may be co-administered. Generally anti-cancer agents for administration have complementary activities that do not adversely affect each other. Anti-cancer therapeutic agents may also be used in conjunction with other agents that serve to enhance and/or complement the effectiveness of the agents.

Treatment efficacy can be assessed by methods well-known in the art, e.g., monitoring tumor growth or formation in a patient subjected to the treatment. Alternatively, or in addition to, treatment efficacy can be assessed by monitoring tumor type over the course of treatment (e.g., before, during, and after treatment).

A subject having cancer may be treated using any combination of anti-cancer therapeutic agents or one or more anti-cancer therapeutic agents and one or more additional therapies (e.g., surgery and/or radiotherapy). The term combination therapy, as used herein, embraces administration of more than one treatment (e.g., an antibody and a small molecule or an antibody and radiotherapy) in a sequential manner, that is, wherein each therapeutic agent is administered at a different time, as well as administration of these therapeutic agents, or at least two of the agents or therapies, in a substantially simultaneous manner.

Sequential or substantially simultaneous administration of each agent or therapy can be affected by any appropriate route including, but not limited to, oral routes, intravenous routes, intramuscular, subcutaneous routes, and direct absorption through mucous membrane tissues.

The agents or therapies can be administered by the same route or by different routes. For example, a first agent (e.g., a small molecule) can be administered orally, and a second agent (e.g., an antibody) can be administered intravenously.

As used herein, the term “sequential” means, unless otherwise specified, characterized by a regular sequence or order, e.g., if a dosage regimen includes the administration of an antibody and a small molecule, a sequential dosage regimen could include administration of the antibody before, simultaneously, substantially simultaneously, or after administration of the small molecule, but both agents will be administered in a regular sequence or order. The term “separate” means, unless otherwise specified, to keep apart one from the other. The term “simultaneously” means, unless otherwise specified, happening or done at the same time, i.e., the agents are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents or therapies described herein.

Combination therapy can also embrace the administration of the anti-cancer therapeutic agent (e.g., an antibody) in further combination with other biologically active ingredients (e.g., a vitamin) and non-drug therapies (e.g., surgery or radiotherapy).

It should be appreciated that any combination of anti-cancer therapeutic agents may be used in any sequence for treating a cancer. The combinations described herein may be selected on the basis of a number of factors, which include but are not limited to reducing tumor formation or tumor growth, and/or alleviating at least one symptom associated with the cancer, or the effectiveness for mitigating the side effects of another agent of the combination. For example, a combined therapy as provided herein may reduce any of the side effects associated with each individual members of the combination, for example, a side effect associated with an administered anti-cancer agent.

In some embodiments, an anti-cancer therapeutic agent is an antibody, an immunotherapy, a radiation therapy, a surgical therapy, and/or a chemotherapy.

Examples of the antibody anti-cancer agents include, but are not limited to, alemtuzumab (Campath), trastuzumab (Herceptin), trastuzumab deruxtecan (Enhertu), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor (e.g., nivolumab (Opdivo), pembrolizumab (Keytruda), atezolizumab (Tecentriq), avelumab (Bavencio), durvalumab (Imfinzi)), a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, Carboplatin or Cisplatin, Docetaxel, Gemcitabine, Nab-Paclitaxel, Paclitaxel, Pemetrexed, and Vinorelbine.

Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

### Machine Learning

In some embodiments, at least one trained machine learning classifier is used to determine whether a tumor sample has a particular breast cancer molecular subtype. In some embodiments, a first trained machine learning classifier is used to determine whether a tumor sample has a basal molecular subtype. In some embodiments, a second trained machine learning classifier is used to determine whether a tumor sample has a HER2-high molecular subtype. In some embodiments, a third trained machine learning classifier is used to determine whether a tumor sample has a HER2-low molecular subtype. In some embodiments, a fourth trained machine learning classifier is used to determine whether a tumor sample has a Luminal A or Luminal B molecular subtype.

In some embodiments, a machine learning classifier is a generalized linear model (e.g., a logistic regression model, a probit regression model, etc.), a support vector machine, a decision tree model, a gradient-boosted decision tree model, a Gaussian mixture model, a random forest model, a neural network model, or any other suitable type of machine learning model, as aspects of the technology described herein are not limited in this respect. For example, the at least one machine learning classifier may be a gradient-boosted decision tree classifier.

In some embodiments, each of the machine learning classifiers described herein is the same type of machine learning classifier. For example, all of the machine learning classifiers may be gradient-boosted decision tree classifiers. In some embodiments, the machine learning classifiers may be different types. For example, with reference to FIG. 1C, the first, second, and third machine learning classifiers 110-1, 110-2, and 110-3 may be gradient boosted decision tree classifiers, while the fourth machine learning classifier 110-4 may be a logistic regression model.

In some embodiments, the machine learning model(s) may be trained using any suitable training technique(s), including supervised techniques, semi-supervised techniques, unsupervised techniques, or any suitable combination thereof without limitation.

In some embodiments, a decision tree classifier may be used. Any suitable type of decision tree classifier may be used and may be trained using any suitable supervised decision tree learning technique. For example, the decision tree classifier may be trained by the iterative dichotomizer technique (e.g., the ID3 algorithm as described, for example, in Quinlan, J. R. 1986. Induction of Decision Trees. Mach. Learn. 1, 1 (Mar. 1986), 81-106)), the C4.5 technique (e.g., as described, for example, in Quinlan, J. R. C4.5: Programs for Machine Learning. Morgan Kaufmann Publishers, 1993), the classification and regression tree (CART) technique (e.g., as described, for example, in Breiman, Leo; Friedman, J. H.; Olshen, R. A.; Stone, C. J. (1984). Classification and regression trees. Monterey, CA: Wadsworth & Brooks/Cole Advanced Books & Software). It should be appreciated that a decision tree classifier may be trained using any other suitable training method, without limitation.

In some embodiments, a gradient-boosted decision tree classifier may be used. The gradient-boosted decision tree classifier may be an ensemble of multiple decision tree classifiers (sometimes called “weak learners”). The prediction (e.g., classification) generated by the gradient-boosted decision tree classifier is formed based on the predictions generated by the multiple decision trees part of the ensemble. The ensemble may be trained using an iterative optimization technique involving calculation of gradients of a loss function (hence the name “gradient” boosting). Any suitable supervised training algorithm may be applied to training a gradient-boosted decision tree classifier including, for example, any of the algorithms described in Hastie, T.; Tibshirani, R.; Friedman, J. H. (2009). “10. Boosting and Additive Trees”. The Elements of Statistical Learning (2nd ed.). New York: Springer. pp. 337-384. In some embodiments, the gradient-boosted decision tree classifier may be implemented using any suitable publicly available gradient boosting framework such as XGBoost (e.g., as described, for example, in Chen, T., & Guestrin, C. (2016). XGBoost: A Scalable Tree Boosting System. In Proceedings of the 22nd ACM SIGKDD International Conference on Knowledge Discovery and Data Mining (pp. 785-794). New York, NY, USA: ACM.). The XGBoost software may be obtained from http://xgboost.ai, for example). Another example framework that may be employed is LightGBM (e.g., as described, for example, in Ke, G., Meng, Q., Finley, T., Wang, T., Chen, W., Ma, W., . . . Liu, T.-Y. (2017). Lightgbm: A highly efficient gradient boosting decision tree. Advances in Neural Information Processing Systems, 30, 3146-3154). The LightGBM software may be obtained from https://lightgbm.readthedocs.io/, for example).

In some embodiments, a neural network classifier may be used. The neural network classifier may be trained using any suitable neural network optimization software. The optimization software may be configured to perform neural network training by gradient descent, stochastic gradient descent, or in any other suitable way. In some embodiments, the Adam optimizer (Kingma, D. and Ba, J. (2015) Adam: A Method for Stochastic Optimization. Proceedings of the 3rd International Conference on Learning Representations (ICLR 2015)) may be used.

In some embodiments, a support vector machine (SVM) may be used. The SVM may be implemented using any suitable techniques such as, for example, any of the techniques described by Cristianini, N., and Shawe-Taylor, J. (“An introduction to support vector machines and other kernel-based learning methods.” Cambridge university press, 2000), which is incorporated by reference herein in its entirety.

In some embodiments, a Gaussian mixture model may be used. The Gaussian mixture model may be implemented using any suitable techniques such as, for example, any of the techniques described by Reynolds, D. (“Gaussian mixture models.” Encyclopedia of biometrics 741.659-663 (2009)), which is incorporated by reference herein in its entirety.

In some embodiments, a random forest model may be used. The random forest model may be implemented using any suitable techniques such as, for example, any of the techniques described by Biau, G. (“Analysis of a random forests model.” The Journal of Machine Learning Research 13.1 (2012): 1063-1095), which is incorporated by reference herein in its entirety.

In some embodiments, a generalized linear model may be used. The generalized linear model may be trained by fitting the model to training data to estimate parameters for the model. In some embodiments, the parameters are estimated using maximum likelihood estimation (MLE). In some embodiments, the parameters are estimated using Bayesian estimation. However, it should be appreciated that any suitable parameter estimation techniques may be used, as aspects of the technology described herein are not limited in this respect.

### Computer Implementation

An illustrative implementation of a computer system 1400 that may be used in connection with any of the embodiments of the technology described herein (e.g., such as the process 200 shown in FIG. 2) is shown in FIG. 14. The computer system 1400 includes one or more processors 1410 and one or more articles of manufacture that comprise non-transitory computer-readable storage media (e.g., memory 1420 and one or more non-volatile storage media 1430). The processor 1410 may control writing data to and reading data from the memory 1420 and the non-volatile storage media 1430 in any suitable manner, as the aspects of the technology described herein are not limited to any particular techniques for writing or reading data. To perform any of the functionality described herein, the processor 1410 may execute one or more processor-executable instructions stored in one or more non-transitory computer-readable storage media (e.g., the memory 1420), which may serve as non-transitory computer-readable storage media storing processor-executable instructions for execution by the processor 1410.

Computing system 1400 may include a network input/output (I/O) interface 1440 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing system 1400 may also include one or more user I/O interfaces 1450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

### EXAMPLES

### Example 1

This disclosure describes a transcriptomic classification system that can identify subjects having HER2-low breast cancers (BCs). Accurate identification of HER2-low BCs may help physicians navigate treatment selection and, in turn, allow patients to benefit from targeted therapies for HER2-low breast cancers (e.g., trastuzumab deruxtecan).

Here, 6,361 BC samples with clinical and pathological annotation, including FISH and IHC data, from three public datasets (TCGA BRCA and SCAN-B, [total n=4,457]; and METABRIC, [n=1904]) were analyzed for gene expression, differential expression, gene enrichment, copy number alterations, and mutations.

An algorithm was first used to categorize samples from TCGA and SCAN-B into molecular types by customizing a gene set, employing UMAP, and utilizing clustering with HDBSCAN. Each resulting cluster associated with a specific gene set was named and excluded from the next step of analysis. Examples of gene sets used to categorize samples are shown in Tables 3 and 4. After four iterations, five molecular BC types were sequentially identified: Basal, HER2-high, HER2-low, Luminal B (LumB), and Luminal A (LumA), as shown in FIG. 3-1 and FIG. 3-2.

FIG. 3-1 and FIG. 3-2 show a representative heatmap of 4,457 TCGA and SCAN-B carcinomas segregated into the five breast cancer molecular types (Basal, HER2-high, HER2-low, Luminal A (Lum A), and Luminal B (Lum B) types) by a hierarchical classifier model (e.g., a Light GBM-based classifier).

TABLE 3

| | | | | | | |
|---|---|---|---|---|---|---|
| CCNE1 | GPR160 | ACTR3B | VIM | FGFR4 | MCM2 | ZNF608 |
| ANLN | ESR1 | LBP | UBE2T | BLVRA | BUB1 | ZCCHC24 |
| TWIST1 | TFAP2B | BAG1 | MMP11 | CLDN8 | E2F1 | AASS |
| NUF2 | CDC6 | BCL2 | KRT5 | EGFR | MYBL2 | |
| ERBB2 | GRB7 | CDH3 | CTNNA1 | MYC | GNG12 | |
| MAPT | ELF5 | PPARG | KRT17 | CCNB1 | SOCS5 | |
| MLPH | CDC20 | MDM2 | KIT | UBE2C | CRY2 | |
| SOX10 | FOXA1 | MKI67 | CAV1 | AURKB | ELN | |
| PHGDH | FOXC1 | FABP7 | KRT14 | AURKA | PTPN21 | |
| AR | PGR | WNK4 | FABP4 | PLK1 | COL14A1 | |

TABLE 4

| | | | | |
|---|---|---|---|---|
| UBE2T | RRM2 | ACTR3B | ESR1 | BLVRA |
| BIRC5 | UBE2C | MYC | SLC39A6 | MMP11 |
| NUF2 | CENPF | EGFR | MAPT | FGFR4 |
| CDC6 | PTTG1 | KRT5 | PGR | GRB7 |
| CCNB1 | EXO1 | PHGDH | CXXC5 | ERBB2 |
| TYM5 | ANLN | CDH3 | MLPH | |
| MYBL2 | CCNE1 | KRT17 | BCL2 | |
| CEP55 | CDC20 | FOXC1 | MDM2 | |
| MELK | MKI67 | SFRP1 | NAT1 | |

| | | | |
|---|---|---|---|
| NDC80 | KIF2C | KRT14 | FOXA1 |

A hierarchical classifier model (e.g., a Light GBM-based model) was then trained and validated on the TCGA and SCAN-B cohorts, which were mixed and split into a training set and a validation set. Samples with high ERBB2 expression and copy number amplification were defined as HER2-high, and samples with similar PAM50 expression profiles but lacking ERBB2 expression were defined as HER2-low. Luminal samples were grouped into LumA and LumB types based on their proliferation rates: low for LumA and high for LumB.

Mutational and gene expression profiles of HER2-high, Basal, LumA, and LumB molecular BC types were investigated. ERBB2 copy number amplification and high gene expression was observed in HER2-high molecular BC type samples from the TCGA and METABRIC cohorts, as confirmed by FISH and IHC in 97% and 93% of samples, respectively. Conversely, high ERBB2 expression or copy number amplification was not observed in the HER2-low molecular BC type samples, which was concordant with an absence of HER2 3+ expression by IHC. HER2-low molecular BC type samples had high mutational frequencies in PTEN, KMT2C, and ERBB2 (12%, 14%, and 8% respectively, adj. p<0.005, chi-square test). HER2-low molecular BC type samples also showed increased EGFR (log FC=2.8, adj. p<0.001) and CLDN8 (log FC=3.4, adj. p<0.001) expression levels. The classifier identified 55% of the HER2-low molecular BC type samples as the luminal androgen receptor (LAR) Burstein subtype of BC, as a result of higher AR expression than in HER2-high molecular BC type samples (log FC=2.9, p=0.04). The classification of SCAN-B luminal samples into the LumA and LumB molecular BC types also better predicted Ki-67 positivity (≥20% of Ki-67+ malignant cells by IHC, F1-score=0.77) than the previously reported PAM50 classification (F1-score=0.62).

The classifier described in this disclosure effectively defined five molecular breast cancer types, including the HER2-low molecular BC type, based on their molecular profiles. By determining HER2 status with transcriptomic data, the classifier can help oncologists make more precise treatment decisions for targeted therapy by minimizing IHC-related reproducibility issues. Given its concordance with copy number amplification, IHC, and FISH data, the classifier can precisely identify the HER2 status of BCs. Since HER2-low molecular type BCs may represent a unique group that requires different treatment approaches, the classifier may assist in therapeutic decision-making because HER2-low is an essential biomarker to guide treatment selection. For example, the DESTINY-Breast04 trial recently demonstrated the benefit of treating patients with HER2-low metastatic BC with the anti-HER2 antibody-drug conjugate (ADC) trastuzumab deruxtecan (T-DXd). To date, three anti-HER2 ADCs have proven activity in HER2-low BC: trastuzumab deruxtecan (T-DXd), trastuzumab duocarmazine (SYD985), and Disitamab vedotin (RC48). In some embodiments, a subject is administered an anti-HER2 ADC (e.g., T-DXd, SYD985, RC48) based upon a determination of the subject's molecular BC type using a classifier as described by this disclosure.

### Example 2

This example describes a transcriptomic analysis technique for identifying biological subtypes of breast cancer (BC). Analysis of 6,223 clinically and pathologically annotated breast cancer samples was conducted from three publicly-available datasets (TCGA BRCA and SCAN-B [n=4,319], and METABRIC [n=1,904)). Datasets included FISH and IHC data. Gene expression, differential expression, gene enrichment, copy number alterations, and mutations were analyzed for the datasets. A Light GBM-based hierarchical classifier model was applied to categorize samples from TCGA and SCAN-B into subtypes by customizing a gene set, employing UMAP dimensionality reduction, and clustering with a HDBSCAN technique. Examples of gene used to categorize samples are shown in Table 5. Each resulting cluster that was associated with a specific gene set was named and excluded from subsequent analysis. After four iterations, five molecular BC types were sequentially identified: Basal, HER2-high, HER2-low, Luminal B (LumB), and Luminal A (LumA). The five identified subtypes were observed to overlap with the PAM50 system (and may be referred to herein as “PAM50(BG)” classification). The METABRIC dataset was used to validate the Light GBM-based hierarchical classifier model.

TABLE 5

| | | | | |
|---|---|---|---|---|
| FOXA1 | MLPH | ESR1 | FOXC1 | MYC |
| PHGDH | ACTR3B | CDH3 | SFRP1 | KRT17 |
| KRT14 | KRT5 | EGFR | CDC6 | FGFR4 |
| MAPT | CXXC5 | BCL2 | PGR | NAT1 |
| SLC39A6 | MDM2 | MMP11 | BLVRA | CCNE1 |
| MYBL2 | RRM2 | ORC6 | NUF2 | NDC80 |
| TVMS | CCNB1 | ANLN | UBE2T | CENPF |
| EXO1 | MKI67 | BIRC5 | PTTG1 | UBE2C |
| CDC20 | KIF2C | CEP55 | MELK | GRB7 |
| ERBB2 | ACE2 | FABP7 | AKR1B15 | GATA3 |

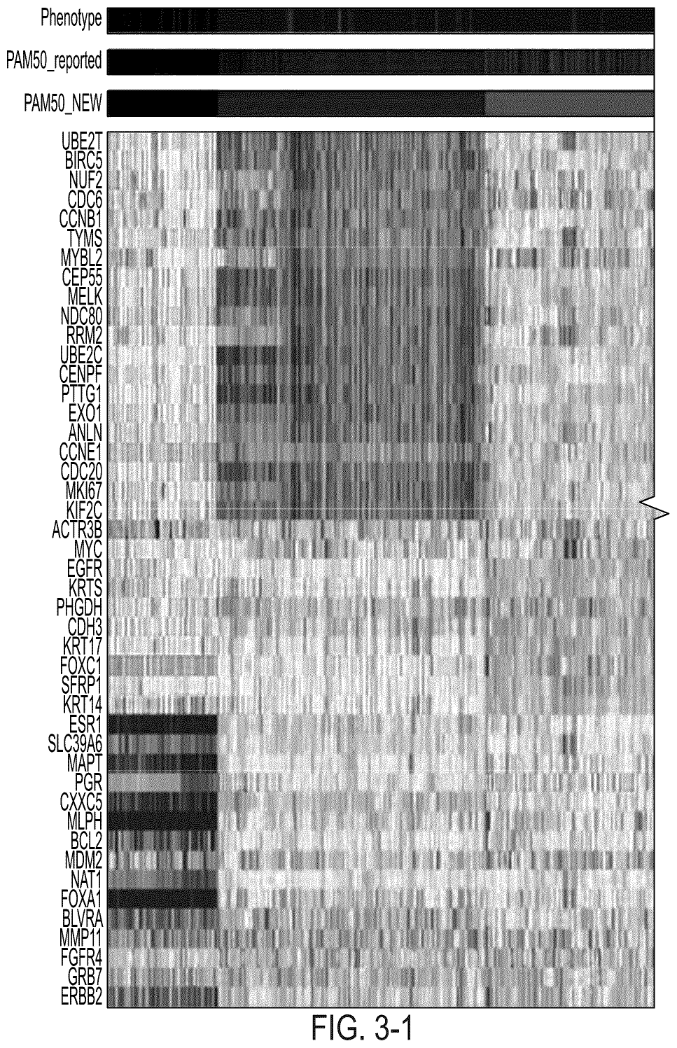

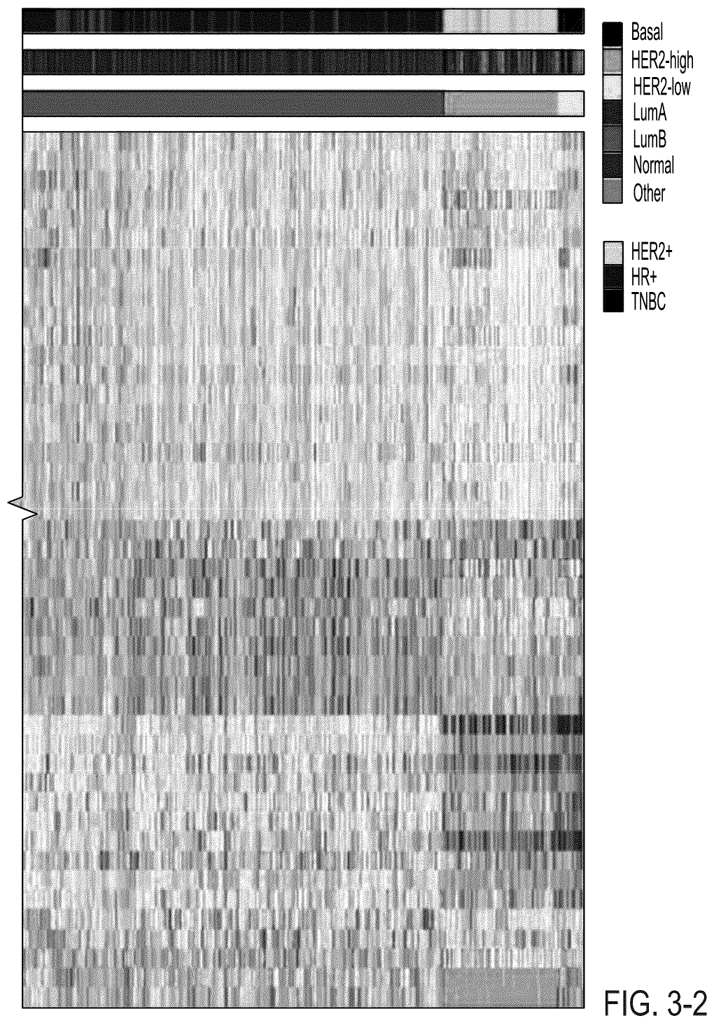

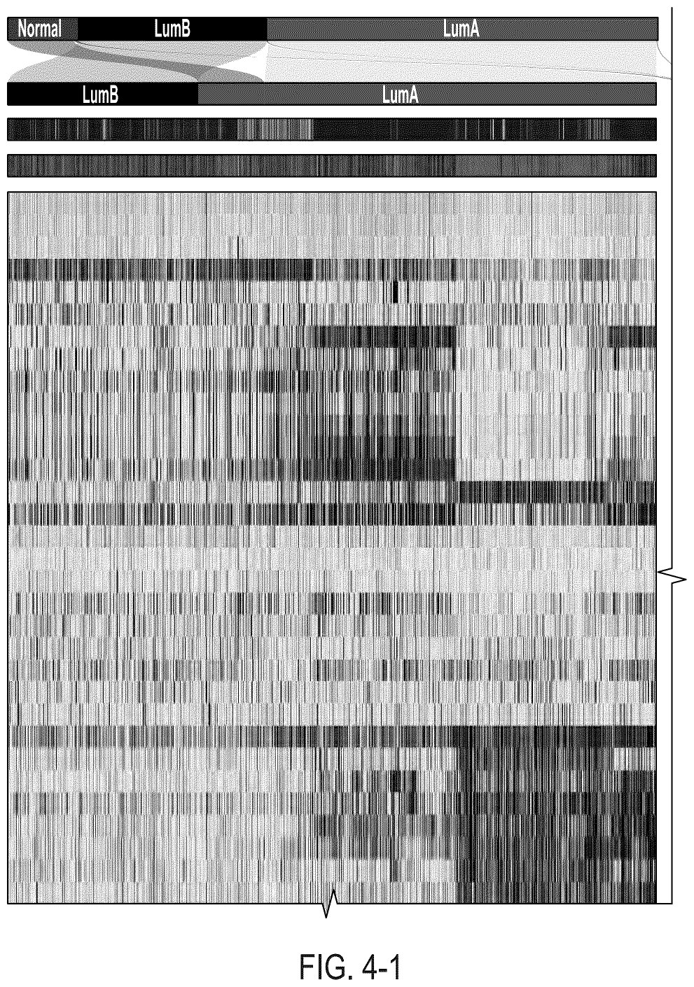

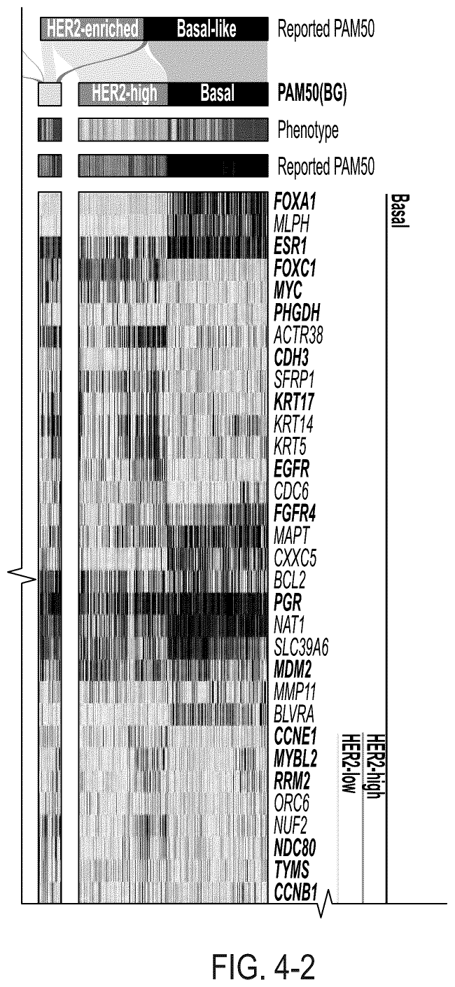

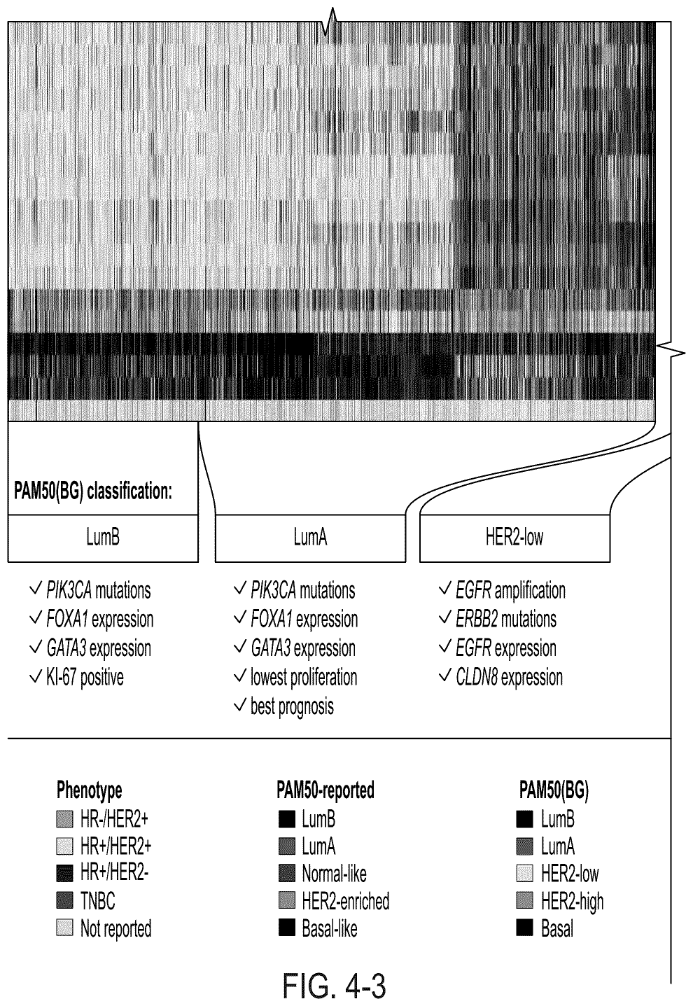

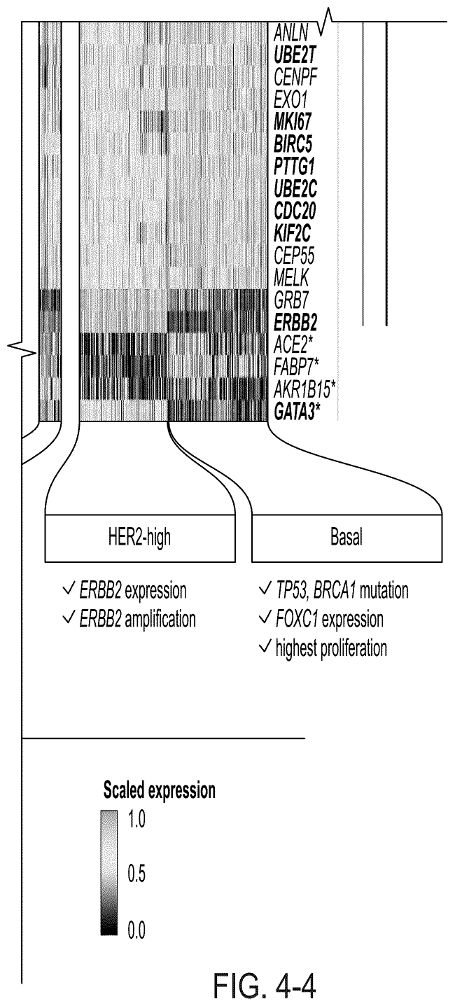

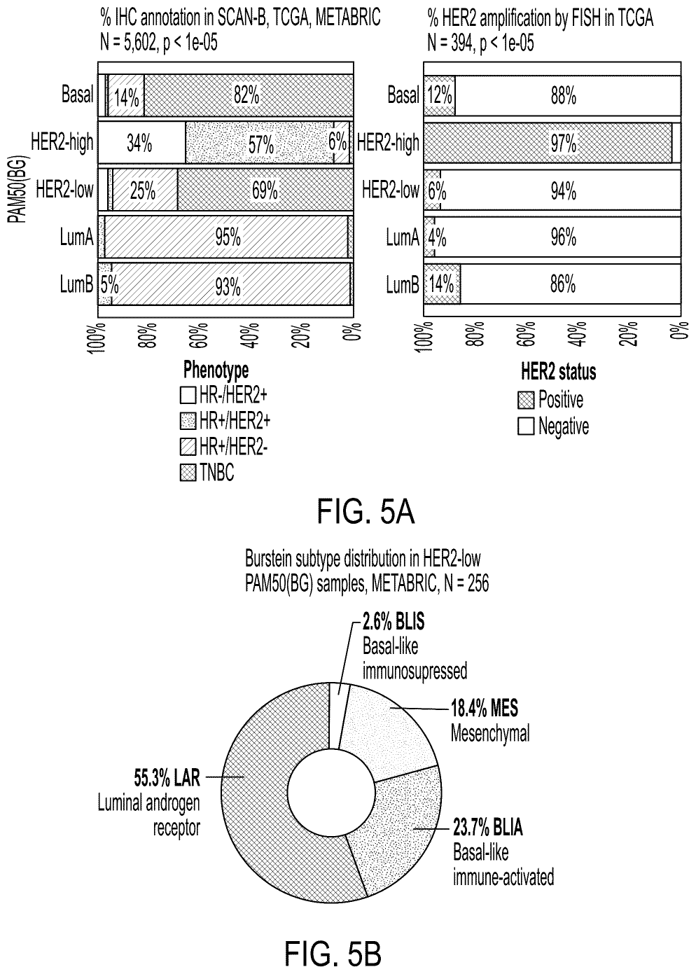

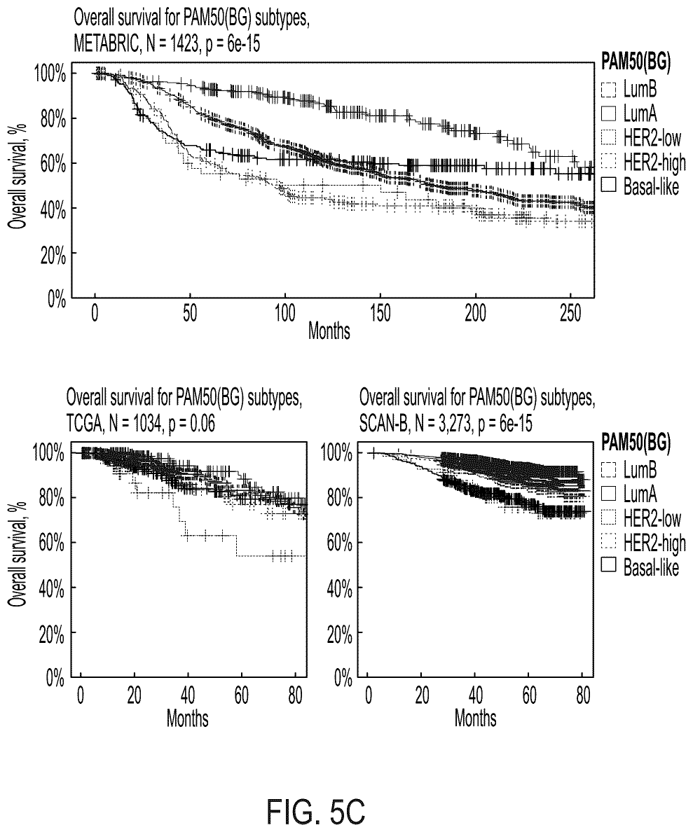

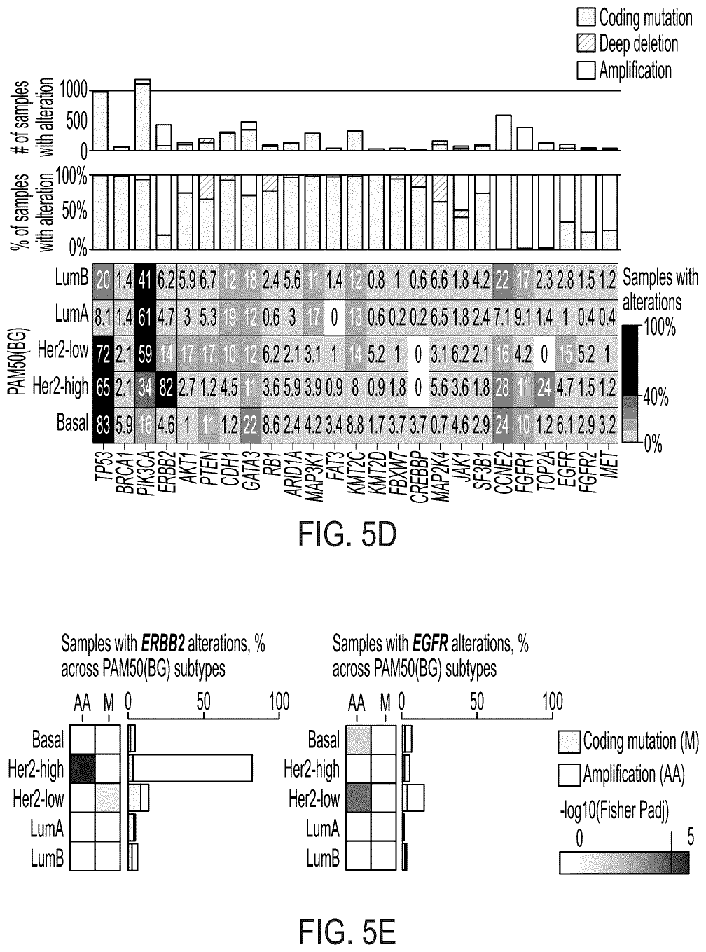

A representative heatmap describing the five molecular BC type clusters is shown in FIG. 4-1, FIG. 4-2, FIG. 4-3, and FIG. 4-4. The Basal molecular BC type is characterized by TP53 and BRCA mutations, high FOXC1 expression (relative to other molecular BC types), and the highest proliferation rates of all the molecular BC types. The HER2-high molecular BC type is characterized by ERBB2 amplification, and high ERBB2 expression (relative to other molecular BC types). HER2-high samples were confirmed by IHC and FISH in 91% and 97% of samples, respectively (FIG. 5A). The HER2-low molecular BC type is characterized by EGFR amplification, ERBB2 mutation, and high EGFR and CLDN8 expression (relative to other molecular BC types). The Luminal A (LumA) molecular BC type is characterized by a low proliferation rate relative to other molecular BC types. The Luminal B (LumB) molecular BC type is characterized by a high proliferation rate relative to other molecular BC types.

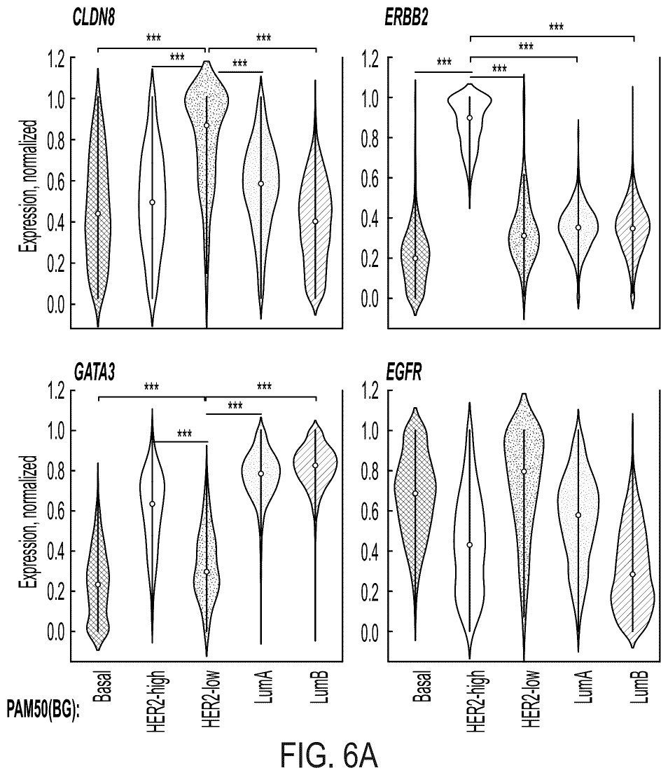

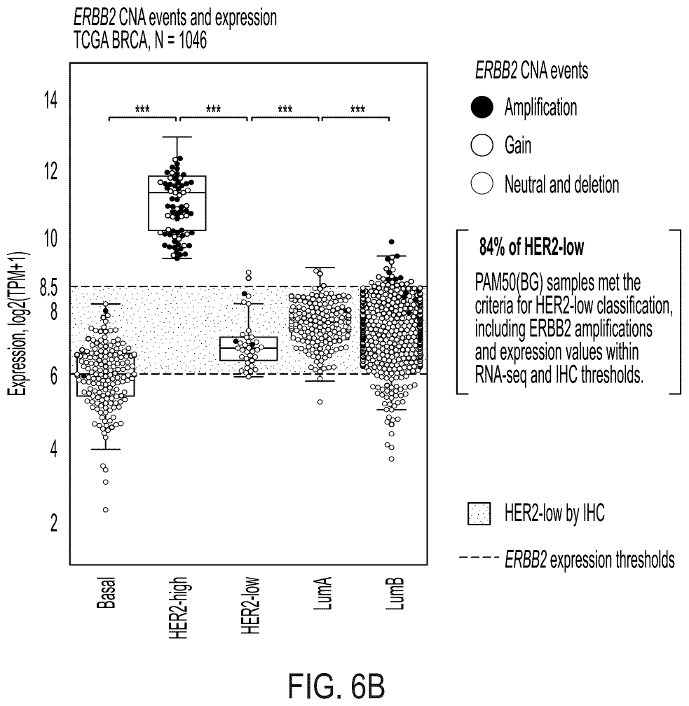

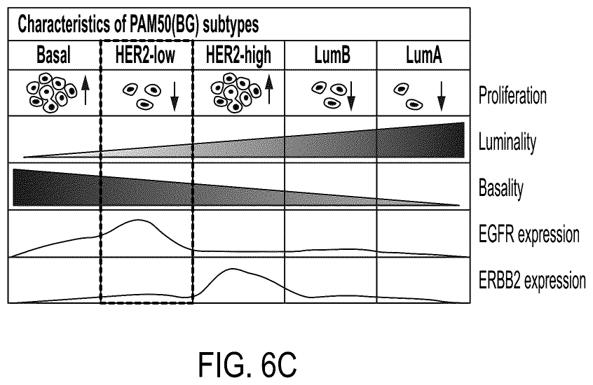

The classifier identified 66% of the samples with a luminal androgen receptor (LAR) Burstein type as HER2-low molecular BC type (FIG. 5B). Survival analysis indicates that the HER2-low molecular BC type consistently exhibits the poorest treatment outcomes across METABRIC, TCGA, and SCAN-B datasets (FIG. 5C). HER2-low molecular BC type samples also had frequent coding mutations in TP53 (72%, p=1e-13), PI3CA (53%, p=0.02), ERBB2 (8%, p=0.05), KMT2D (5%, p=0.01), and PTEN (11%, p=6e-5), as well as frequent copy number amplifications in EGFR (11%, p=6e-5) and AKT1 (9%, p=1e-5), as shown in FIGS. 5D and 5E). Additionally, the HER2-low molecular BC type was associated with high EGFR and CLDN8 (padj=<0.001) and low ERBB2 and GATA3 expression (padj=<0.001), as shown in FIG. 6A. The HER2-high, Basal, LumA, and LumB molecular BC types exhibited mutational and gene expression profiles consistent with PAM50 analysis. ERBB2 expression fell within RNA-seq thresholds previously observed for 92% of HER2-low patients, and only 6% were observed to have ERBB2 amplifications (FIG. 6B). The HER2-low molecular BC type also exhibited low proliferation, moderately high basality, high EGFR expression and low ERBB2 expression (FIG. 6C).

In summary, the molecular BC type classifier described in this example offers a more refined categorization of BCs than previously available techniques, for example PAM50.

Subjects identified as having HER2-low molecular BC type may benefit from administration of HER2-targeted therapies, for example T-DXd.

### Example 3

This example describes characterization of breast cancers into intrinsic subtypes through hierarchical clustering analyses of gene expression. The classification, based on 50 gene expression profiles (e.g., PAM50), led to the identification of five subtypes: Luminal A, Luminal B, HER2-enriched, Basal, and Normal-like. These subtypes have demonstrated prognostic significance across varying patient cohorts, both untreated and those treated with tamoxifen, thus showcasing their potential in risk stratification.

The Prosigna test emerged as a consequential extension of this classification, aiding in the clinical management of ER+/HER2− early-stage breast cancer by categorizing patients into low, intermediate, and high-risk groups. Moreover, it identified intrinsic subtypes indicative of late recurrence tendencies. However, despite its clinical assimilation and high reproducibility, the 50-gene expression profile classification harbors certain gray zones, an aspect that recent publications have delineated, emphasizing the need for more nuanced classifications.

Recent studies have shed light on a distinct subtype known as the HER2-low subtype, reflecting a unique molecular and clinical entity. Investigations have reported the prevalence of HER2-low expression, particularly in estrogen-receptor and progesterone receptor positive subtypes of breast cancer and compared different assays' effectiveness in determining HER2 status. Molecular characteristics and prognostic analyses associated with HER2-low breast cancer have indicated that understanding PAM50 intrinsic subtypes within HER2-low may explain different clinical behaviors and responses to treatment. Moreover, Immunohistochemistry (IHC) has revealed HER2-low-positive tumors as a new subgroup of breast cancer, distinct from HER2-zero tumors, with unique biology and varying responses to therapy, particularly in therapy-resistant, hormone receptor-negative tumors. The evolution of the HER2-positive subtype, following the introduction of HER-targeted therapies, has further highlighted the benefit of these therapies, especially in cases of HER2 overexpression or gene amplification. The recent FDA approval of the first treatment targeting low levels of HER2 in HER2-low breast cancer indicates the clinical recognition and potential treatment advancements for this subtype. Emerging evidence from clinical trials challenges the binary categorization of HER2 status in breast cancers, showcasing significant clinical benefits in advanced breast cancers with “HER2-low” expression associated with the use of HER2-directed Antibody-Drug Conjugates (ADCs).

This example describes a rank-based classifier to conduct intrinsic subtyping in either single samples or unbalanced cohorts. Utilizing advanced clustering techniques and machine learning algorithms, a comparative assessment was performed of the prognostic aptitudes between the traditional PAM50 intrinsic subtypes and the newly defined five subtypes. The analysis ventured beyond the conventional IHC methodology to unravel the HER2-low subtype as a distinct biological entity using RNA-sequencing and Whole exome sequencing. This endeavor revealed that the conventional HER2-enriched classification does not exclusively reflect HER2 amplification, and that IHC 1+ and 2+ with FISH amplification fell short in encapsulating the biological complexity of the HER2-low subtype. Unlike its depiction in IHC terms, the data indicates the HER2-low subtype is a unique biological classification, thus enriching the understanding of HER2 status in breast cancer molecular taxonomy.

### Material and Methods

### Datasets

Publicly available gene expression datasets with clinical annotation: TCGA, SCAN-B, Molecular Taxonomy of Breast Cancer International Consortium (METABRIC), were studied for subtype identification, 6223 patients in total. Datasets were collected during different years, and patients were treated according to guidelines approved for the corresponding period. All three datasets are enriched by all IHC statuses. Also TCGA and METABRIC datasets had information about mutations and amplifications. For classifier validation expressions from 15 microarray cohorts and 1 RNA-seq cohort were used (Table 6). Cohorts were selected by following criteria: all IHC phenotypes should be presented, except cohort for survival data validation (this one is TNBC dataset). Also quality of cohorts' sequencing was evaluated using PCA analysis (all outliers were disregarded for future analysis).

TABLE 6
Cohorts, used in discovery and validation of PAM50

| | | | Number of |
|---|---|---|---|
| Cohort | Status | Reference (doi) | samples |
| SCAN-B 1st cohort | Discovery cohort | 10.1186/s13073-015-0131-9 | 3273 |
| (GSE96058) | | | |
| TCGA | | 10.5114/wo.2014.47136 | 1046 |
| METABRIC | | 10.1038/nature10983 | 1904 |
| GSE102484 | Validation cohort | 10.1371/journal.pone.0184372 | 683 |
| E-MTAB-365 | Validation cohort | 10.1038/onc.2011.301 | 537 |
| SCAN-B 2nd cohort | Validation cohort | 10.1200/PO.17.00135 | 405 |
| (GSE81538) | | | |
| GSE20685 | Validation cohort | 10.1186/1471-2407-11-143 | 327 |
| GSE21653 | Validation cohort | 10.1007/s10549-010-0897-9 | 263 |
| GSE12276 | Validation cohort | 10.1038/nature08021 | 204 |
| GSE32646 | Validation cohort | 10.1111/j.1349- | 115 |
| | | 7006.2012.02231.x | |
| GSE19615 | Validation cohort | 10.1038/nm.2090 | 115 |
| CPTAC | Validation cohort | dbGaP Study Accession: | 109 |
| | | phs000892.v6.p1 | |
| GSE76360 | Validation cohort | 10.1158/1078-0432.CCR- | 100 |
| | | 15-2021 | |
| GSE66305 | Validation cohort | 10.1634/theoncologist.2015- | 86 |
| | | 0138 | |
| GSE66999 | Validation cohort | 10.1186/s40064-015-1392-x | 76 |
| GSE21974 | Validation cohort | 10.3892/or.2011.1392 | 57 |
| GSE98528 | Validation cohort | 10.1038/s41523-019-0113-y | 48 |
| GSE45581 | Validation cohort | 10.1007/s10549-013-2501-6 | 45 |
| GSE58812 | Survival | 10.1186/s13058-015-0550-y | 107 |
| | validation | | |

Datasets with reported PAM50 annotation were used for the validation of BG50 annotation. For annotation consistency Claudin-low subtype was merged with Basal, Normal-like subtype with Luminal, EGFR and HER2 subtypes were renamed as HER2-high.
WES and CNA processing

TCGA data was downloaded from GDC TCGA data portal (gdc.cancer.gov/about-data/publications/[pancanatlas or PanCanAtlas-Germline-AWG]) (10.1016/j.cels.2018.03.002). TCGA copy number alterations were evaluated with ABSOLUTE tool (10.1038/nbt.2203). Metabric fully processed was downloaded from ega-archive.org/studies/EGAS00000000083.

### Statistical Analysis and Visualization of WES Data

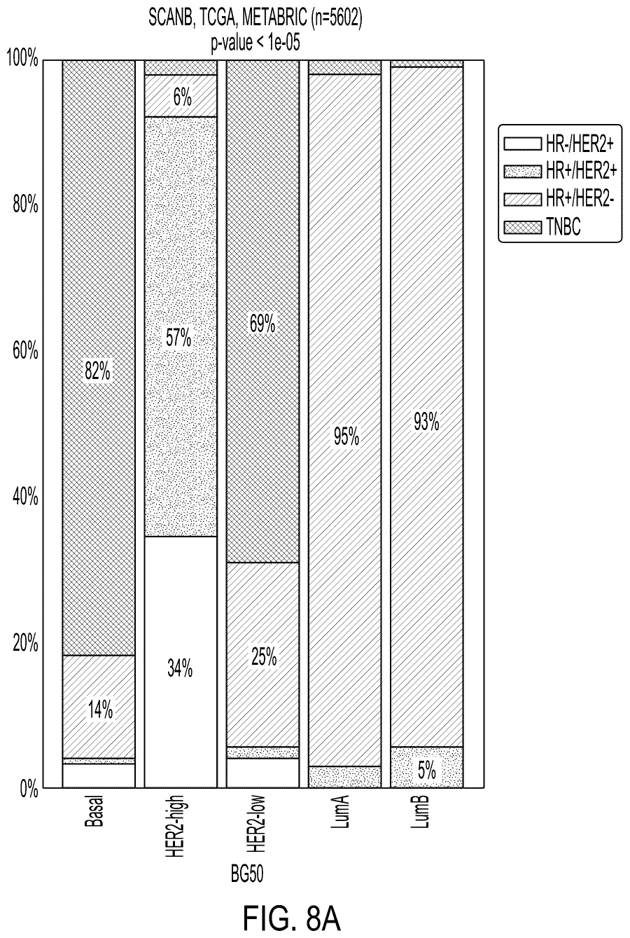

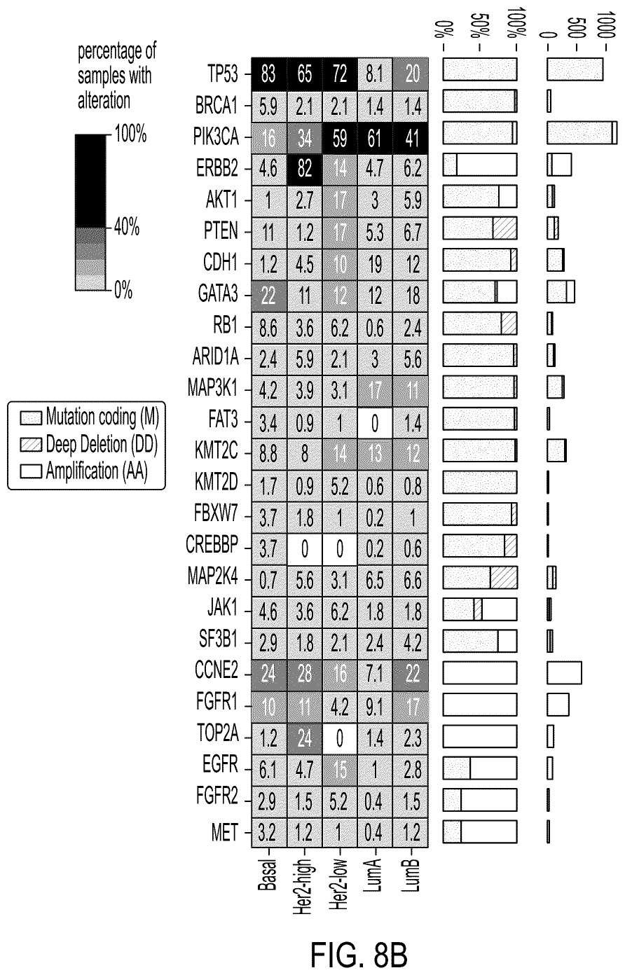

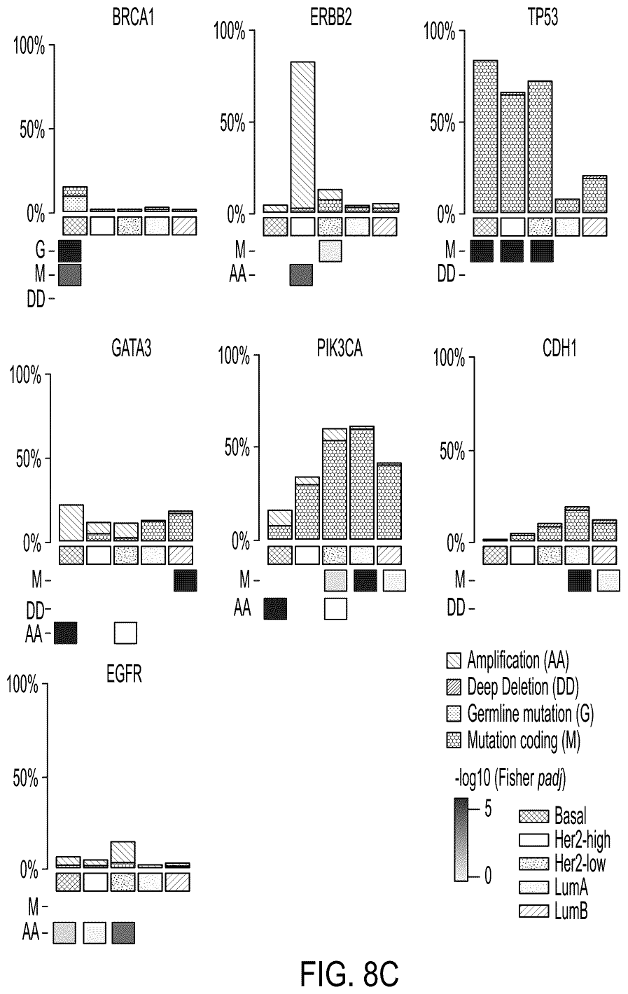

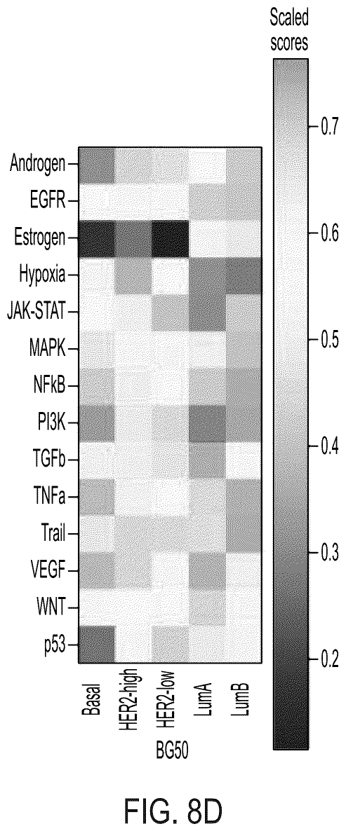

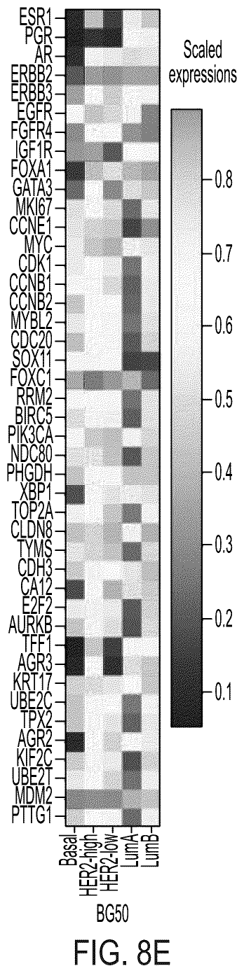

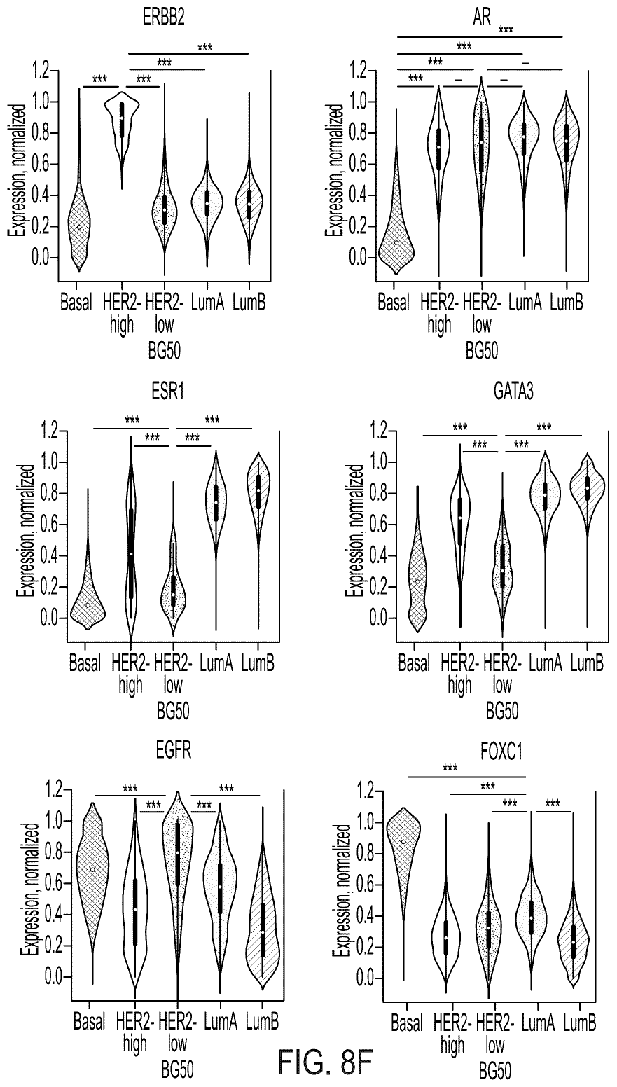

Metabric and TCGA cohorts were studied to estimate the incidence of various genetic events in different BG50 subtypes (FIG. 8B). The events of interest were: somatic mutations in coding regions, deep deletions (complete loss of a gene) and amplifications (total number of gene copies should be >=2*ploidy). Samples for which a detailed annotation was available, as well as data on any alterations of interest, were taken for analysis. To view a panorama of driver events across BG50 subtypes, all types of alterations were examined: coding mutations, amplifications, deletions (FIG. 8B). The right-tailed Fisher's exact test was used to statistically test whether a particular BG50 subtype could be characterized by the presence of a particular genetic event (somatic mutation or amplification of oncogene or deletion of oncosuppressor) (FIG. 8C). Each subtype was compared against the sum of the others. Statistics on germline mutations in BRCA1 were also included in analysis. All comparisons between different annotations (ERBB2 FISH annotation, IHC annotation) here and further were done by Pearson's chi-squared test and p-value is presented on each barplot.

### RNA-Seq Processing

TCGA transcriptomic data were downloaded from the USCS XENA portal as TPM units. SCAN-B RNA-seq data were downloaded from GEO: GSE81538 [symbol] GSE96058. Raw and processed microarray data were also downloaded from GEO.

### Statistical Analysis and Visualization of Gene Expressions Data

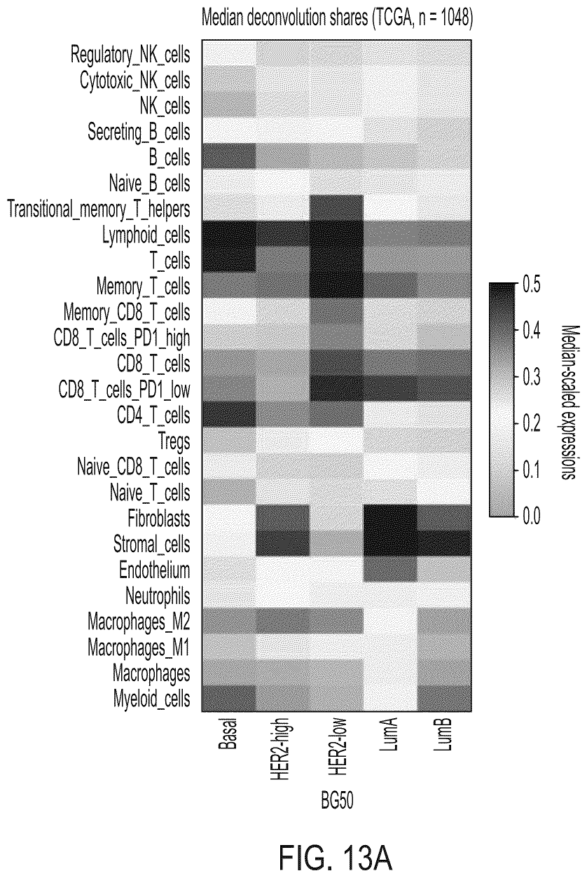

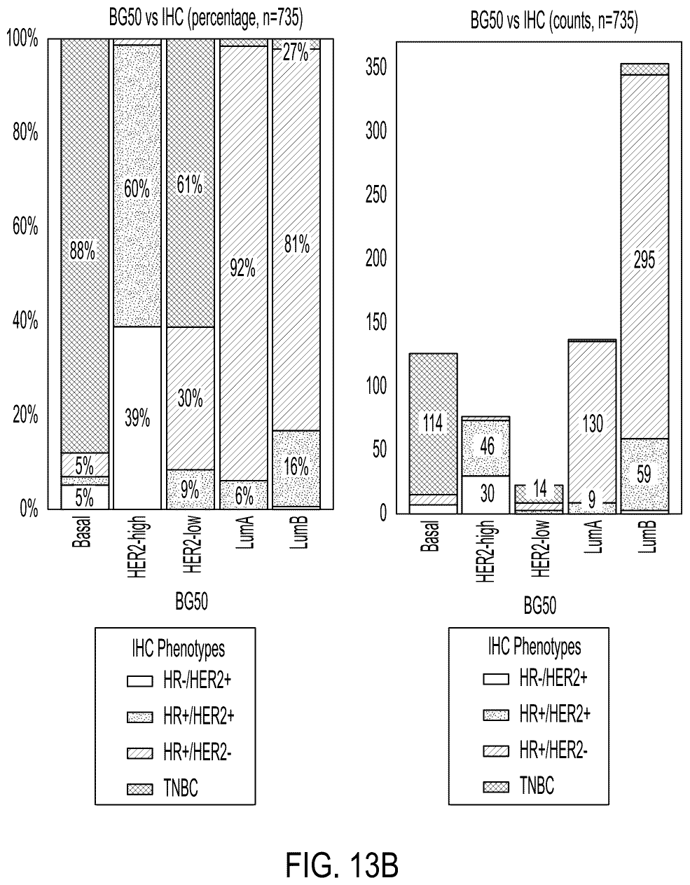

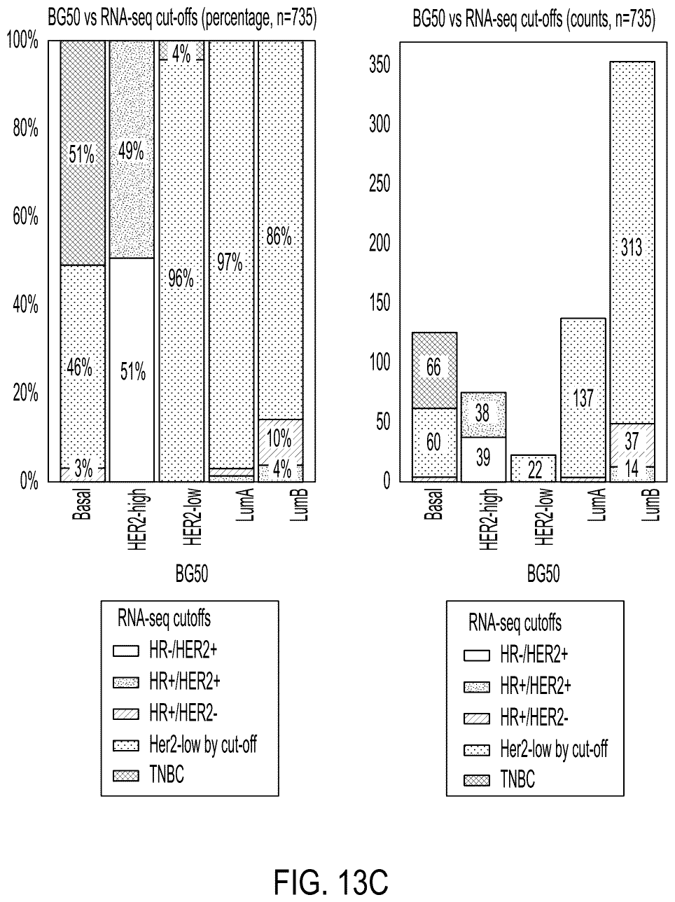

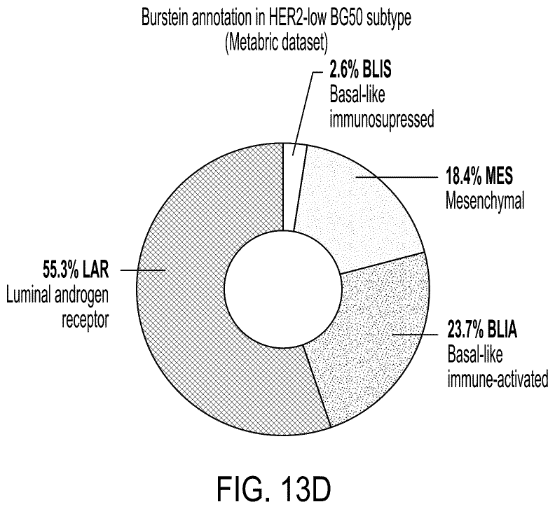

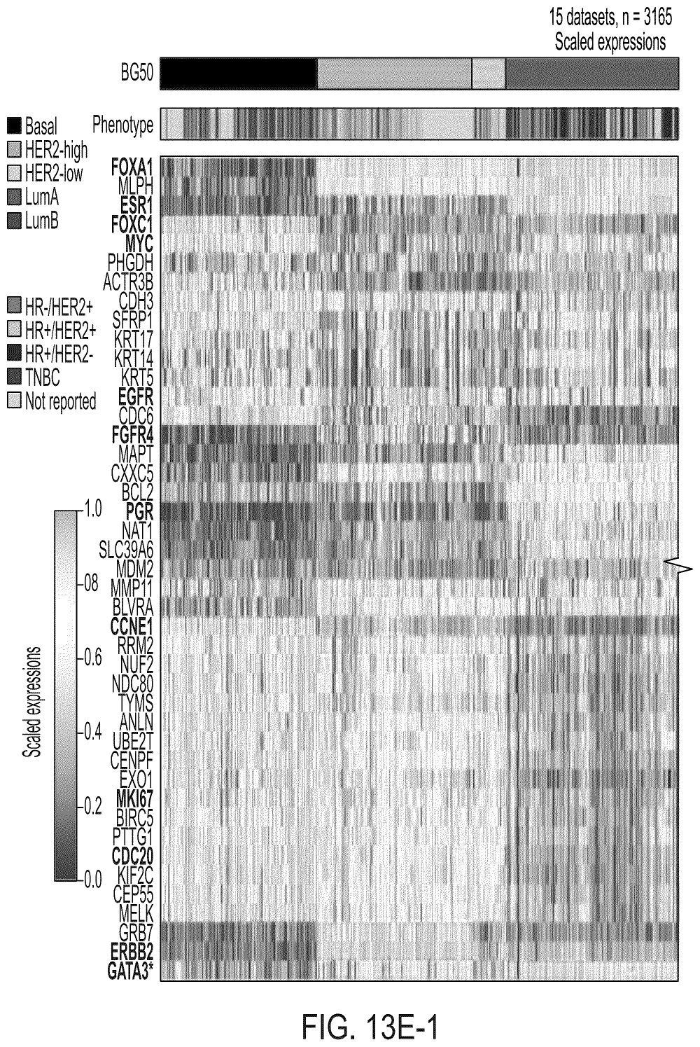

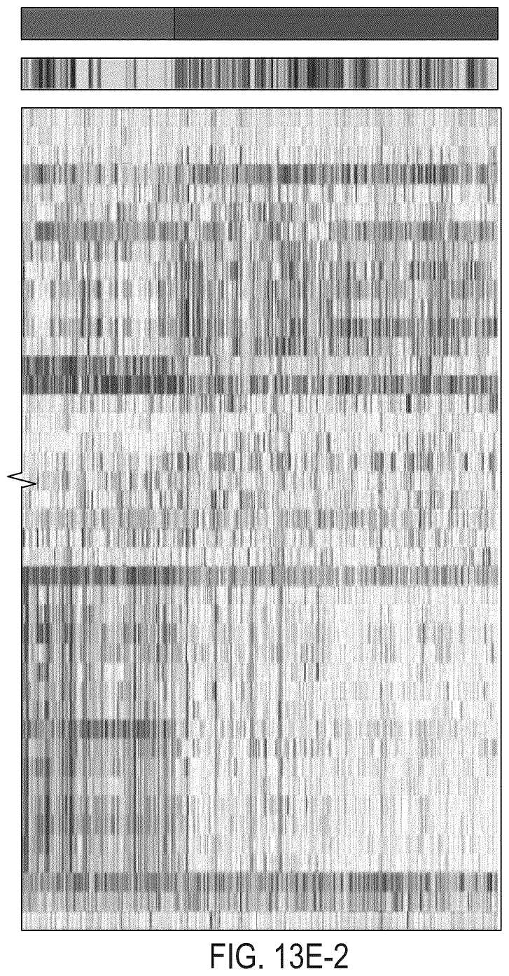

Along with expressions of single genes (FIGS. 8E, 11B, 13E-1 and 13E-2) several algorithms were applied on expression datasets. To evaluate the presence of different cell populations among BG50 subtypes, deconvolution percentages were obtained by Kassandra algorithm (10.1016/j.ccell.2022.07.006) for TCGA samples (FIG. 13A). Populations with predominantly non-null percentages in most samples were chosen for further analysis. In order to assess signaling pathways activity in different BG50 subtypes, PROGENy score (10.1038/s41467-017-02391-6) was calculated based on gene expressions of samples from Metabric, TCGA and SCANB datasets (FIG. 8D). In order to describe the identified BG50 subtypes from the perspective of selected gene expressions, cell percentages or progeny pathways, a unified algorithm was suggested. To process outliers for each feature, values which are outside 2 and 98 percentiles as lower and upper borders were trimmed subsequently in each dataset. Further features were linearly scaled from 0 to 1 independently for each dataset. Then these processed values from the datasets were merged. Values received after preprocessing on previous steps were used to compare BG50 subtypes with each other. For each feature Ad-hoc Kruskal-Wallis H-test (“stats.kruskal” function from scipy v1.9.3 Python3 library; doi: 10.1038/s41592-020-0772-5) was used to confirm that all groups were not taken from the same distribution and p-values from Dwass-Steel-Critchlow-Fligner all-pairs comparison test (“posthoc_dscf” function from scikit_posthocs v0.7.0 Python3 library; doi: 10.21105/joss.01169) were received for each scaled feature. For heatmaps in FIGS. 8D, 11B, 13A the median values of each feature for each BG50 subtype were calculated for more convenient visualization. All differentiation expression analysis were done using the limma R package (Ritchie, Matthew E., et al. “limma powers differential expression analyses for RNA-sequencing and mircroarray studies,” Nucleic acids research 43.7 (2015): e47-e47). Using the decoupler tool v. 1.3.1 (Badia-i-Mompel, Pau, et al. “decoupleR: ensemble of computational methods to infer biological activities from omics data.” Bioinformatics Advances 2.1 (2022): vbac016) transcriptional factors with high expression for each subtype were identified during subtypes description.

### Signature Construction for Normal-Like Investigation

For comparison of Normal-likes with Normal tissue and Luminal subtype adipose, proliferation and keratin signatures were used. Genes for adipose signature were taken from literature (CAV1, FABP4, PPARG, ADIPOQ, LEP, PPARG, PLIN1) (Zhu, Qingzhang, et al. “Adipocyte mesenchymal transition contributes to mammary tumor progression.” Cell reports 40.11 (2022), Kang, Taekyu, et al. “A risk-associated Active transcriptome phenotype expressed by histologically normal human breast tissue and linked to a pro-tumorigenic adipocyte population.” Breast Cancer Research 22.1 (2020): 1-15). Already published BostonGene proliferation rate signature was taken as the proliferation signature (Bagaev, Alexander, et al. “Conserved pan-cancer microenvironment subtypes predict response to immunotherapy.” Cancer cell 39.6 (2021): 845-865). Keratin signature (KRT5, KRT14, KRT15, KRT16) was constructed from literature. Scores for signatures were calculated using ES (Subramanian t al, (2005)).

### Subtypes Reannotation

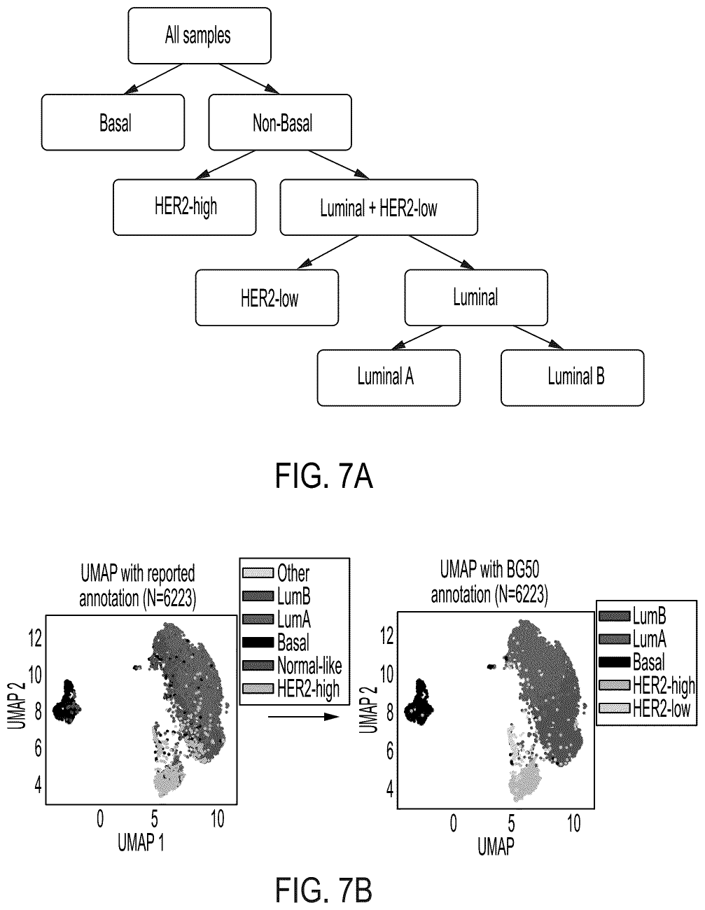

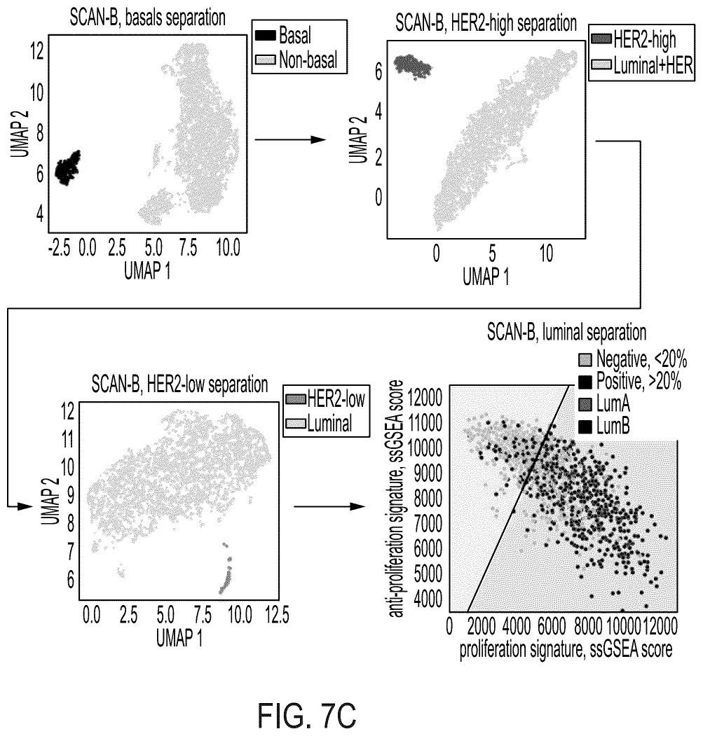

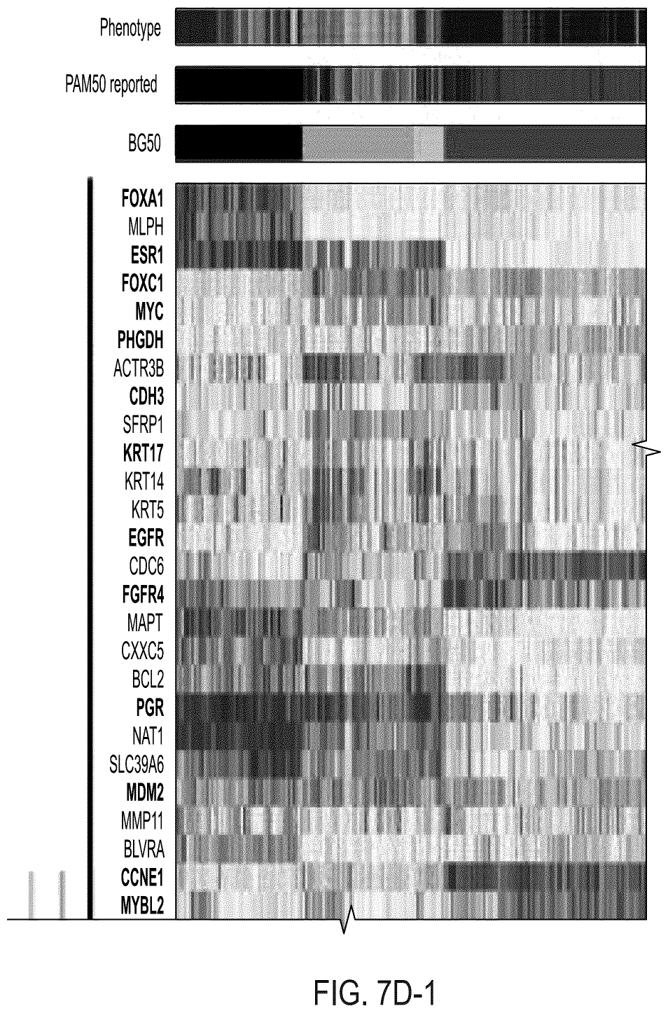

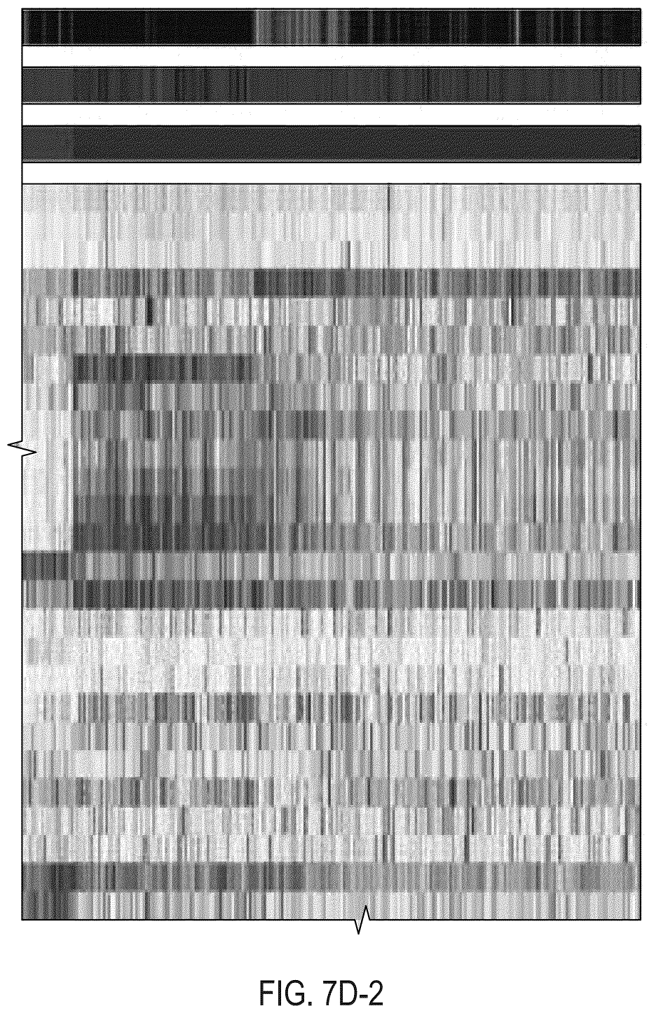

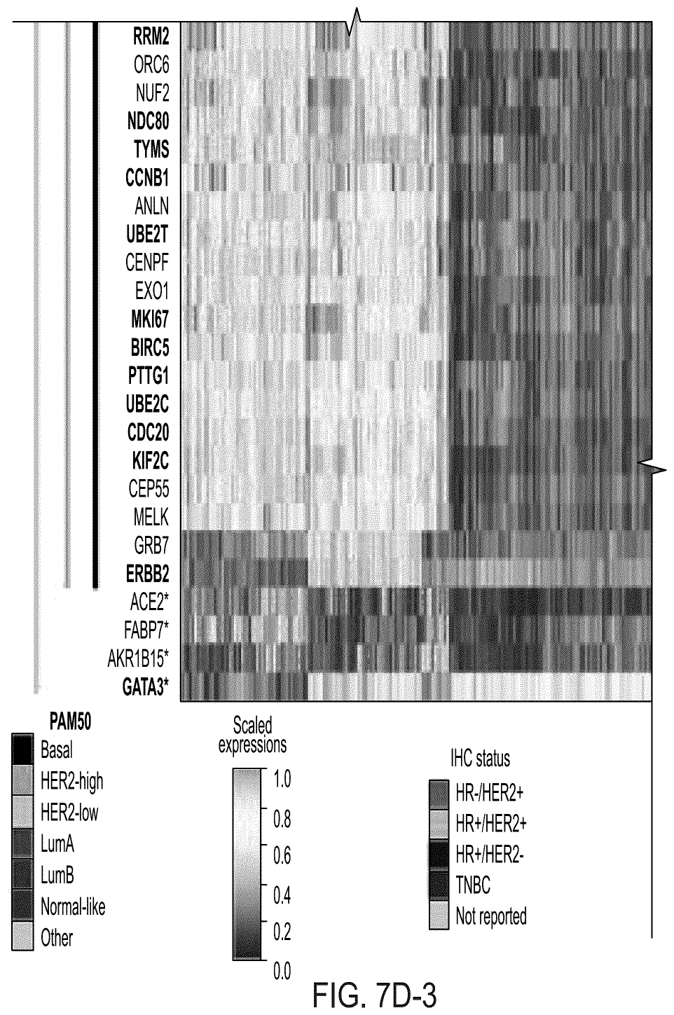

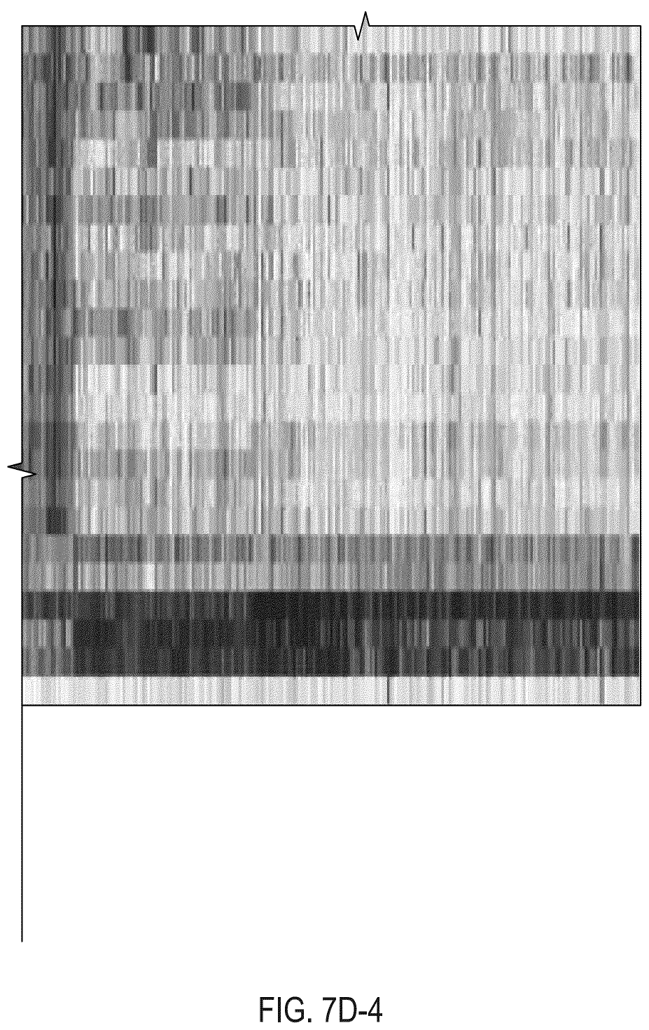

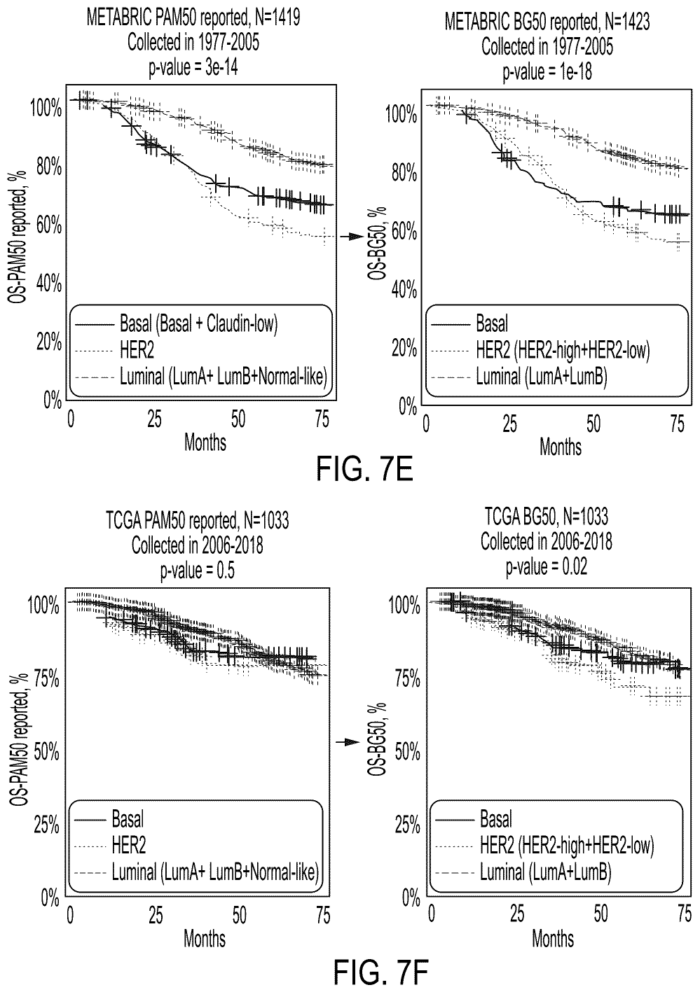

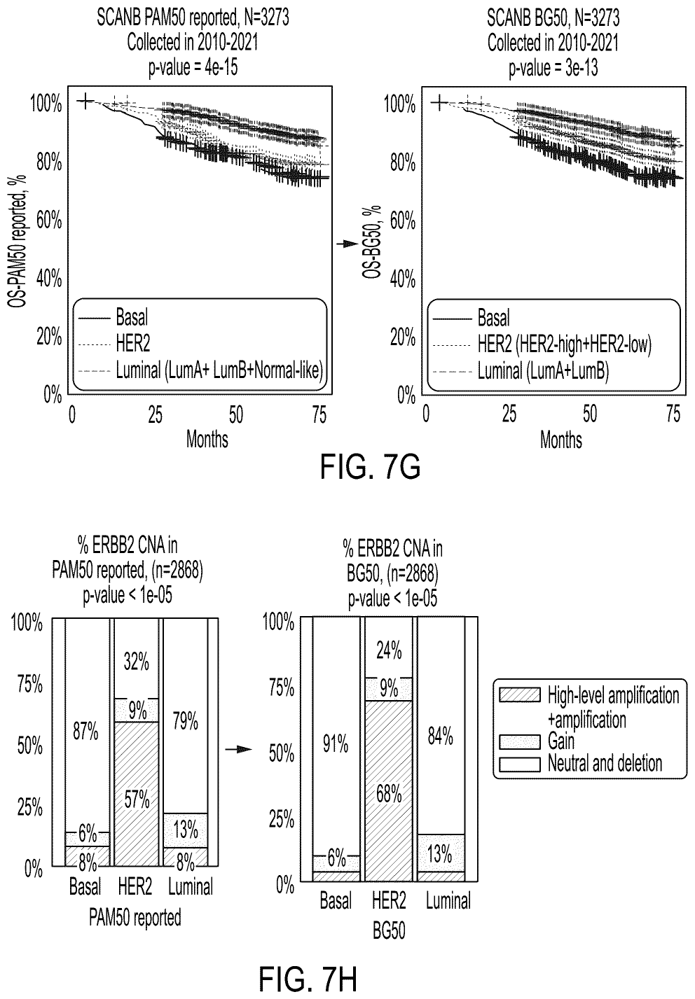

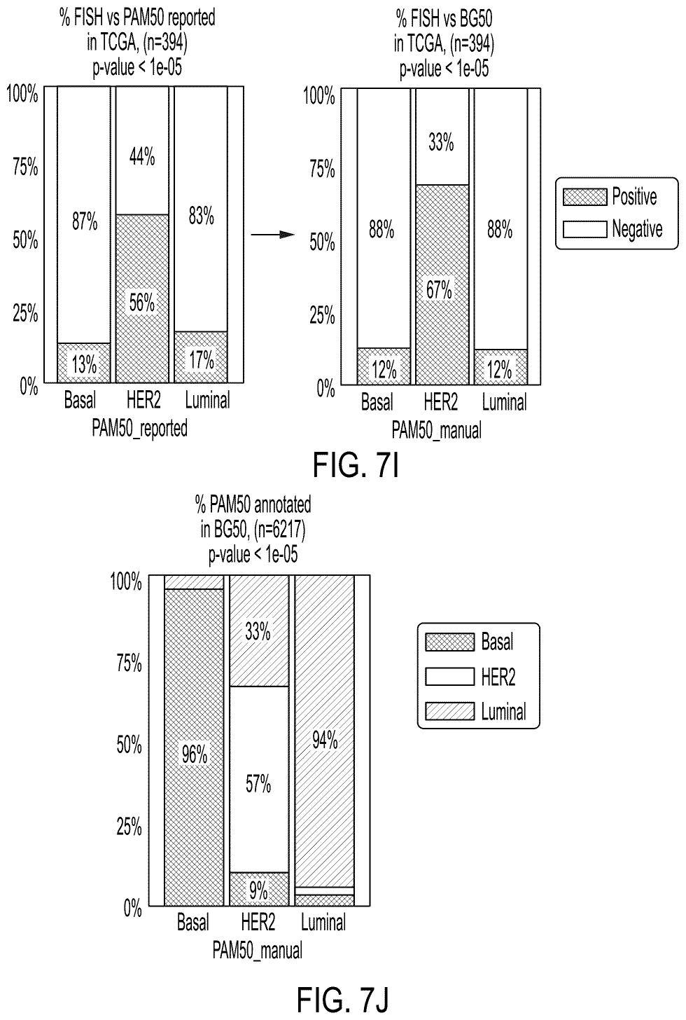

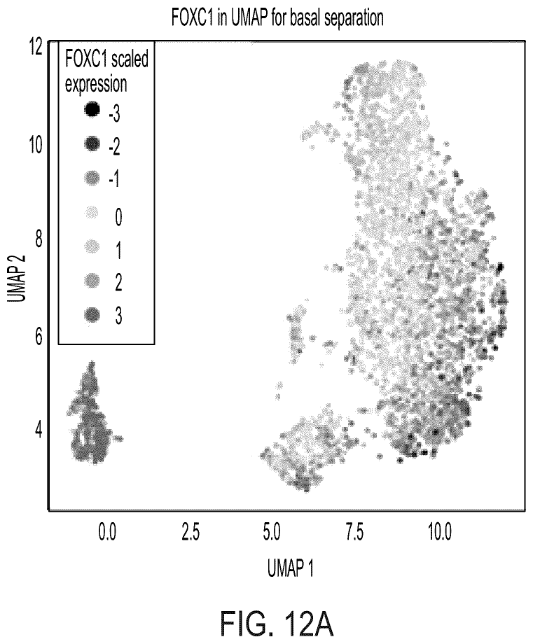

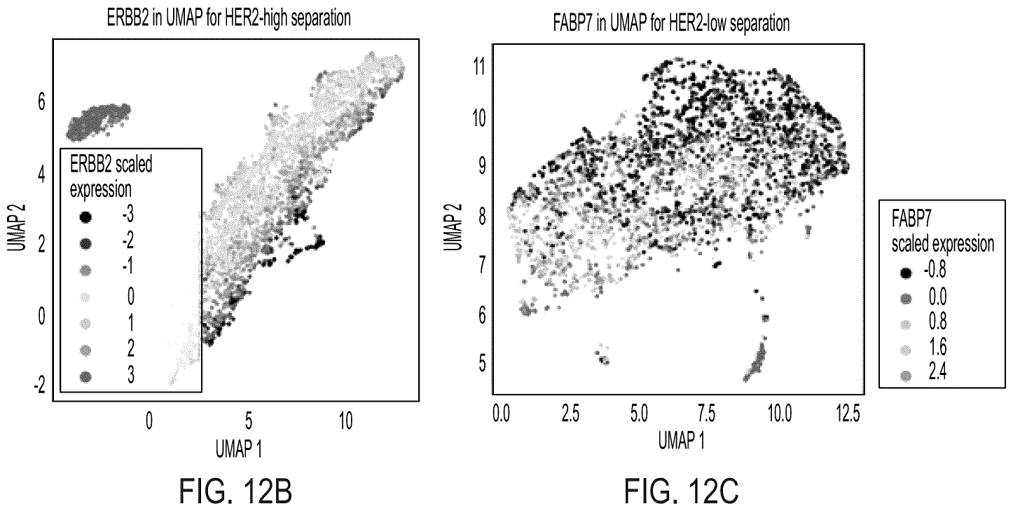

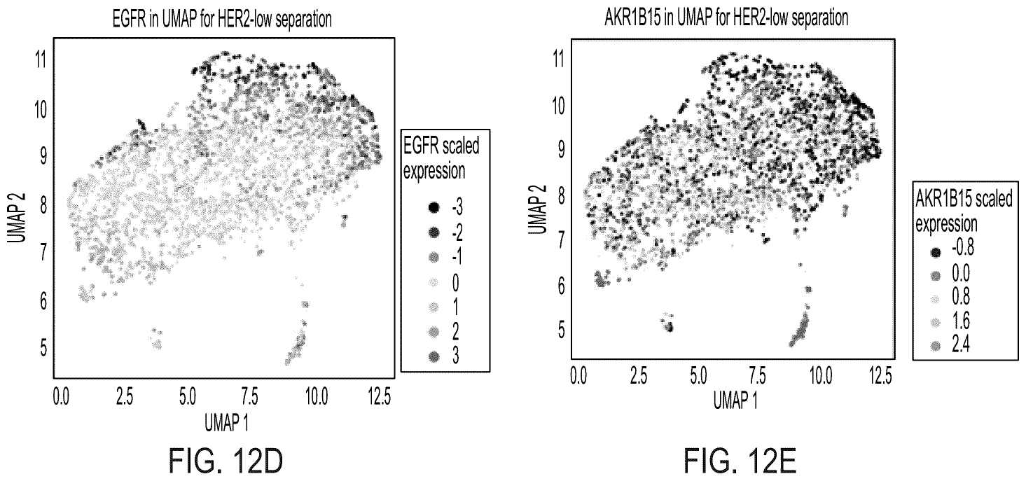

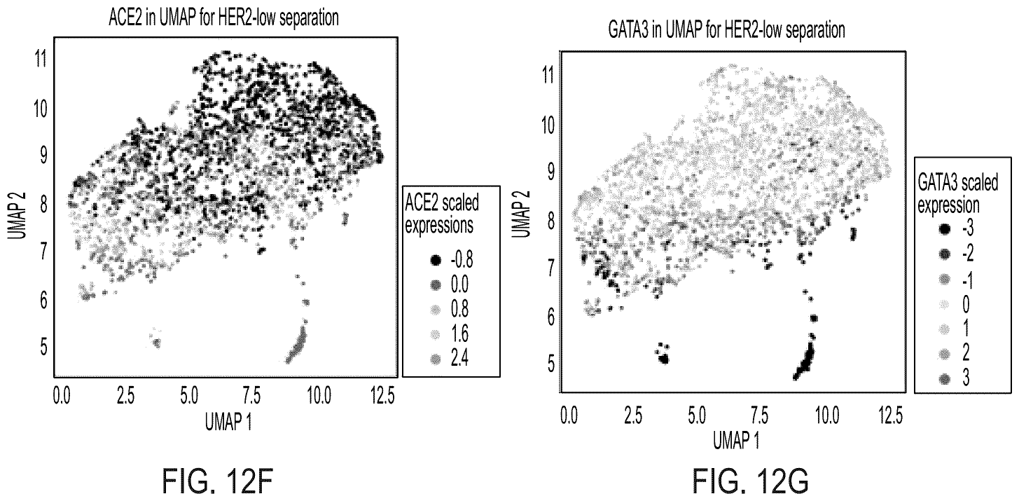

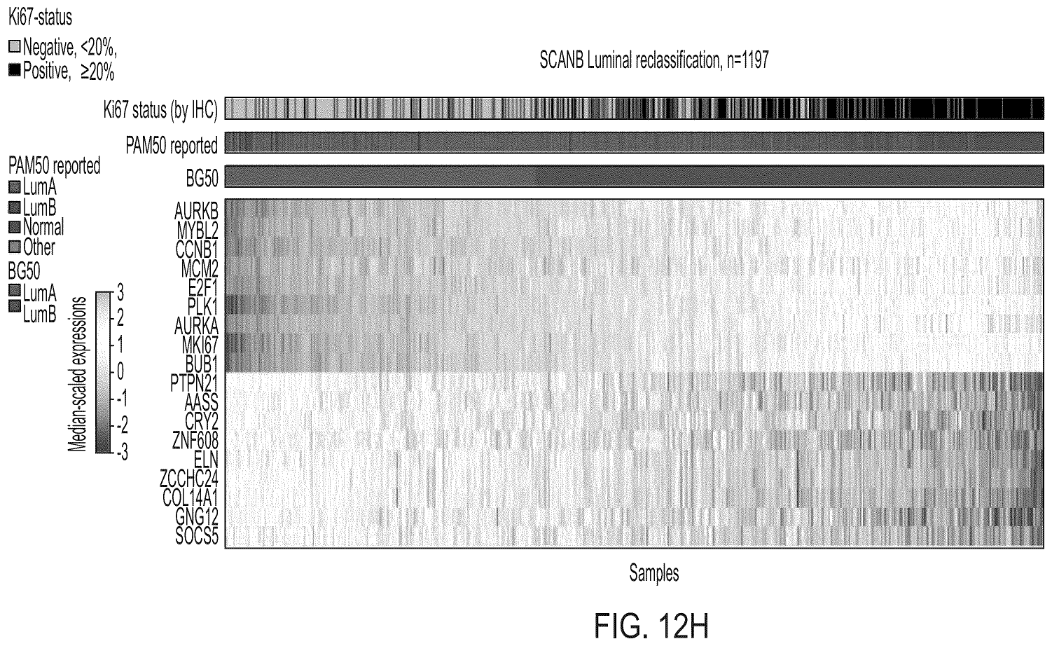

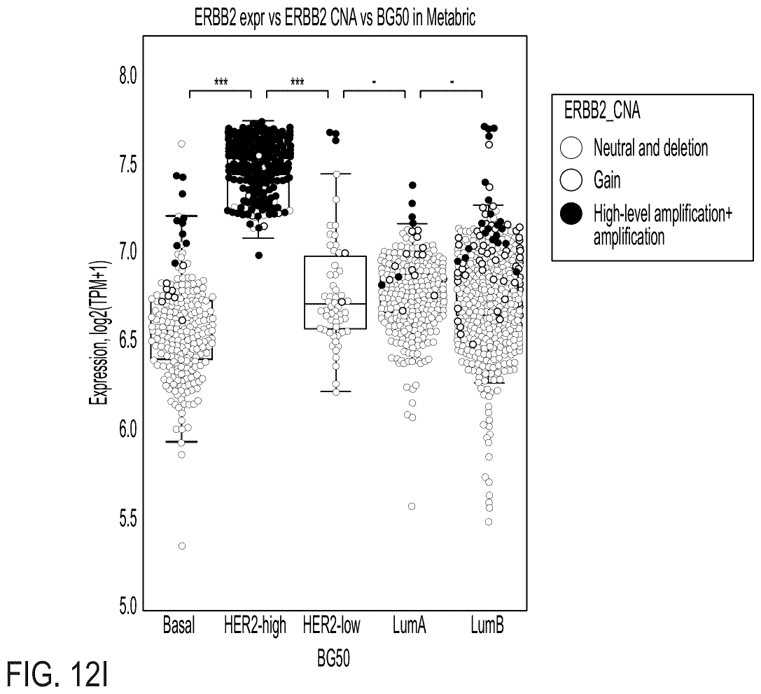

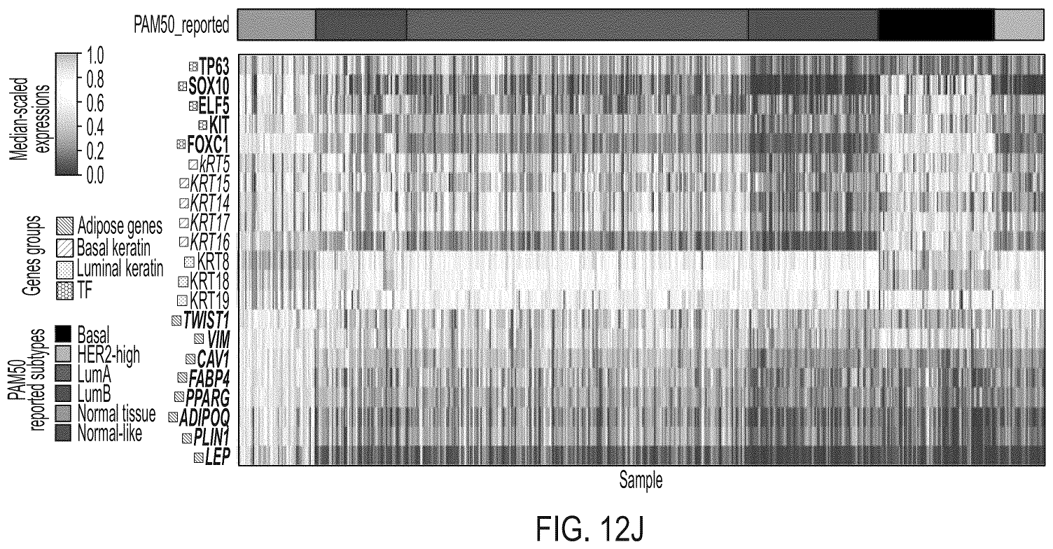

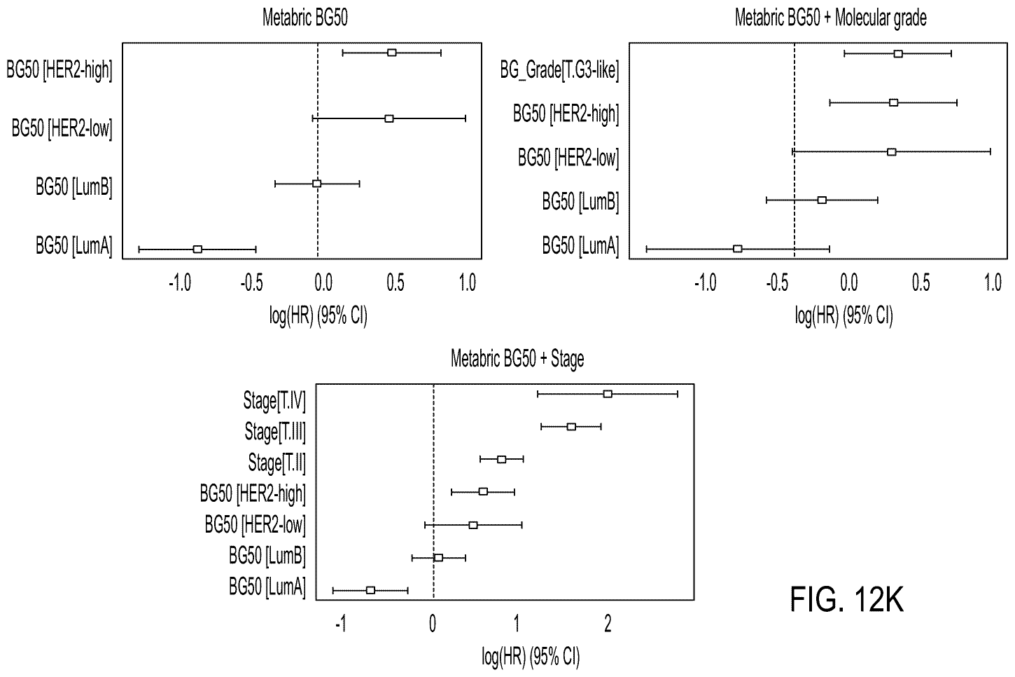

A new classification (referred to in some embodiments as “BG50”) was created using manual annotation which included a combination of clusterization techniques and implementation of signature scores. Clustering was performed separately for each dataset in different spaces of PAM50 genes and iteratively for each class (e.g. after one class of samples was defined, it was excluded and samples reclustered, then next class excluded and samples reclustered until all classes were assigned) (FIG. 7C). Firstly, preliminary clusterization using hierarchical clustering (distance measure metric was cityblock, linkage criteria was average) in classic PAM50 gene subset was done to define gene subsets which differ basals from other samples and HER2-high from other samples. It was observed that some samples with low ERBB2 expression were clustered together with HER2-high samples. They were extracted and differential expression between them and other samples was done. Four markers were identified as additional features for automatic separation (ACE2, FABP7, AKR1B15, GATA3). For exact class separation UMAP projection was done and then subtypes were divided in this projection using HDBSCAN clustering with different arguments. At first iteration UMAP projection was done in the space of all previously reported PAM50 genes and a group of samples which had high expression of FOXC1 gene (FIG. 12A) was confirmed as Basal subtype (Table 7). Next group was separated into two groups using a subset of proliferative genes (CCNE1, MYBL2, RRM2, ORC6, NUF2, NDC80, TYMS, CCNB1, ANLN, UBE2T, CENPF, EXO1, MKI67, BIRC5, PTTG1, UBE2C, CDC20, KIF2C, CEP55, MELK) and ERBB2 and GRB7. Separated samples which had high expression of ERBB2 gene (FIG. 12B) were assigned to HER2-high subtype.

Finally, samples were clustered again in the same subset of genes as on the previous step but additional transcriptional factors identified before were included (ACE2, FABP7, AKR1B15, GATA3). In this projection a small subset of samples was noticed and it was different from the rest group by expressions of FABP7, EGFR, AKR1B15, ACE2, GATA3 (FIGS. 12C-12G). This subset of samples was called HER2-low subtype. Further on the heatmap with all samples and all genes used for manual clusterization was identified that this new group has a similar profile of proliferative, basal and luminal genes as HER2-high group (FIG. 7D-1, FIG. 7D-2, FIG. 7D-3, and FIG. 7D-4). The rest of samples were classified as Luminal subtype and were subjected to further differentiation into Luminal A and Luminal B subtypes. In clinical practice, luminal samples (HR+ HER2− by IHC) are divided into subtypes according to their proliferation rate (doi.org/10.1038/A41572-019-011.1-2). A similar approach was applied in the current classification. Ki67-positivity (>=20% by IHC) is the key feature of Luminal B, a highly proliferative and aggressive subtype. Ki67-negative samples fall into Luminal A which is considered as a less aggressive subtype with better prognosis. A classifier was created to predict the Ki67-positivity of a sample based on the expression of two specific sets of genes: proliferative and anti-proliferative (FIG. 12H). These gene sets were defined by differential expression analysis of SCAN-B luminal samples, annotated by Ki67-status. This classifier was subsequently used to divide newly emerged Luminal group into subtypes Luminal A and Luminal B. This approach divides samples into 5 clusters (Basal, HER2-high, HER2-low, Luminal A, Luminal B). UMAP projections were done using umap package version 0.5.3 (McInnes, Leland, John Healy, and James Melville. “Umap: Uniform manifold approximation and projection for dimension reduction.” arXiv preprint arXiv:1802.03426 (2018)). For validation of annotation it was shortened to main subtypes: Basal, HER2 (HER2-high and HER2-low), Luminal (Luminal A+Luminal B). Reported annotation was also shortened to this subtypes (Normal likes, Luminal A and Luminal B were merged into Luminal subtype, HER2 and Basal subtypes remained unchanged).

TABLE 7
List of genes used for manual annotation of each subtype and for classifier training.

| | Basal | HER2-high | HER2-low | Luminal A + Luminal B |
|---|---|---|---|---|
| Genes for | FOXA1, MLPH, ESR1, | CCNE1, MYBL2, RRM2, | CCNE1, MYBL2, RRM2, | Proliferative signature: |
| manual | FOXC1, MYC, PHGDH, | ORC6, NUF2, NDC80, | ORC6, NUF2, NDC80, | CCNB1, AURKB, AURKA, |
| annotation | ACTR3B, CDH3, SFRP1, | TYMS, CCNB1, ANLN, | TYMS, CCNB1, ANLN, | PLK1, MCM2, BUB1, |
| | KRT17, KRT14, KRT5, | UBE2T, CENPF, EXO1, | UBE2T, CENPF, EXO1, | E2F1, MKI67, MYBL2 |
| | EGFR, CDC6, FGFR4, | MKI67, BIRC5, PTTG1, | MKI67, BIRC5, PTTG1, | Anti-proliferative signature: |
| | MAPT, CXXC5, BCL2, | UBE2C, CDC20, KIF2C, | UBE2C, CDC20, KIF2C, | GNG12, SOCS5, CRY2, |
| | PGR, NAT1, SLC39A6, | CEP55, MELK, GRB7, | CEP55, MELK, GRB7, | ELN, PTPN21, COL14A1, |
| | MDM2, MMP11, BLVRA, | ERBB2 | ERBB2, ACE2, FABP7, | ZNF608, ZCCHC24, AASS |
| | CCNE1, MYBL2, RRM2, | | AKR1B15, GATA3 | |
| | ORC6, NUF2, NDC80, | | | |
| | TYMS, CCNB1, ANLN, | | | |
| | UBE2T, CENPF, EXO1, | | | |
| | MKI67, BIRC5, PTTG1, | | | |
| | UBE2C, CDC20, KIF2C, | | | |
| | CEP55, MELK, GRB7, | | | |
| | ERBB2 | | | |
| Genes for binary | CCNE1, ANLN, TWIST1, | ACTR3B, LBP, CCNE1, | ACTR3B, KIT, CAV1, | Proliferative signature: |
| hierarchical | NUF2, ERBB2, MAPT, | BAG1, BCL2, ANLN, | LBP, KRT14, CCNE1, | CCNB1, AURKB, AURKA, |
| classifier | MLPH, SOX10, PHGDH, | NUF2, ERBB2, CDH3, | FABP4, BAG1, BCL2, | PLK1, MCM2, BUB1, |
| | AR, GPR160, ESR1, | MAPT, MLPH, PPARG, | ANLN, TWIST1, FGFR4, | E2F1, MKI67, MYBL2 |
| | TFAP2B, CDC6, GRB7, | MDM2, MKI67, GPR160, | ERBB2, MAPT, MLPH, | Anti-proliferative signature: |
| | ELF5, CDC20, FOXA1, | ESR1, FABP7, WNK4, | BLVRA, MDM2, MKI67, | GNG12, SOCS5, CRY2, |
| | FOXC1, PGR | CDC6, VIM, GRB7, | SOX10, PHGDH, AR, | ELN, PTPN21, COL14A1, |
| | | UBE2T, MMP11, CDC20, | GPR160, ESR1, FABP7, | ZNF608, ZCCHC24, AASS |
| | | KRT5, CTNNA1, KRT17 | TFAP2B, WNK4, CDC6, | |
| | | | CLDN8, GRB7, ELF5, | |
| | | | EGFR, MMP11, CDC20, | |
| | | | FOXA1, MYC, KRT5, | |
| | | | PGR, CCNB1, UBE2C | |

### Selection of Features for the Subtypes Description

To describe each subtype from a different molecular perspective (mutations, expressions, copy number alterations) features for were selected for the description. High and low expressed genes for each subtype were selected using differential expression. But before differential expression analysis genes with highest standard deviation were selected. Differential expression was done on two datasets (Metabric and SCAN-B), for each dataset 20 genes with top fold changes between subtypes were taken and intersected. Additionally transcriptional factors with high expression for each subtype were identified. All genes from those steps were further merged in one list and this list was refined by removal of those genes which were not mentioned in literature as biomarkers in breast cancer. Genes which have specific mutations for each subtype were selected using a chi-squared test. For each gene ratio between the number of patients with mutated and wild-type variants was calculated across all 5 subtypes. It was possible that the ratios were different between subtypes which was tested using chi-squared test with FDR correction. After that only significant genes were taken and those who had a ratio between the number of patients with mutated and wild-type variants more than 5% in at least one subtype were used for the description of subtypes. Genes with copy number alterations were selected from literature as those which were mentioned as biomarkers in breast cancer.

For the comparison of HER2-low subtype with other subtypes again differential expression was used. HER2-low was compared with Basal, HER2-high, Luminal (Luminal A+LumB) subtypes subsequently on TCGA dataset.

### BG50 Classifier Training

Manual subtype annotation performed on SCAN-B and TCGA datasets was subsequently used to train a machine-learning (ML)-based hierarchical classifier. At each step, a separate classifier makes a binary prediction, assigning the test sample to one of two groups. Architecture of the classifier is the same as in the manual annotation approach. Dead end groups are assigned to molecular subtypes; composite groups are subjected to separation by the subsequent classifier in the next step.

Basal, HER2-high and HER2-low subtypes are distinguished at steps 1, 2, 3 respectively (FIG. 7A). These steps are performed by LightGBM-classifiers that process the expression of specific genes (features) in ranks. Updated PAM50 genes list (DOI: 10.1038/35021093) with transcriptional factors mentioned in annotation part was taken as initial features for each LightGBM-classifier, then the recursive feature elimination algorithm where importance was evaluated using shap tool (10.48550/arXiv.1705.07874) determined the optimal sets of features (Table 7). Thereafter, the classifiers were trained and validated on subsets from the combined SCAN-B+TCGA dataset. F1 scores were 0.99, 0.95 and 0.82 for Basal, HER2-high and HER2-low subtype, respectively. Next step 4 which divides the Luminal group into Luminal A and Luminal B subtypes is arranged differently. Differential expression analysis by DESeq2 algorithm (10.1186/s13059-014-0550-8) was utilized to identify the most up- and down-expressed gene sets in SCAN-B Luminal samples, annotated as Ki67+ (>=20% by IHC). Enrichment scores (ES) (Subramanian et al. (2005)) for each of these two sets of genes were calculated and used as explanatory variables to train a logistic regression classifier. Thus, taking as input ES of two specific gene sets, the classifier predicted Ki67-positivity of a luminal breast cancer sample. Quality of prediction was validated on GSE21653 dataset, with F1 score 0.77. Luminal samples defined by the regression as Ki67-positive fall into the Luminal B subtype; samples defined as Ki67-negative into the Luminal A.

The hierarchical classifier was capable of making single-sample predictions, as it did not utilize per-cohort scaling. The possible technical batch effect was reduced by using ranking and ES approaches, thus the classifier could be used on gene expression data from various platforms. The main limiting factor was the availability of a sufficient set of features for each classifier: in the absence of some genes, the performance may be worse.

### Histological Examination

### Survival Analysis

Survival differences were assessed using log rank tests from lifelines version 0.27.3 (Davidson-Pilon, (2019). lifelines: survival analysis in Python. Journal of Open Source Software, 4(40), 1317, https://doi.org/10.21105/joss.01317) and presented on Kaplan-Meiers plots. For survival analysis amongst biomarkers, single-variate Cox regression modeling was conducted controlling BG50 annotation, molecular grades and stages.

### IHC Labels by RNA-Seq Cut-Offs

The alternative approach to define IHC status using RNA-seq data is to compare the ESR1 and ERBB2 gene expressions with selected cut-offs (V. Kushnarev et al. PATHOBIOLOGY AND EMERGING TECHNIQUES. Laboratory Investigation. 2023; 103(3):S1551, https://doi.org/10.1016/j.labinv.2023.100098) Samples from the TCGA dataset were labeled using this approach (FIG. 13C). Expressions were log 2-transformed, and the selected cutoffs were 3.5 for ESR1 and 8.5/6 for ERBB2. Thus, the classification was:

TABLE 8

| Subtypes by | ESR1 expression, | ERBB2 expression, |
|---|---|---|
| RNA-seq cutoffs | log2(TPM + 1) | log2(TPM + 1) |
| HER2+/ER− | ESR1 < 3.5 | ERBB2 ≥ 8.5 |
| HER2+/ER+ | ESR1 ≥ 3.5 | ERBB2 ≥ 8.5 |
| HER2-low | any | 6 ≤ ERBB2 < 8.5 |
| HER2−/ER+ | ESR1 ≥ 3.5 | ERBB2 < 6 |
| TNBC | ESR1 < 3.5 | ERBB2 < 6 |

### Results

A total of 6223 breast tumor samples from public METABRIC, TCGA, and SCAN-B cohorts were utilized to define intrinsic subtypes based on rank expression of PAM50 genes. The refined classification better distinguishes Basal and HER2-enriched samples from Luminal and Normal-like subtypes. Suggested reclassification allows better UMAP separation of assigned subgroups than it was in previously reported annotation (FIG. 7B).

### Validation of Main Subtypes

The clinical connotation of 6223 samples, which were divided into three main subtypes: Basal, HER2-enriched, and Luminal (further divided into Luminal A and Luminal B) were analyzed. These three main subtypes showed similarity to the iconic PAM50: 96% of samples from the Basal subtype in the new annotation match with Basal samples from the original classification. Likewise, for the HER2-enriched subtype, this percentage of concordance with previously published annotation is 56%, and 94% for Luminal. BG50 (the classifier) demonstrates the enrichment of HER2-enriched cases notable to the original classification (FIG. 7J). As part of the validation, Kaplan-Meier plots of the initially published classification and the classification were compared. For the Metabric dataset, reported classification 5-year survival was 68, 58, 82% for Basal, HER2-enriched, and Luminal and 66, 59, 83% for the new classification respectively (FIG. 7E). For the TCGA cohort (FIG. 7F), the difference in overall survival between subtypes is significant for the classification (p-value=0.02) compared with initially published PAM50 classification (p-value 0.5). Similarly, for SCAN-B (FIG. 7G) cohort, 5-year survival for Basal is 78%, for HER2-enriched 81%, and for Luminal subtypes is 90% for initially published classification. The classification showed high concordance with these results and 80-month survival was 77, 84, 90% for Basal, HER2-enriched, and Luminal respectively.

Moreover, the reclassified HER2-enriched subtype showed better concordance with HER2 positivity identified by IHC data from TCGA. The number of cases with ERBB2 amplifications in HER2-enriched subtype was increased from 58% to 68% in the new annotation of TCGA and Metabric datasets (FIG. 7H). Finally, the ratio between FISH-positive and FISH-negative patients across main PAM50 subtypes in the iconic and new classification was compared and (FIG. 7I) the number of FISH-positive cases in the HER2-enriched subtype was increased (from 56% to 67%) in the new annotation.

### Detailed Characterization of Subtypes

During the investigation of genetic features of 5 new molecular BRCA subtypes, some patterns of alterations across several groups of genes were revealed. Most oncogenic suppressor genes such as TP53, BRCA1, PTEN, CDH1, RB1, ARID1A, MAP3K1, FAT3, KMT2C, KMT2D, FBXW7, CREBBP, MAP2K4 possess inactivation mutations or deletions. Moreover, in well-known oncogenes like PIK3CA, ERBB2, AKT1, GATA3, SF3B1, CCNE2, FGFR1, TOP2A, EGFR, FGFR2, MET amplification and likely activation mutations were identified (FIG. 8B).

### Basal

From the prospect of molecular and clinical features, Basal BG50 subtype is quite similar to the TNBC IHC phenotype (which is 82.3% of the Basal subtype, FIG. 8A).

In terms of 5-year survival rates, Basal subtype (along with HER2-low) had the worst survivability (77%) in the SCANB cohort (collected in 2010-2021, FIG. 5C, lower right plot). In Metabric cohort (collected in 1977-2005, before HER2-target therapy started to apply, upper plot) Basal subtype survival rate was 66%, which was lower than Luminal A and Luminal Bsubtypes but higher than HER2-high and HER2-low subtypes.

Coding mutation rate of TP53 in Basal subtype was the highest among subtypes (83%, pval<1e-108). BRCA1 germline mutation was also a relatively common event for basal samples (10%, pval<1e-8) along with BRCA1 somatic mutation (6%, pval<1e-4). Unlike other subtypes, in Basal subtype amplifications in GATA3 (22%, pval<1e-46) and PIK3CA (9%, pval<1e-11) were more often met.

Despite these results PIK3CA of GATA3 mutations occur more less in basal subtype then in luminal subtypes. Deep deletions of PTEN were detected in 6% of basal samples, and this event can also be considered more characteristic of the basal subtype (pval<1e-5).

Compared to each of the other subtypes, Basal subtype showed high expression of basal phenotype marker genes FOXC1 and KRT11; low expression of luminal marker genes FOXA1 and GATA3 (doi.org/10.1186/bcr2327). Expression of hormone receptor genes (AR, ESR1, PGR) was the lowest in basal subtype, this also correlates with a high percentage of TNBC samples in this subtype (82%). Basal subtype can also be characterized by high expression of cell cycle (MYC, MKI67, CCNE1, CDK1, CCNB1, MYBL2, CCNB2, E2F2, CDC20) and mitosis (PTTG1, AURKB, NDC80, KIF2C, TPX2, TOP2A) gene signatures. Basal samples have the lowest expression of kinase receptor genes ERBB2 and ERBB3 and second highest (after HER2-low) expression of EGFR. Of other features, Basal subtype has the highest expression of differentiation marker gene KRT17; highest expression of genes CDH3(doi.org/10.21873/anticanres.14568), UBE2C, UBE2T, TOP2A, BIRC5, PHGDH and TYMS; and the lowest expression of AGR2, XBP1, TFF1 and CA12 (FIG. 8E). Basal is the leading subtype in terms of JAK-STAT, MAPK, NFkB, PI3K and TNFa signaling pathways, while p53 and androgen pathways have the lowest signaling in Basal.

According to the deconvolution data, Basal subtype has increased number of NK-cells (higher than in HER2-high, Luminal A and Luminal Bsubtypes; pval<0.001); B-cells (higher than in Luminal A and Luminal Bsubtypes; pval<0.001) and CD4+ T-cells (higher than in Luminal A and Luminal Bsubtypes; pval<0.001). The number of fibroblasts is reduced in the basal subtype (lower than in HER2-high, Luminal A and Luminal Bsubtypes; pval<0.001) (FIG. 13A).

HER2-high

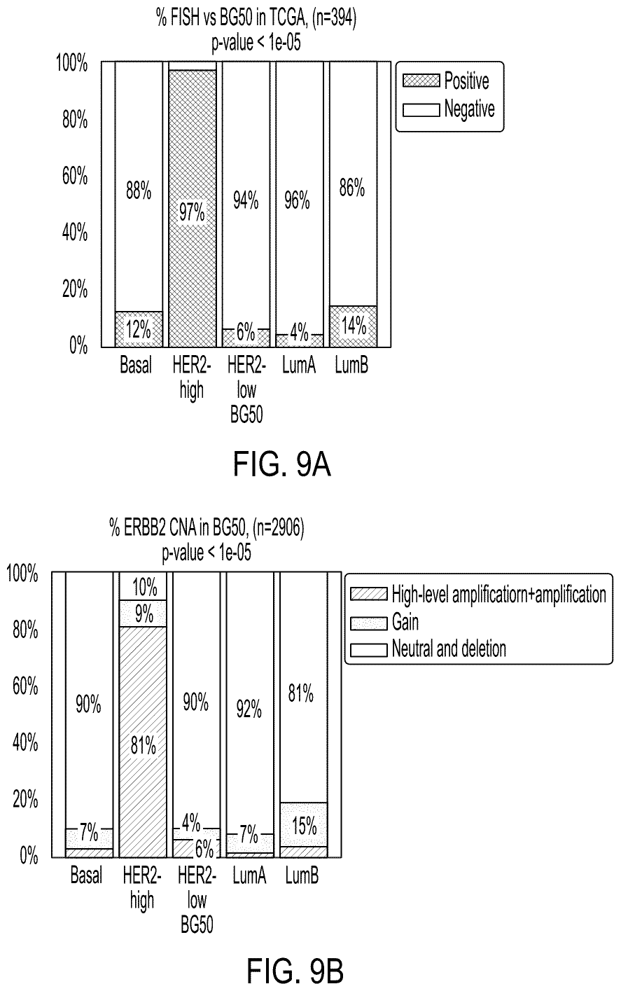

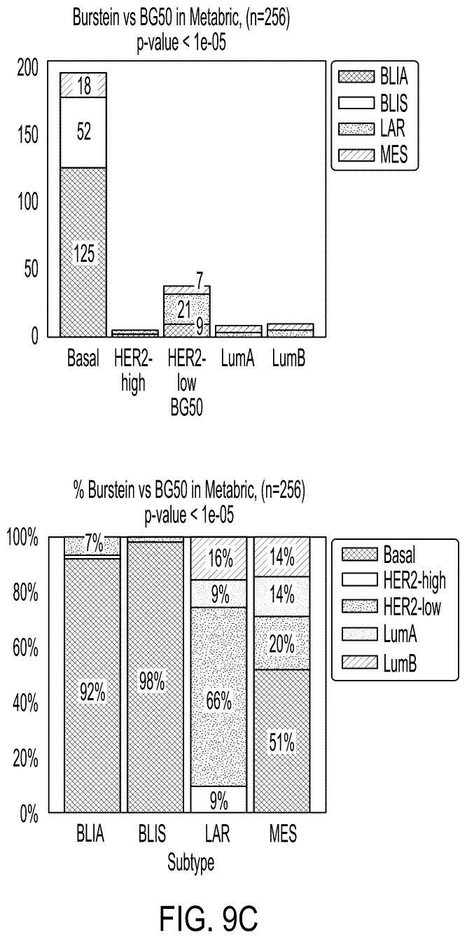

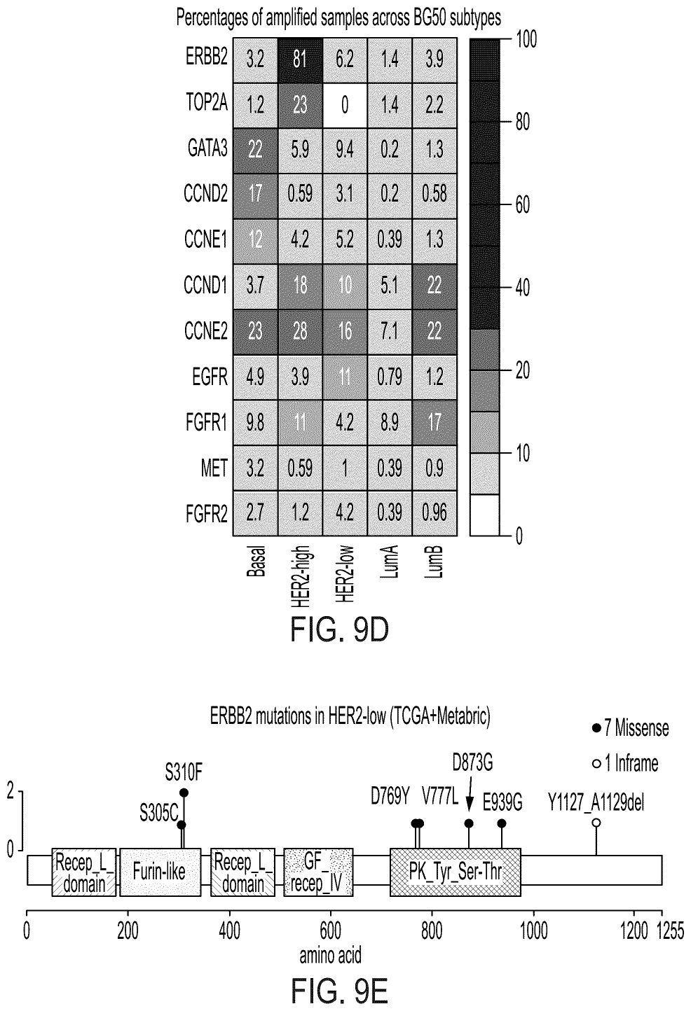

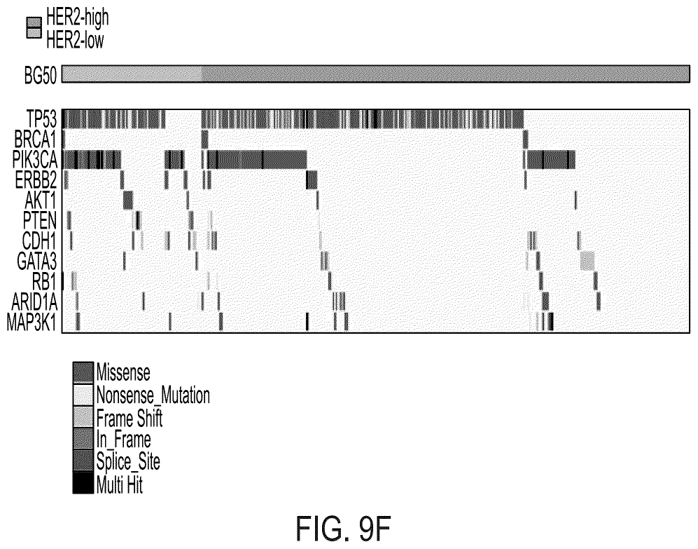

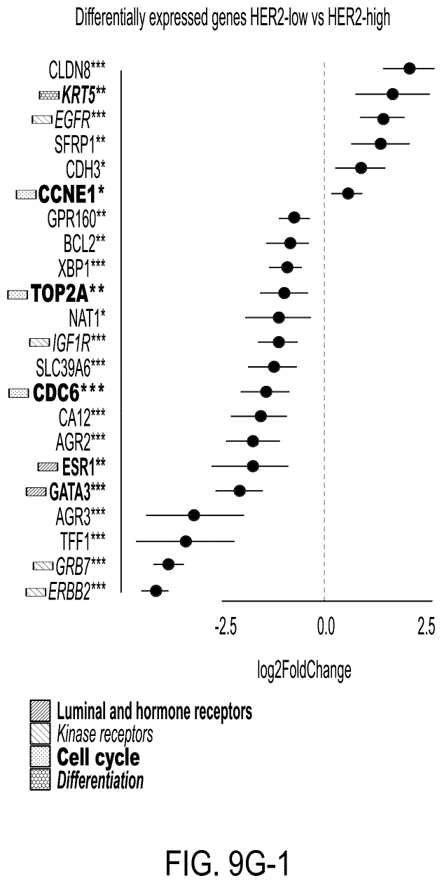

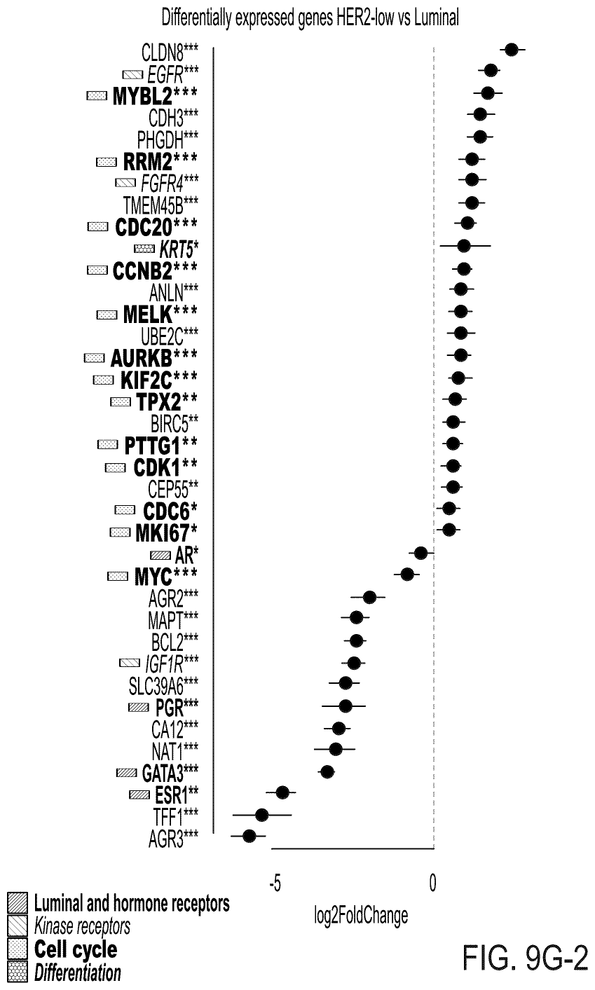

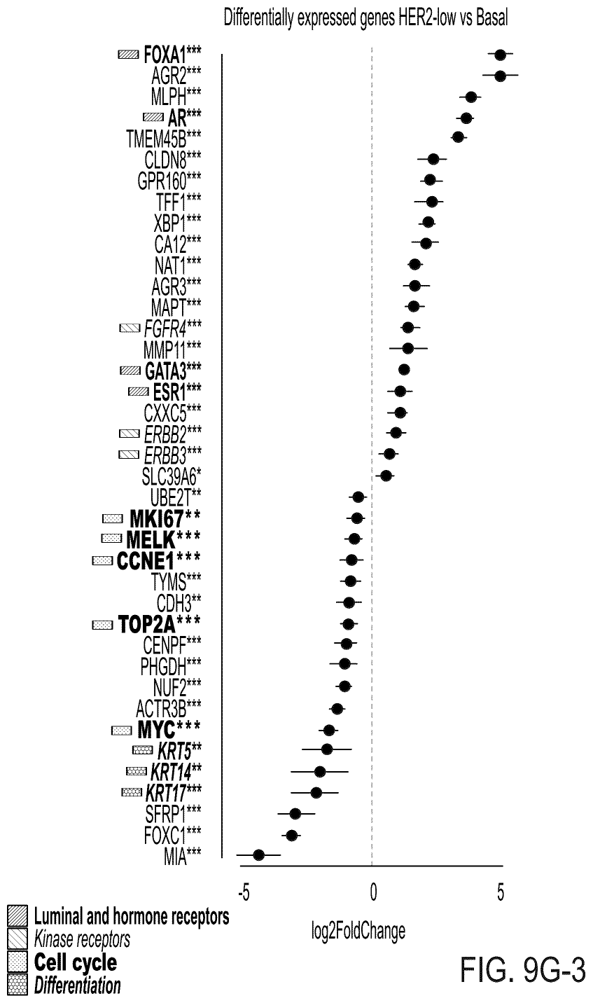

The HER2-high subtype demonstrates common expression, mutational, and amplification features specific to its subtype. 91% of samples from this subtype show HER2 positivity by IHC according to TCGA, Metabrick, SCAN-B annotation (FIG. 8A). 5-years survival rate for this subtype in Metabric dataset was 60%, which is lower than in Basal, Luminal A and Luminal B subtypes. However in SCAN-B dataset (collected after Trastuzumab approval 10.1200/JCO.2002.20.3.719 and DOI: 10.1056/NEJMoa052306) 5-years HER2-high subgroup survival was 86%, which is higher than the Basal and HER2-low subtype (FIG. 5C). Genomic profile of this subtype shows similar mutational events as it was previously reported in literature [doi: 10.15252/emmm.202012118]. 79% of samples from this subtype show amplifications in ERBB2 (FIG. 8C). Moreover, this subtype shows 97% concordance with ERBB2 amplification by FISH method, reported in TCGA (FIG. 9A). There was a significantly high percentage of coding mutations in TP53 gene in comparison with other subtypes except Basal (65% of mutated samples, p-value=2e-34, FIG. 8C). Further expression profile was investigated using the same statistical tests described in the previous part (Basal description). Compared to Basal, HER2-low and Luminal subtypes, HER2-high subtype showed highest expression of ERBB2. Expression of ERBB3 gene in HER2-high is higher than in HER2-low subtype. HER2-high subtype can also be characterized by high expression of cell cycle (MKI67, CDK1, CCNB1) genes, mitosis genes (TPX2, AURKB, NDC80) and such important markers as UBE2C, UBE2T, TOP2A in comparison with HER2-low and Luminal subtypes (FIG. 8E). Moreover, ESR1 gene in this subtype has bimodal distribution which is explained by heterogeneity of hormone status by IHC in this subtype (34% of samples are HR−/HER2+ and 57% are HR+/HER2+, FIG. 8A). Finally, level of progeny pathways in HER2-high subtype was compared with other subtypes. It was revealed that EGFR pathway have statistically significant high level of scores in HER2-high. Deconvolution profile of this subtype showed low level of Naive T-cells and Naive CD8 T-cells (lower than in Basal, Luminal A, LumB) (FIG. 13A).

### HER2-low

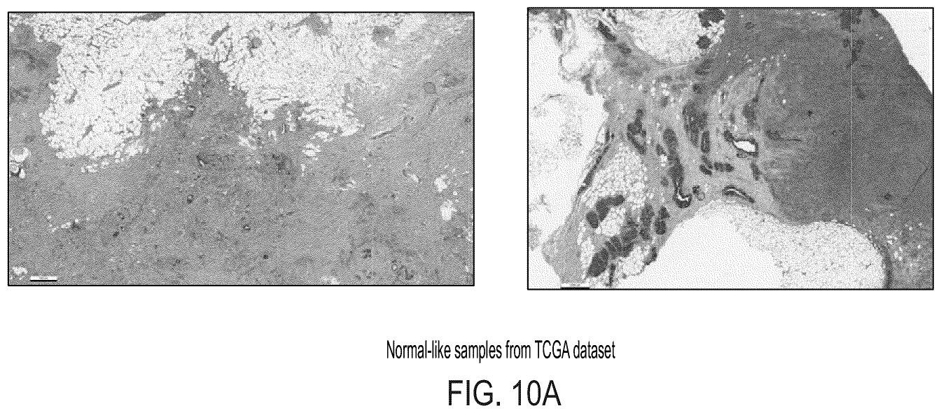

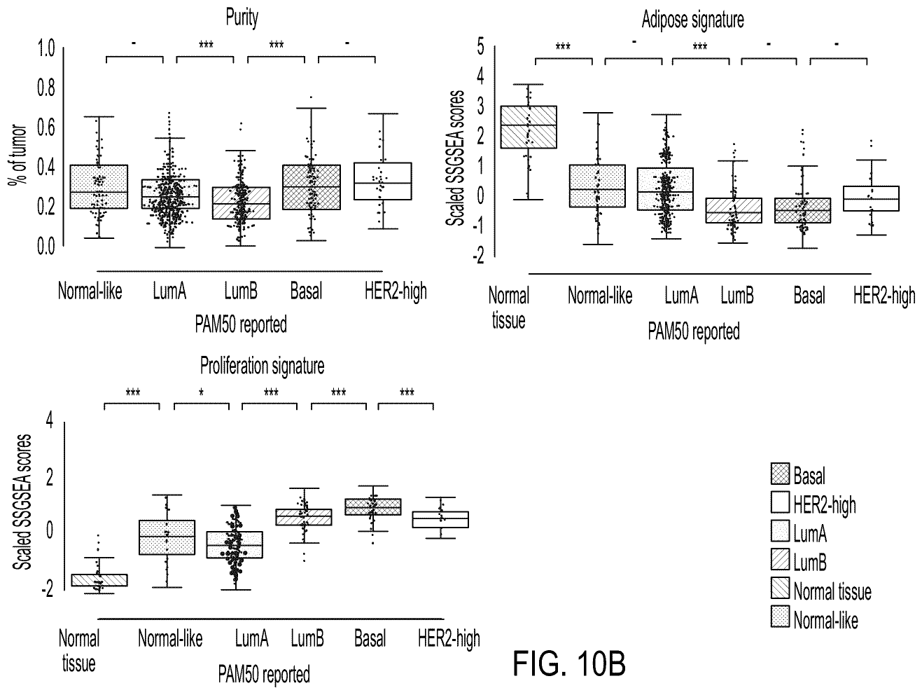

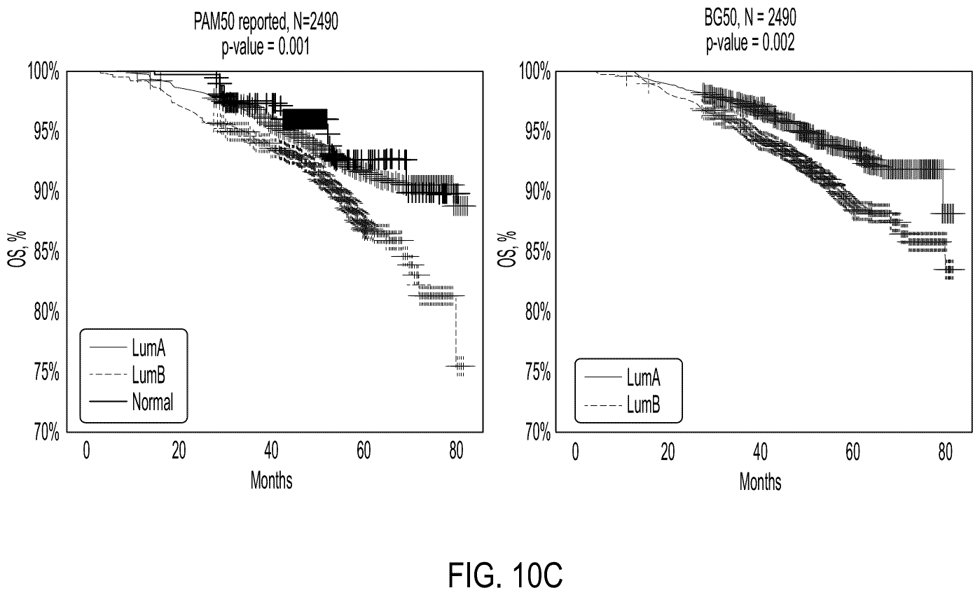

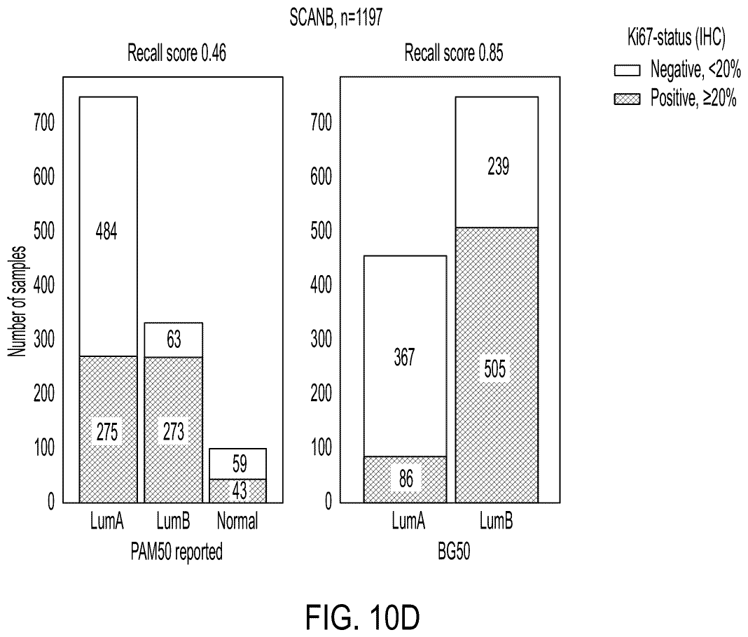

HER2-low subtype was recently considered as an IHC biomarker, but in this example it was identified as a separate molecular group with unique biology, drivers and characteristics features. 69% of patients from this subtype have TNBC IHC subtype and only 6% of the samples has HER2-positivity by IHC (FIG. 8A). The HER2-low subtype is enriched with the LAR class when compared to the Burstein classification of triple-negative breast cancer (TNBC) samples (55% of HER2-low samples belong to LAR subtype) (FIG. 9C). 5-years survival of HER2-low in the Metabric dataset was similar to HER2-high (55% and 60% respectively) and was the worst among all subtypes. 10-years survival of HER2-low in Metabric shows the same pattern: still similar to HER2-high (50% and 45%), 15-years survival of HER2-low is the worst among all other subtypes (40%). 5-years survival of HER2-low in the SCAN-B dataset was similar to Basal subtype (76% and 77% respectively) and again was the worst among all subtypes (FIG. 5C). Only 6% of the samples had HER2-amplifications measured by FISH method (FIG. 9A) and 10% of samples had amplifications (FIG. 9B) which proves that this subtype is not similar to HER2-high group.

The HER2-low subtype in IHC classification is characterized by 1+ or 2+ scores without amplifications (by FISH-method). The molecular HER2-low subtype is concordant with this definition. 92% of patients from this subtype have ERBB2 expression between 6 and 8.5 log 2 TPM which was previously defined as a cut-off for IHC HER2-low concordance (FIG. 6B) [(V. Kushnarev et al. PATHOBIOLOGY AND EMERGING TECHNIQUES. Laboratory Investigation. 2023; 103(3):S1551. doi:10.1016/j.labinv.2023.100098)] and only 6% of then have ERBB2 amplifications (FIG. 9D), which means that 84% of HER2-low group (2.3% of all cohort) satisfy requirements for the HER2-low IHC group. At the same time all samples from HER2-high group have ERBB2 expression higher than 8.5 log 2 TMP and 87% of samples have amplifications in ERBB2 gene which shows consistency with HER2-enriched IHC subtype (FIG. 6B). Same distribution of expression of ERBB2 amplified samples can be seen in Metabric dataset (FIG. 12I). However, HER2-low IHC group is not the same as HER2-low molecular group because into IHC group can be included 46% of Basal samples, 97% of Luminal A and 86% of Luminal B according to RNA-seq cut-offs (FIG. 13C). RNA-seq cut-offs for IHC are considered as more precise because they are not affected by the factor of human error. 100% of HER2-low samples are nor HER2-positive by this method (FIG. 13B).

Deep investigation of CNA statistics showed that HER2-low subtype has highest percentage of samples with amplifications of EGFR gene (11%, p-value=6e-5, FIG. 9C) among other subtypes. Also, HER2-low subtype can be characterized with high percentage of amplifications in AKT1 (9%, p-value=1e-5). There was a significantly high percentage of coding mutations in TP53 (72%, p-value=1e-13), PIK3CA (53%, p-value=0.02), ERBB2 (8%, p-value=0.05), KMT2D (5%, p-value=0.01), PTEN (11%, p-value=0.04) (FIG. 8C). Distribution of mutations in ERBB2 gene in HER2-low subtype was investigated closer because ERBB2 gene is considered as one of the main drivers in this subtype. It was revealed that mutations in this gene are mostly identified as missense and occur in the tyrosine kinase domain (FIG. 9E). Mutations in TP53 are mostly presented by missenses and frame shifts, in PIK3CA by missense and multi hit (FIG. 9F).

Further expression profile of the HER2-low subtype was investigated. The expression profile of HER2-low subtype is related to HER2-high samples but does not have high expression of ERBB2 and adjacent GRB7 genes distinguishing samples with HER2 amplification. Compared to Basal, HER2-high and Luminal subtypes, HER2-low subtype showed the highest level of EGFR and CLDN8 expression and lowest level of IGFR1 expression. Compared with HER2-high and Luminal subtypes, the HER2-low subtype has the lowest expression of cell migration marker AGR2, luminal markers GATA3 and FOXA1, hormone receptors ESR1, PGR, tyrosine kinase receptor ERBB3 and such important markers as CA12 10.1111/jcpt.13580, XBP1 10.1016/j.canlet.2020.05.020, TFF1 10.1016/j.bone.2020.115775. During investigation of Progeny pathways, it was revealed that HER2-low subtype has the highest level of Androgen, Hypoxia, p53 and relatively high level of NFkB, TNFa pathways. Moreover HER2-low showed the lowest level of Estrogen pathway score.

Deconvolution profile of this subtype showed high level of regulatory NK-cells, (significantly higher than in Basal and Luminal A, Luminal B subtypes); and low level of Naive T-cells and Naive CD8 T-cells than in Basal and Luminal A, Luminal B subtypes) (FIG. 13A).

Further uniqueness of the new HER2-low subtype was confirmed by differential expression analysis between other three groups (Basal, HER2-high, Luminal which is combined Luminal A+LumB).

### HER2-Low Gene Enrichment Pathway Analysis

Central to the HER2low subtype's identity is its distinctive relationship with cell cycle dynamics, underscored by differential gene expression that encompasses key processes such as Differentiation, Kinase Receptor Signaling, Cell Cycle Regulation, and Luminal Phenotype Characteristics (FIG. 9G-1, FIG. 9G-2, and FIG. 9G-3). This subtype is characterized by the overexpression of several genes crucial for cell cycle progression, such as MYBL2, RRM2, CDC20, MKI67, and BIRC5, particularly when juxtaposed against Luminal tumors. This upregulation hints at an enhanced cell proliferation potential, a feature further emphasized by CCNE1's overexpression, which implies an accelerated G1 to S phase transition. In contrast, this expression is notably subdued when compared to Basal subtypes, suggesting distinct proliferation dynamics across these subtypes. Additionally, the fluctuation of mitotic genes like MELK and TOP2A might impact mitotic integrity and dynamics, with BIRC5 (Survivin) overexpression indicating potential resistance to apoptosis-inducing therapies in the HER2low context.

The HER2low subtype straddles the line between basal and luminal phenotypes, exhibiting a complex basal-luminal identity. The overexpression of KRT5 points to basal features, contrasting with its reduced expression compared to Basal subtypes. Concurrently, FOXA1 overexpression suggests luminal characteristics. This mixed basal-luminal identity is further complicated by the expression patterns of hormonal signaling genes. The subtype shows lower levels of hormone-related genes such as ESR1, PGR, and GATA3, especially in comparison to Luminal tumors, indicating reduced estrogen and progesterone signaling. However, the expression of these markers increases when HER2low is compared to Basal tumors, including AR, hinting at a potential responsiveness to hormonal interventions.

Another aspect of HER2low tumors is the overexpression of EGFR, contrasting with both HER2high and Luminal tumors. This finding may indicate a reliance on alternative growth and survival pathways, potentially opening avenues for targeted EGFR therapies. Despite its nomenclature, the HER2low subtype interestingly shows increased ERBB2 expression compared to Basal tumors, underlining the complexity of its molecular landscape.

The genes involved in cell adhesion and migration, such as CLDN8, CDH3, SFRP1, MMP11, and TMEM45B, are overexpressed across various comparisons. This overexpression suggests alterations in cell-cell adhesion and potential changes in migratory or invasive properties, impacting tumor aggressiveness and metastatic capability.

Finally, the metabolic landscape of HER2low tumors is marked by the expression of genes like PHGDH, SLC39A6, NAT1, and CA12, which are downregulated compared to HER2high. This pattern reflects a nuanced interplay of amino acid synthesis, ion homeostasis, detoxification processes, and pH regulation, suggesting potential metabolic vulnerabilities within these tumors. Notably, the expression of these genes varies when compared to Luminal and Basal subtypes, underscoring the metabolic heterogeneity within HER2low tumors.

### Luminal in General

Classification of SCAN-B luminal samples into the Luminal A and Luminal B subtypes better predicted Ki-67 positivity (≥20% of Ki-67+ malignant cells by IHC, F1-score=0.77) than the previously reported PAM50 classification (F1-score=0.62). In particular, the recall score increased significantly (0.85 in current classification versus 0.5 in previously reported), which can be highly important when it is necessary not to miss patients with the aggressive subtype (FIG. 10D). Luminal BG50 subtypes (Luminal A and Luminal B) almost entirely consist of samples with HR+/HER2− IHC phenotype (95% and 93% respectively, FIG. 8A).

To evaluate the impact of belonging to a certain BG50 subtype on survivability independently from grade and stage, COX regression on Metabric cohort was performed; Basal subtype was used as reference. When BG50 subtyping was the only feature used, the log hazard ratios were −0.79 and 0 for Luminal A and Luminal B Subtypes respectively (pval<0.005 for Luminal A). Performing regression with BG50 and tumor stage as features, the log hazard ratios changed insignificantly and became −0.66 and 0.05 for Luminal A and Luminal Brespectively (pval<0.005 for Luminal A). However, when BG50 and BG Grade [doi.org/10.1158/1538-7445.AM2022-1227] were used as features, the ratio changed more, becoming −0.29 and 0.14 for Luminal A and LumB. The result indicates that BG50 predicts survival of Luminal subtypes independent of tumor stage. However, BG50 and BG Grade are partially correlated features in terms of survival prediction, especially for Luminal A subtype, which consists almost entirely of low grade samples (FIG. 12K).

General trend for luminal subtypes is high expression of hormone receptor genes ESR1 and PGR; luminal marker genes GATA3 and FOXA1; kinase receptor genes ERBB3 and IGF1R; high expression of cell migration gene AGR3 along with low expression of CDH3; low expression of basal marker gene SOX11 and cell cycle genes CCNE1, MYBL2, CCNB2, CDC20 and RRM2. Luminal subtypes are also characterized by relatively high expression of genes TFF1, CA12, XBP1, MDM2 and low expression of gene PHGDH (FIGS. 8E and 8F). In terms of signaling, estrogen pathway is more active in luminal subtypes while EGFR, Hypoxia, MAPK, NFkB, PI3K and TNFa pathways are less active than in other subtypes (FIG. 8D).

### Luminal A

Luminal A is the least aggressive subtype with the most favorable prognosis. Five-year survival of Luminal A samples was the best in all three datasets: 93% in Metabric, 88% in TCGA and 93% in SCANB (FIG. 5C). Among all the genetic events found, only mutations in genes PIK3CA (60%, pval<1e-26), CDH1(18%, pval<1e-8) and MAP3K1 (17%, pval<1e-6) had high statistical significance by right tailed Fisher's exact test comparing Luminal A vs all (FIG. 8C).

Of all subtypes, Luminal A has the lowest expression of cell cycle genes MKI67, CDK1, CCNB1, CCNE1, MYBL2, CCNB2, CDC20, RRM2 and E2F2; and mitosis genes KIF2C, PTTG1, TPX2, NDC80, AURKB—expressions of these genes is significantly lower in Luminal A than in LumB. Hormone receptor gene PGR is expressed the highest in Luminal A subtype. Expression of basal marker gene FOXC1, cell cycle gene MYC and differentiation marker gene KRT17 is the second highest in Luminal A subtype (after Basal). Luminal A subtype can also be characterized by the lowest expression of UBE2T, UBE2C, TOP2A, BIRC5 and TYMS genes (FIGS. 8E and 8F).

Compared to other subtypes, Luminal A has the most active TGFb and WNT signaling; the least active PI3K signaling (FIG. 8D).

All the above-mentioned genes and pathways were significantly different in terms of expression and signaling for Luminal A comparing to each of other subtypes (pval<0.001, ad-hoc Kruskal-Wallis H-test with post-hoc Dwass-Steel-Crichtlow-Fligner's test).

Deconvolution profile of Luminal A subtype contains increased number of fibroblasts (higher than in Basal, HER2-low and LumB, pval<0.001; higher than in HER2-high, pval 0.0024) and endothelium (higher than in any other subtype, pval<0.001); decreased number of macrophages (lower than in any other subtype, pval<0.001) (FIG. 13A).

### Luminal B

Luminal B has many common features with Luminal A (see section “Luminal in general” above); however Luminal Bis more proliferative and aggressive. Five-year survival of Luminal B Samples in all three datasets is worse than Luminal A, but better than samples from other subtypes: 80% in METABRIC, 83% in TCGA and 88% in SCANB (FIG. 5C).

To identify genetic events common for Luminal B, right tailed Fisher's exact test comparing Luminal Bvs all was applied. Luminal B subtype can be characterized by mutations in AKT1 (5%, pval<1e-6) and GATA3 (17%, pval<1e-19).

Luminal B has the highest expression of luminal marker genes FOXA1 and GATA3, hormone receptor gene ESR1. Expression of kinase receptor ERBB3 is the highest in Luminal Bamong subtypes, while expression of EGFR is the lowest. Luminal B can also be characterized by the lowest expression of basal marker FOXC1 and differentiation marker KRT17. In terms of cell migration genes, Luminal B subtype is the leader in expression of AGR2 and AGR3, while CDH3 and CLDN8 are the least expressed in LumB. Of other features, Luminal B subtype can be characterized by the highest expression of genes XBP1, TFF1, CA12 and MDM2 (FIGS. 8E and 8F).

Among all the BG50 subtypes, Luminal B has the most active estrogen signaling; the least active hypoxia, MAPK, NFkB, TNFa and trail signaling (FIG. 8D).

Deconvolution profile of Luminal B subtype contains a decreased number of Secreting B cells (FIG. 13A).

### Normal

The Normal subtype was originally defined by including normal breast tissue samples alongside tumor samples during the molecular profiling process [nature.com/articles/s41523-023-00589-0]. This implies that the presence of normal tissue played a role in the identification and definition of this subtype. After careful investigation of histological slides of samples which were annotated as Normal-likes in TCGA it was revealed that they have a high amount of adipose tissue (FIG. 10A) and DCIS component. Although the TCGA purity analysis depicted a relatively lower mean purity for the Normal-like subtype compared to others, the purity for all samples steadfastly remained above 20%, implying a myoepithelial-like characteristic rather than significant tumor-free contamination (FIG. 10B). The adipose gene signature further corroborated this argument, revealing that while the adipose signature level in Normal-like samples was significantly lower than in normal samples, it mirrored that of the Luminal A subtype. Moreover, evaluations of proliferation and keratin signatures across the same groups demonstrated significantly higher scores in the Normal-like subtype compared to normal tissue, aligning closely with the Luminal A subtype (FIG. 12J).

The survival rate comparison showcased near-identical rates between Normal-like and Luminal A subtypes (92% and 93% respectively) (FIG. 10C). These collective findings, coupled with previous research highlighting the Normal-like tumors' shared IHC status with Luminal A subtype and their characterization by a normal breast tissue profiling, compellingly advocate for the reclassification of the Normal-like subtype. Despite the lack of a unique biological characterization, the evidence suggests that the Normal-like subtype is not tumor-free as previously hypothesized, thus warranting its reclassification as part of the Luminal A subtype.

### Clinical Applicability of New Classifier

[FIGS. 8A-8F] Patients' OS was evaluated with regards to the three different datasets. It was observed that METABRIC/TCGA datasets reflect natural features of each breast cancer subtype while the SCAN-B cohort showed the impact of current treatment strategies on these subtypes with regards to the time points when these datasets were collected.

The METABRIC and TCGA OS prediction curves were quite similar. They both show that Luminal A patients have better survival among all subtypes while HER2-high and HER2-low subtypes have the worst survival. At the same time Luminal B patients seem to have worse survival than Basal patients. These results were surprising since they do not fit the current view on the prognostic value of the different breast cancer subtypes. However, the METABRIC cohort was collected between 1977-2005 and TCGA between 2006-2013. The original annotation of these datasets was based on the primary pathology reports, with obvious differences in terminology for the classification of histological tumor types over time with respect to the differences in routine clinical practice. Since adjuvant chemotherapy was established as a standard only in the middle of 90s, prophylactic use of tamoxifen was approved in 1998 and prophylactic trastuzumab in 2011, METABRIC/TCGA OS curves may reflect the natural course of the breast cancer irrespective of treatment strategies.

The SCAN-B cohort was the most recent dataset with samples collected from 2014 to the present. It was assumed that these patients received hormonal, HER2-targeted and chemotherapy as clinically indicated. Therefore, the prognostic value of the SCAN-B appeared to be the most clinically important since it fits current treatment standards. The OS prediction shows that Luminal A patients had the best survival, then comes Luminal B, HER2-high, and Basal. HER2-low which was initially separated from non-basal patients, fell into a curve which is closer to the Basal cohort than to the others. METABRIC/TCGA curves for HER2-low also show the worst survival. It was assumed that HER2-low subtype was a separate biological entity which may require a treatment approach distinct from the current standards; it was confirmed in three different datasets.

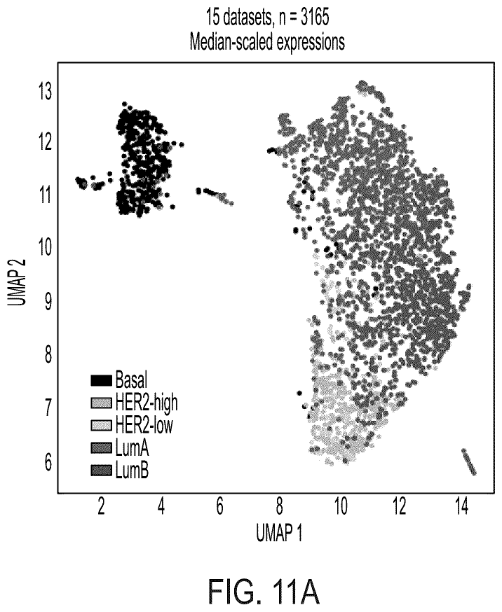

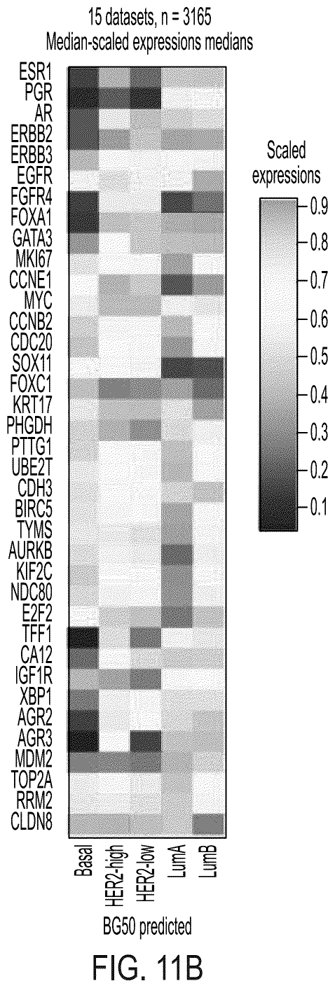

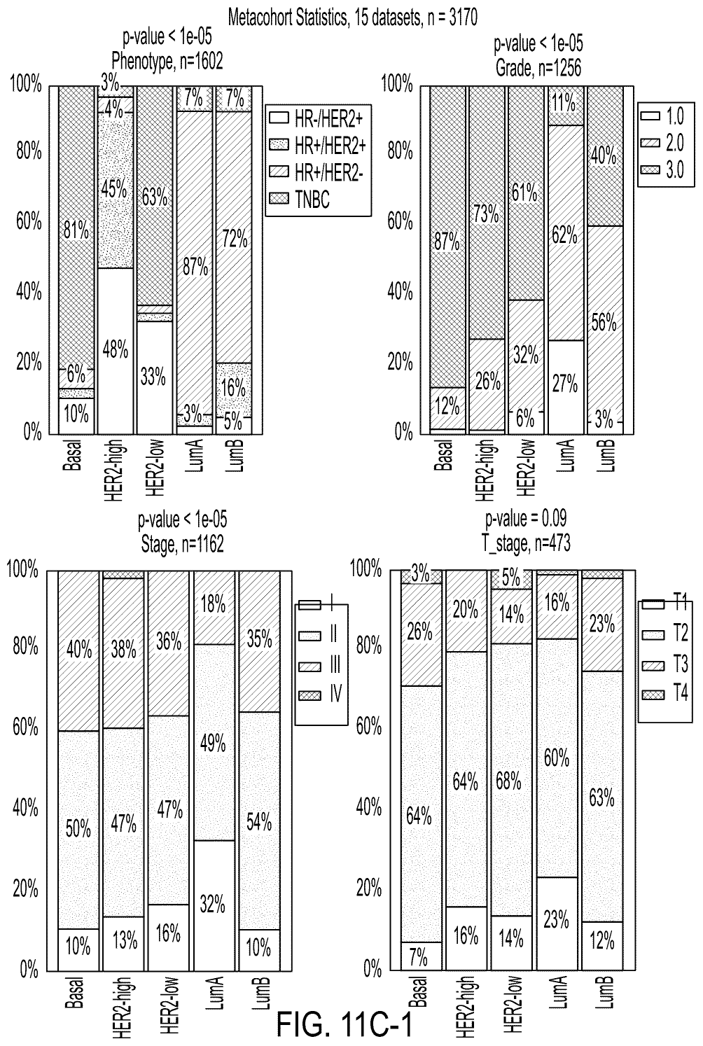

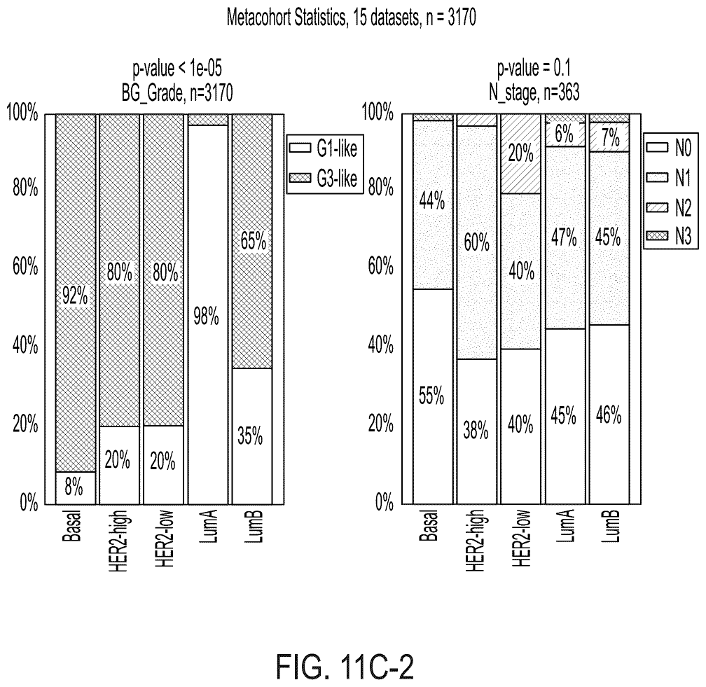

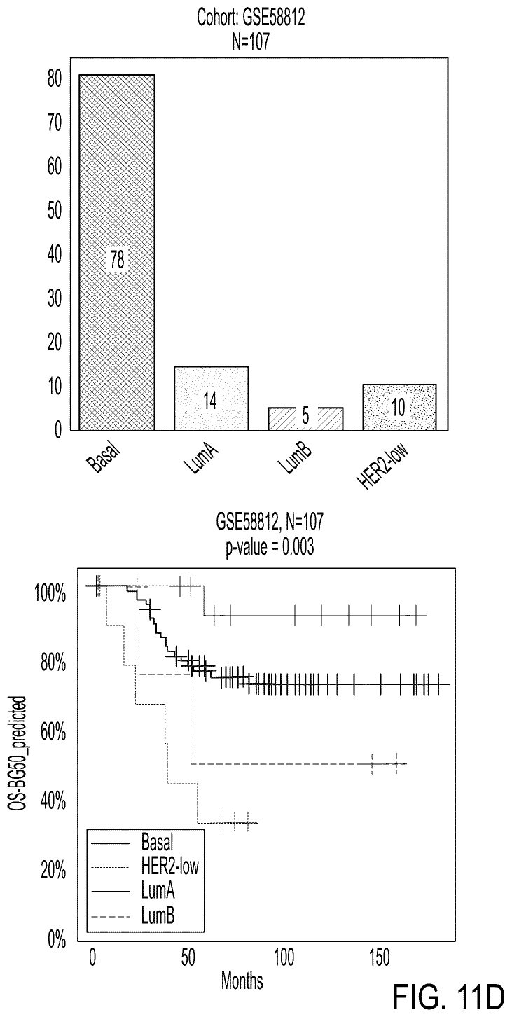

The hierarchical classifier was trained on TCGA and tested on SCANB datasets, which were manually annotated according to the invented BG50 classification. The classifier was subsequently applied to typing 3165 samples from 15 separate breast cancer datasets obtained on various microarray platforms. The UMAP of concatenated median-scaled gene expressions reveals clusters corresponding to predicted subtypes (FIG. 11A). Gene expression patterns of the predicted subtypes are similar to those of the initial classification (FIGS. 11B, 13E-1 and 13E-2). Survival prognosis was tested on TNBC dataset, 5-years survival for HER2-low is the worst result among other subtypes (FIG. 11D).

Then, the correlation of the obtained classification with the clinical annotation of the samples was studied. Most Luminal samples were HR+ by IHC (87% and 72% for Luminal A and Luminal Brespectively), HER2-high subtype consisted predominantly of HER2+ samples (93%), while Basal subtype was mostly TNBC (81%). In terms of proliferation, Luminal A subtype was almost entirely low-grade, while other subtypes were mostly high grade, especially Basal (92% G3-like). Tumor stage was roughly evenly distributed, with a slight skew towards low stages for Luminal A—32% of first stage samples (FIG. 11C-1 and FIG. 11C-2).

The BG50 classifier, applied to a comprehensive cohort of 6223 breast tumor samples, has not only provided a nuanced understanding of breast cancer subtypes but has also shone a spotlight on the distinct nature of the HER2-low subtype. Alongside this, the complexity surrounding the Normal subtype is addressed, adding depth to the discussion of breast cancer molecular subtyping.

The HER2-low subtype, as identified and characterized by the BG50 classifier, challenges the traditional binary classification of HER2 status in breast cancer. This subtype, often overshadowed by its HER2-high counterpart, emerges as a unique entity with distinct biological and clinical features. The HER2-low subtype demonstrates a molecular profile that, while sharing similarities with the HER2-high group, distinctly lacks the high expression of ERBB2 and adjacent GRB7 genes, distinguishing it from samples with HER2 amplification.

The HER2-low subtype's unique genetic landscape, particularly its high expression of EGFR and CLDN8 and its low expression of IGFR1, suggests alternative pathways driving its oncogenesis. This indicates potential avenues for targeted therapies beyond the conventional scope of HER2-targeted treatments. Moreover, the survival analysis within this subgroup, reflecting poorer outcomes compared to other subtypes, underscores the clinical significance of recognizing and appropriately treating this distinct subgroup.

Breast cancer, a heterogeneous disease with diverse molecular subtypes, demands individualized therapeutic strategies. The intrinsic molecular subtyping has transformed the understanding of the disease, leading to more targeted therapeutic approaches. In this context, the recently proposed BG50 classifier by BostonGene seems poised to provide deeper insights into this heterogeneity, particularly in its attempt to refine the categorization of breast cancer subtypes.

Historically, the PAM50 classifier has been the hallmark for intrinsic subtyping in breast cancer. Its clinical implications, however, have been limited to the prognostic assay in the early-stage HR+/HER2-negative disease and IHC-based concordance based on the recommendations emerging from the St. Gallen International Breast Cancer Conference in 2013. While IHC-based classification has practical utility, it may not fully capture the biological nuances of breast tumors, especially in metastatic disease. This was evident from several studies, where certain subtypes benefited inconsistently from therapy and had different survival rates among the same IHC phenotype.

Within HR-positive HER2-negative disease, Prat et al. in 2019 analyzed the intrinsic subtype using the PAM50 assay in patients treated in BOLERO-2, which was a phase III trial. The study found luminal A at 46.7% (n=122), HER2-enriched at 21.5% (n=56), luminal B at 15.7% (n=41), normal-like at 14.2% (n=37), and Basal at 1.9% (n=5). Luminal subtypes had a median PFS of 6.7 months, while non-luminal subtypes were at 5.2 months (adjusted hazard ratio of 0.66, 95% CI: 0.47-0.94, p=0.020). The HER2-enriched subtype exhibited a median PFS of 5.2 months, contrasted with 6.2 months for non-HER2-enriched subtypes (adjusted hazard ratio of 1.53, 95% CI: 1.07-2.19, p=0.019). When patients with HER2-enriched tumors were administered everolimus combined with exemestane, their median PFS improved significantly to 5.8 months, compared to 4.1 months when given placebo with exemestane (adjusted hazard ratio of 0.49, 95% CI: 0.26-0.90, p=0.034). However, the correlation between HER2-enriched tumors and the benefit of everolimus was found to be nonsignificant (p=0.433).

Other trials show similar distribution of intrinsic subtypes within HR-positive HER2-negative disease. When analyzing the PALOMA-3 trial, Turner et al. revealed that 44% of tumors were luminal A (n=133), 31% were luminal B (n=93), 1.66% were Basal (n=5), 20.86% were HER2-enriched (n=63), and 2.65% were normal-like (n=8). In the PALOMA-2 studyanalyzed by Finn et al., 50.3% of patients had luminal A tumors (n=229), 29.7% had luminal B (n=135), 18.7% were HER2E (n=85), 0.5% were Basal (n=2), and 0.9% were normal-like (n=4). The cumulative percentage of non-luminal subtypes in PALOMA-3 and PALOMA-2 was 25% and 20.1%, respectively.

Both studies showed that the addition of palbociclib heightened survival across all subtypes, especially benefitting luminal subtypes. In the PALOMA-3 study, luminal A patients experienced a notably prolonged median PFS of 16.6 months with palbociclib plus fulvestrant compared to 4.8 months with placebo, while luminal B patients observed a median PFS of 9.2 months versus 3.5 months, respectively. The PALOMA-2 trial echoed these findings, demonstrating that both luminal subtypes derived considerable benefit from adding palbociclib to letrozole. Contrastingly, patients with non-luminal hormone receptor-positive tumors in PALOMA-3 had a more modest median PFS improvement. Moreover, while the PALOMA-2 study showed the potential benefits of palbociclib in the HER2-like group, the results were constrained due to the small sample size. Collectively, these data underscore that luminal subtypes exhibit a more pronounced therapeutic benefit from endocrine therapy compared to their non-luminal counterparts.

The analysis of Prat et el. of MONALEESA trials, which encompassed MONALEESA-2, MONALEESA-3, and MONALEESA-7, executed a comprehensive PAM50-based analysis on tumor samples, elucidating the differential treatment responses based on tumor subtypes. Of the 1,160 tumors scrutinized, Luminal A was the predominant subtype with 542 patients (46.7%), trailed by Luminal B accounting for 278 patients (24.0%), Normal-like with 163 patients (14.0%), HER2-enriched (HER2E) represented by 147 patients (12.7%), and Basal with 30 patients (2.6%). This distribution was consistently observed across both the RIB and placebo arms and throughout each trial. It's imperative to highlight the robust association between tumor subtypes and progression-free survival (PFS) in both treatment categories (P<0.001). When contrasted to Luminal A, the disease progression risks surged notably for LumB, HER2E, and Basal subtypes by 1.44, 2.31, and 3.96 times, respectively. Regarding the therapeutic efficacy of RIB, every subtype except Basal showcased substantial PFS enhancement. In particular, the patients with HER2-enriched (HR, 0.39), Luminal B (HR, 0.52), Luminal A (HR, 0.63), and Normal-like (HR, 0.47) subtypes reaped the benefits of RIB. Conversely, the Basal group, constituting a minor cohort of 30 patients, failed to observe any therapeutic gains from RIB (HR, 1.15).

Another study that highlights the potential significance of intrinsic subtypes in clinical practice, was a retrospective analysis of the EGF30008 phase 3 clinical trial. Using the PAM50 classifier, 821 HR-positive HER2-negative samples were categorized based on their intrinsic subtypes. Although the progression-free survival (PFS) and overall survival (OS) findings were in line with existing data, a noteworthy discovery was the potential advantage for patients having a HER2-enriched profile, even if they were HER2-negative. These patients showed a pronounced benefit from the combination of lapatinib and endocrine therapy, with a median PFS 6.49 vs 2.60 months; progression-free survival hazard ratio, 0.238 [95% CI, 0.066-0.863]; interaction P=0.02). This suggests that patients with HR-positive HER2-negative breast cancer and a HER2-enriched profile might particularly benefit from combining lapatinib with endocrine therapy.

However, further insights are eagerly anticipated from the HARMONIA SOLTI-2101 trial where HR-positive HER2-negative patients with HER2-enriched or Basal breast cancer allocated either to the hormone therapy or paclitaxel and SOLTI-1303 PATRICIA where patients with HER2-positive disease and Luminal subtype receive palbociclib+trastuzumab. Nonetheless, these data support the idea that surrogate IHC phenotype has limited clinical significance at least in metastatic patients.

The analyzed studies confirm that the intrinsic subtype in HR+/HER2− mBC is a promising prognostic and predictive marker for therapeutic responsiveness. Their findings are in alignment concerning intrinsic subtype distributions, offering a comprehensive understanding of the biology behind HR-positive, HER2-negative conditions. While the consistent benefit derived by luminal subtypes from endocrine therapy aligns with expectations, the results pertaining to the HER2-enriched subtype warrant special attention. The elevated occurrence of HER2-enriched subtypes, paired with their evident advantage from endocrine therapy, may stem from the constraints of the renowned PAM50 classifier. Given that the PAM50 classifies tumors with highest activation of the EGFR-HER2 pathway into the HER2-enriched category without mandating HER2 amplification or overexpression, the proportion of HER2-enriched samples within these patients would possibly be fewer with more accurate allocation of non-HER2-amplified samples.

HER2-Enriched Vs. HER2-High Subtypes

The PAMELA study further investigated the potential of the HER2-enriched subtype as a biomarker for the treatment of HER2-positive early-stage breast cancer patients. Using the iconic PAM50 classification, the study aimed to determine whether patients with the HER2-enriched subtype would derive the most benefit from dual lapatinib+trastuzumab HER2 blockade. At the time of surgery, 41 (41%) of 101 HER2-enriched patients achieved a pathological complete response, compared to only 5 (10%) of the remaining 50 patients with non-HER2-enriched subtypes.

Early results from the study, conducted in 2017, indicated a promising role for the HER2-enriched subtype in identifying HER2-positive breast cancer patients likely to benefit from dual HER2 blockade therapies. However, a twist came in 2020 when the same group of scientists revealed further insights. They noted that while the HER2-enriched subtype showed some predictive value, its combination with high ERBB2 RNA expression was even more indicative of a pathological complete response.

They performed in-depth analysis across multiple clinical trials and revealed that the original HER2-enriched classification might be lacking. Of the 422 HER2-positive tumors examined from five clinical trials, HER2-E accounted for 83.8% of ERBB2-high tumors and 44.7% of ERBB2-low tumors. When patients were treated with lapatinib and trastuzumab, those in the HER2-E/ERBB2-high group had a significantly higher pCR rate of 44.5% compared to just 11.6% in other groups.

However, KEYRICHED-1 added another layer to this understanding. The study was focusing on the dual trastuzumab+pertuzumab anti-HER2 blockade in combination with the checkpoint inhibitor pembrolizumab for early-stage HER2-positive HER2-enriched patients. Centrally confirmed pCR-rate in HR+/HER2+ tumors was 38.5% compared to 58.5% in HR−/HER2+ tumors. Out of 48 patients selected to the trial, none of the 4 patients with IHC HER2 2+/ISH-positive status achieved a pCR, despite being HER2-enriched. In stark contrast, 20/39 (51.2%) of IHC HER2 3+ tumors achieved pCR. Centrally confirmed pCR-rate in HR+/HER2+ tumors was 38.5% compared to 58.5% in HR−/HER2+ tumors, indicating that the HER2-enriched subtype is a potent biomarker only in conjunction with HER2 IHC high expression

The evolving landscape of HER2-positive breast cancer treatment indicates a movement towards more tailored therapies, optimizing outcomes while minimizing toxicity. The evolving insights from the PAMELA study, coupled with those from KEYRICHED-1, underscore that while the HER2-enriched subtype derived from the original PAM50 classifier is a promising lead, it should be assessed in tandem with HER2 expression either measured via RNAseq or IHC as it envelops non-HER2-overexpressed/amplified tumors within the HER2-enriched category. This research evolution underscores the potential limitation of the original HER2-enriched subtype as a stand-alone biomarker. The BG50 classifier, which categorizes tumors into a HER2-high group based on high HER2 IHC expression or FISH amplification and HER2-low EGFR-driven group, could serve as a more effective tool.

### HER2-Low as a Distinct Biological Phenotype

The question of whether the HER2-low subtype stands as its own distinct clinical and biological category has been a focal point in contemporary oncological discussions. Validating the HER2-low subtype as a distinct entity hinges on addressing several critical considerations. These include the necessity for a tailored treatment approach specifically beneficial for this subtype, its distinct prognostic implications in clinical settings, and the presence of a unique biological signature supported by a reliable and robust assay for its identification.

The clinical relevance of HER2-low status took a significant leap with the unveiling of the phase III DESTINY-Breast04 trial's results aimed to contrast trastuzumab deruxtecan with chemotherapy in patients with HER2-low metastatic breast cancer who had received one or two previous lines of chemotherapy. Of the 557 randomized participants, 494 (88.7%) had hormone receptor-positive disease, while 63 (11.3%) were triple-negative. For the hormone receptor-positive cohort, median progression-free survival was 10.1 months versus 5.4 months, and overall survival stood at 23.9 months as opposed to 17.5 months. Evaluating the entire study cohort, median progression-free survival was 9.9 months in the trastuzumab deruxtecan group compared to 5.1 months, while overall survival rates were 23.4 months versus 16.8 months. Until now this is the first study that accentuated the clinical significance of identifying HER2-low status as the prior studies failed.

However, a shadow of doubt looms over these findings, as patients in the trial were allowed to provide their archival tumors for HER2 evaluation. A study by Y. Bar et al. further illuminated this notion, suggesting that HER2-low could be perceived more as a spectrum than a definitive biological entity. This research revealed that TNBC patients previously not identified as HER2-low, with subsequent biopsies upon disease progression heightened the probability of a HER2-low classification. This raises valid concerns regarding the real-time expression of HER2, given the documented dynamic and heterogeneous nature of HER2 status when assessed through IHC.

While many studies agree on the categorization of HER2-low BC as tumors with IHC 1+ or 2+ without gene amplification, its prognostic implications vary. While some studies indicate an impact of HER2-low on outcomes, others suggest no significant difference or variability based on other factors such as HR status or genomic risk either in early or metastatic disease. Multiple studies, including Tarantino et al, emphasize the importance of considering HR status within the HER2-low category because the proportion of HER2-low breast tumors was found to be higher in HR-positive disease either in early or metastatic breast cancer. Francesco Schettini et al. performed PAM50 analysis and observed a higher expression of ERBB2 and luminal-related genes in HER2-low tumors when compared to HER2 0 within HR-positive disease. No gene was found differentially expressed in TNBC according to HER2 expression, underlining the importance of hormonal receptor status in understanding this subtype.

The discrepancies between the studies may be addressed firstly to the question whether IHC or PAM50 is accurate in identification of the HER2-low subtype. A significant concern lies in the ever-evolving criteria for defining HER2-low status and the absence of central HER2 testing in the described studies. Moreover, the IHC technique's reliability has been undermined by its low diagnostic agreement—a mere 26% consistency between HER2 0 and HER2 1+ scores among expert pathologists. This is further accentuated when considering IHC's inability to address intra-tumor HER2 heterogeneity, which represents up to 40% of all breast tumors and has clinical and prognostic implications, with poor response to anti-HER2-based regimens and worse prognosis, compared to HER2-positive tumors.

The BG50 classifier has distinguished the HER2-low subtype as an entity with distinct clinical and biological features. Of note, this subtype is characterized by low HER2 IHC expression, suggesting trastuzumab deruxtecan as a potential therapeutic option for these tumors. When examining survival rates, the HER2-low subtype as classified by BG50 consistently exhibits the poorest outcomes across several datasets, including METABRIC, TCGA, and SCAN-B. This observation becomes even more compelling when the remarkable similarity between BG50's HER2-low and Burstein's LAR subtype (FIG. 13D), the latter being akin to the HER2-enriched intrinsic subtype within iconic PAM50 was recognized. Supporting this linkage, Agostinetto et al. reported a higher prevalence of HER2-enriched tumors within the HER2-low/TNBC cohort relative to the HER2-low/HR-positive group. Further underscoring its clinical significance, the LAR subtype of TNBC which congruent with BG50's HER2-low, is often associated with a dire prognosis, frequently presenting with lymphatic invasion and distant metastases, notably to bones.

To conclude, the BG50 classifier has highlighted the HER2-low subtype as a distinct category in breast cancer research. This new categorization offers a more nuanced approach to understanding and treating the disease.

Parallel to the findings on HER2-low, the analysis reveals complexities in the Normal subtype. This subtype's classification, potentially influenced by the presence of high normal cell content, calls for caution in interpreting molecular profiles. The presence of normal tissue, or a preinvasive component like DCIS, can skew the classification toward Normal-like or Luminal A subtypes. This highlights the importance of considering histological context and tumor purity in molecular subtyping.

Integrating these insights into the broader landscape of breast cancer classification underscores the evolving nature of molecular subtyping. The BG50 classifier, through its refined approach, not only aligns closely with the traditional PAM50 classification but also brings to light the nuances within subtypes like HER2-low. Future research should focus on validating these findings across diverse cohorts and exploring the therapeutic implications of these molecular subtypes.

In conclusion, the BG50 classifier enhances the understanding of breast cancer's molecular complexity. It brings forward the distinct nature of the HER2-low subtype, emphasizing its clinical importance and potential for targeted therapeutic approaches. Simultaneously, it acknowledges the challenges in interpreting the Normal subtype, reflecting the intricacy of breast cancer's molecular landscape. While IHC-based classification has practical utility, certain subtypes of breast cancer benefit inconsistently from therapy and have different survival rates among the same IHC phenotype suggesting that it has limited clinical significance at least in metastatic patients. The BG50 classifier offers a more refined categorization of breast tumors with the focus on Luminal A, Luminal B, Basal, HER2-high, and HER2-low subtypes with unique insights in therapeutic approaches and prognosis. Specifically, the BG50 classifier suggests that the HER2-low has distinct characteristics, showing poorer survival outcomes and potential benefit from treatments like trastuzumab deruxtecan. As science advances, the goal remains to tailor breast cancer treatment to the unique molecular characteristics of each subtype, optimizing patient outcomes in the era of precision oncology.

Table 9 lists the National Center for Biotechnology Information (NCBI) identifiers for genes described herein.

TABLE 9
NCBI Gene Table.

| Gene | NCBI Gene ID |
|---|---|
| FOXA1 | 3169 |
| MLPH | 79083 |
| FOXC1 | 2296 |
| SFRP1 | 6422 |
| NAT1 | 9 |
| ORC6 | 23594 |
| BIRC5 | 332 |
| CDC20 | 991 |
| AGR2 | 10551 |
| AR | 367 |
| CA12 | 771 |
| CDK1 | 983 |
| ESR1 | 2099 |
| MYC | 4609 |
| PHGDH | 26227 |
| ACTR3B | 57180 |
| CDH3 | 1001 |
| KRT14 | 3861 |
| KRT5 | 3852 |
| EGFR | 1956 |
| CDC6 | 990 |
| FGFR4 | 2264 |
| MAPT | 4137 |
| CXXC5 | 51523 |
| MDM2 | 4193 |
| CCNE1 | 898 |
| MYBL2 | 4605 |
| RRM2 | 6241 |
| NUF2 | 83540 |
| TYMS | 7298 |
| ANLN | 54443 |
| UBE2T | 29089 |
| CENPF | 1063 |
| PTTG1 | 9232 |
| UBE2C | 11065 |
| MELK | 9833 |
| GRB7 | 2886 |
| ERBB2 | 2064 |
| FABP7 | 2173 |
| GATA3 | 2625 |
| CCNB2 | 9133 |
| CLDN8 | 9073 |
| ERBB3 | 2065 |
| IGF1R | 3480 |
| PIK3CA | 5290 |
| TOP2A | 7153 |
| KRT17 | 3872 |
| BCL2 | 596 |
| PGR | 5241 |
| SLC39A6 | 25800 |
| BLVRA | 644 |
| EXO1 | 9156 |
| MKI67 | 4288 |
| KIF2C | 11004 |
| CEP55 | 55165 |
| ACE2 | 59272 |
| AKR1B15 | 441282 |
| AGR3 | 155465 |
| E2F2 | 1870 |
| SOX11 | 6664 |
| TFF1 | 7031 |
| TWIST1 | 7291 |
| SOX10 | 6663 |
| GPR160 | 26996 |
| TFAP2B | 7021 |
| ELF5 | 2001 |
| LBP | 3929 |
| BAG1 | 573 |
| PPARG | 5468 |
| WNK4 | 65266 |
| VIM | 7431 |
| MMP11 | 4320 |
| CTNNA1 | 1495 |
| KIT | 3815 |
| CAV1 | 857 |
| FABP4 | 2167 |
| CCNB1 | 891 |
| AURKB | 9212 |
| AURKA | 6790 |
| PLK1 | 5347 |
| MCM2 | 4171 |
| BUB1 | 699 |
| E2F1 | 1869 |
| GNG12 | 55970 |
| SOCS5 | 9655 |
| CRY2 | 1408 |
| ELN | 2006 |
| PTPN21 | 11099 |
| COL14A1 | 7373 |
| ZNF608 | 57507 |
| ZCCHC24 | 219654 |
| AASS | 10157 |
| NDC80 | 10403 |

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as an example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as an example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

1. A method for identifying HER2-low breast cancer from RNA expression data of a tumor sample from a subject having breast cancer using a plurality of trained machine learning classifiers including first, second, and third trained machine learning classifiers associated with respective first, second, and third sets of genes, the method comprising:
using at least one computer hardware processor to perform:
obtaining the RNA expression data, the RNA expression data specifying RNA expression levels at least for genes in the first, second, and third sets of genes, the RNA expression data having been previously obtained from the tumor sample;
determining, using the RNA expression levels for the first set of genes and the first trained machine learning classifier, whether the tumor sample has a Basal molecular subtype;
when it is determined that the tumor sample does not have the Basal molecular subtype, determining, using the RNA expression levels for the second set of genes and the second trained machine learning classifier, whether the tumor sample has a HER2-high molecular subtype; and
when it is determined that the tumor sample does not have the HER2-high molecular subtype, determining, using the RNA expression levels for the third set of genes and the third trained machine learning classifier, that the tumor sample has a HER2-low molecular subtype.

2. The method of claim 1, further comprising:
when it is determined that the tumor sample has the HER2-low molecular subtype, recommending that the subject be treated with trastuzumab deruxtecan and/or administering the trastuzumab deruxtecan to the subject.

3. (canceled)

4. The method of claim 1,
wherein the first set of genes includes at least some genes selected from the group consisting of FOXA1, MLPH, FOXC1, SFRP1, NAT1, ORC6, BIRC5, CDC20, AGR2, AR, CA12, and CDK1, and
wherein determining whether the tumor sample has a Basal molecular subtype comprises:
generating a first input using expression levels for genes in the first set of genes, and
processing the first input using the first trained machine learning classifier to obtain a first output indicative of whether the tumor sample has the Basal molecular subtype.

5. (canceled)

6. The method of claim 1, wherein at least one of the first trained machine learning classifier, the second trained machine learning classifier, and the third trained machine learning classifier is a gradient boosted decision tree classifier.

7. (canceled)

8. The method of claim 1,
wherein the second set of genes includes at least some genes selected from the group consisting of MLPH, ESR1, FOXC1, MYC, PHGDH, ACTR3B, CDH3, KRT14, KRT5, EGFR, CDC6, FGFR4, MAPT, CXXC5, NAT1, MDM2, CCNE1, MYBL2, RRM2, NUF2, TYMS, ANLN, UBE2T, CENPF, PTTG1, UBE2C, CDC20, MELK, GRB7, ERBB2, FABP7, GATA3, AR, CCNB2, CDK1, CLDN8, ERBB3, IGF1R, PIK3CA, and TOP2A, and
wherein determining whether the tumor sample has a HER2-high molecular subtype comprises:
generating a second input using expression levels for genes in the second set of genes, and
processing the second input using the second trained machine learning classifier to obtain a second output indicative of whether the tumor sample has the HER2-high molecular subtype.

9-11. (canceled)

12. The method of claim 1,
wherein the third set of genes includes at least some genes selected from the group consisting of FOXA1, MLPH, ESR1, MYC, PHGDH, ACTR3B, SFRP1, KRT17, KRT5, EGFR, CDC6, FGFR4, MAPT, CXXC5, BCL2, PGR, NAT1, SLC39A6, BLVRA, CCNE1, MYBL2, RRM2, ORC6, NUF2, TYMS, ANLN, CENPF, EXO1, MKI67, BIRC5, UBE2C, KIF2C, CEP55, MELK, ERBB2, ACE2, FABP7, AKR1B15, GATA3, AGR3, AR, CA12, CDK1, CLDN8, E2F2, IGF1R, SOX11, TFF1, and TOP2A, and
wherein determining whether the tumor sample has a HER2-low molecular subtype comprises:
generating a third input using expression levels for genes in the third set of genes, and
processing the third input using the third trained machine learning classifier to obtain a third output indicating that the tumor sample has the HER2-low molecular subtype.

13-18. (canceled)

19. A system, comprising:
at least one computer hardware processor; and
at least one non-transitory computer-readable storage medium storing processor-executable instructions that, when executed by the at least one computer hardware processor, cause the at least one computer hardware processor to perform the method of claim 1.

20. At least one non-transitory computer-readable storage medium storing processor-executable instructions that, when executed by at least one computer hardware processor, cause the at least one computer hardware processor to perform the method of claim 1.

21. A method for identifying a molecular subtype of breast cancer for a subject, the method comprising:
using at least one computer hardware processor to perform:
obtaining RNA expression data, the RNA expression data having been previously obtained from a tumor sample from a subject having breast cancer; and
identifying, from among multiple breast cancer molecular subtypes and using the RNA expression data and a plurality trained machine learning classifiers, a molecular subtype for the tumor sample, the multiple breast cancer molecular subtypes comprising: a Basal subtype, a HER2-high subtype, a HER2-low subtype, Luminal subtype A, and Luminal subtype B.

22. The method of claim 21, further comprising:
identifying a cancer therapy for the subject based on the identified molecular subtype for the tumor sample from the subject and/or administering the cancer therapy to the subject.

23-25. (canceled)

26. The method of claim 21,
wherein the plurality of trained machine learning classifiers includes a first trained machine learning classifier associated with a first set of genes,
wherein the RNA expression data specifies RNA expression levels for genes in the first set of genes, and
wherein identifying the molecular subtype for the tumor sample comprises:
determining, using the RNA expression levels for the first set of genes and the first trained machine learning classifier, whether the tumor sample has the Basal molecular subtype.

27. The method of claim 26, wherein the first set of genes includes at least some, optionally all, of the following genes: FOXA1, MLPH, FOXC1, SFRP1, NAT1, ORC6, BIRC5, CDC20, AGR2, AR, CA12, and CDK1.

28. (canceled)

29. The method of claim 26,
wherein the plurality of trained machine learning classifiers includes a second trained machine learning classifier associated with a second set of genes,
wherein the RNA expression data specifies RNA expression levels for genes in the second set of genes, and
wherein identifying the molecular subtype for the tumor sample comprises:
when it is determined that the tumor sample does not have the Basal molecular subtype, determining, using the RNA expression levels for the second set of set of genes and the second trained machine learning classifier, whether the tumor sample has the HER2-high molecular subtype.

30. The method of claim 29, wherein the second set of genes includes at least some, optionally all, of the following genes: MLPH, ESR1, FOXC1, MYC, PHGDH, ACTR3B, CDH3, KRT14, KRT5, EGFR, CDC6, FGFR4, MAPT, CXXC5, NAT1, MDM2, CCNE1, MYBL2, RRM2, NUF2, TYMS, ANLN, UBE2T, CENPF, PTTG1, UBE2C, CDC20, MELK, GRB7, ERBB2, FABP7, GATA3, AR, CCNB2, CDK1, CLDN8, ERBB3, IGF1R, PIK3CA, and TOP2A.

31. (canceled)

32. The method of claim 26,
wherein the plurality of trained machine learning classifiers includes a third trained machine learning classifier associated with a third set of genes,
wherein the RNA expression data specifies RNA expression levels for genes in the third set of genes, and
wherein identifying the molecular subtype for the tumor sample comprises:
when it is determined that the tumor sample does not have the HER2-high molecular subtype, determining, using the RNA expression levels for the third set of set of genes and the third trained machine learning classifier, whether the tumor sample has the HER2-low molecular subtype.

33. The method of claim 32, wherein the third set of genes includes at least some, optionally all, of the following genes: FOXA1, MLPH, ESR1, MYC, PHGDH, ACTR3B, SFRP1, KRT17, KRT5, EGFR, CDC6, FGFR4, MAPT, CXXC5, BCL2, PGR, NAT1, SLC39A6, BLVRA, CCNE1, MYBL2, RRM2, ORC6, NUF2, TYMS, ANLN, CENPF, EXO1, MKI67, BIRC5, UBE2C, KIF2C, CEP55, MELK, ERBB2, ACE2, FABP7, AKR1B15, GATA3, AGR3, AR, CA12, CDK1, CLDN8, E2F2, IGF1R, SOX11, TFF1, and TOP2A.

34. (canceled)

35. The method of claim 26,
wherein the RNA expression data specifies RNA expression levels for genes in fourth and fifth sets of genes, and
wherein identifying the molecular subtype for the tumor sample comprises:
when it is determined that the tumor sample does not have the HER2-low molecular subtype, identifying that the tumor sample has a Luminal A or Luminal B subtype at least in part by:
determining a first enrichment score for the fourth set of genes;
determining a second enrichment score for the fifth set of genes;
providing the first and second enrichment scores as inputs to a logistic regression model to obtain an output indicating whether the tumor sample is Ki67 positive or Ki67 negative;
when it is determined, based on the output of the logistic regression model, that the tumor sample is Ki67 negative, determining that the tumor sample has the Luminal A molecular subtype; and
when it is determined, based on the output of the logistic regression model, that the tumor sample is Ki67 positive, determining that the tumor sample has the Luminal B molecular subtype.

36. The method of claim 35,
wherein the fourth set of genes consists of at least some, optionally all, of the following genes: CCNB1, AURKB, AURKA, PLK1, MCM2, BUB1, E2F1, MKI67, MYBL2, and
wherein the fifth set of genes consists of at least some, optionally all, of the following genes: GNG12, SOCS5, CRY2, ELN, PTPN21, COL14A1, ZNF608, ZCCHC24, AASS.

37-40. (canceled)

41. A system, comprising:
at least one computer hardware processor; and
at least one non-transitory computer-readable storage medium storing processor-executable instructions that, when executed by the at least one computer hardware processor, cause the at least one computer hardware processor to perform the method of claim 21.

42. At least one non-transitory computer-readable storage medium storing processor-executable instructions that, when executed by at least one computer hardware processor, cause the at least one computer hardware processor to perform the method of claim 1.
